(19) United States
(12) Patent Application Publication
(10) Pub. No.: US 2014/0163118 A1
(43) Pub. Date: Jun. 12, 2014

(54) Expression Signatures of Genes and Gene Networks Associated with Skin Aging

(71) Applicants: Giammaria Giuliani, Milan (IT); Raymond Rodriguez, Davis, CA (US); Somen Nandi, Davis, CA (US)

(72) Inventors: Giammaria Giuliani, Milan (IT); Raymond Rodriguez, Davis, CA (US); Somen Nandi, Davis, CA (US)

(73) Assignee: DERMACHIP INC., Davis, CA (US)

(21) Appl. No.: 14/115,352

(22) PCT Filed: May 3, 2012
(86) PCT No.: PCT/US12/36226

§ 371 Date: Jan. 27, 2014

Related U.S. Application Data
(60) Provisional application No. 61/482,071, filed on May 3, 2011.

(51) Int. Cl. G01N 33/50

(52) U.S. Cl. CPC G01N 33/5023
USPC 514/789; 435/6.13; 435/6.11

(57) ABSTRACT

Described herein are expression signatures of genes and coordinately expressed gene networks associated with skin aging, methods used to determine the physiological age of skin, and methods used to screen for compounds used to reduce the visible signs of aging of the skin.

### RELATION TO OTHER APPLICATIONS

This application claims the benefit of and priority to U.S. Provisional application No. 61/482,071 filed 3 May 2011.

### GOVERNMENT SPONSORSHIP

None

### FIELD OF THE INVENTION

The invention relates to newly discovered expression signatures of coordinately expressed genes and gene networks associated with the physiological and pathological processes of skin aging and to methods for identifying and using compounds that regulate and in some cases recalibrate gene sets and gene pathways associated with skin ageing wherein such regulation and recalibration effectively reverses the effects of ageing of the skin to produce more youthful looking skin so that the skin is more supple, smooth and hydrated and has less wrinkles and fine lines typically associated with ageing.

### BACKGROUND

Skin aging is natural process associated with a number of pathophysiologies that can reduce quality of life and longevity. The goal of this study is to gain new insights into how skin ages as a function of time. The working hypothesis for this study is that skin aging results from the dysregulation of key regulatory genes (i.e., gene signatures) that control thousands of genes in multiple, interacting and overlapping networks and pathways. Dysregulation of gene functions leads to physiological aging of tissues, cells and subcellular components. This dysregulation may be the result of damage to the products of key regulatory genes (i.e., oxidative damage to regulatory proteins) or to transcriptional silencing of these genes by mutagenic and/or epigenetic processes. This study is the first of its kind to examine, in a comprehensive fashion, the gene expression profiles in human skin (specifically adult females who self-identified themselves as white) of various age and to identify gene networks and gene signatures associated with the aging process. This information should yield a small number of robust and predictive genes/gene-network whose expression can be used to: (a) understand the relationship between chronological age and physiological age and (b) assess the ability of compounds (e.g., drugs, natural products and dietary factors) to regulate and/or dysregulate genes and gene networks. The present study provides information that will guide future research and suggest new therapies for preventing, mitigating or treating skin pathologies related to aging.

The skin is the largest human organ comprising about one sixth of total body weight. The skin performs a complex role in human physiology: serves as a barrier to the environment, and the sebum produced by some of its glands (sebaceous) have anti-infective properties. The skin acts as a channel for communication to the outside world, protects us from water loss, friction wounds, and impact wounds and uses specialized pigment cells to protect us from ultraviolet rays of the sun. Skin produces vitamin D in the epidermal layer, when it is exposed to the sun's rays. The skin helps regulate body temperature through sweat glands and helps regulate metabolism. The skin consists of three functional layers: Epidermis, the Dermis (or corium) and the Subcutis (or hypodermis).

Various cell types are present in the skin. Keratinocytes is the most abundant cell type in the epidermis. These cells produce keratin proteins. Fibroblasts differentiate into cells that form the dermis and produce collagen and elastin. Melanocytes produce the pigment melanin that accumulates around the nuclei of the keratinocytes absorbing harmful ultraviolet (UV) light. Langerhans cells (macrophages) reside in the dermis mediating humoral and cellular immune functions. Merkel's cells, which are present in small numbers but are more numerous in the skin of the palms and soles of the feet, are sensory mechanical receptors that respond to certain stimuli such as pressure or touch. The epidermis is the outermost skin layer. As skin cells migrate to the surface, farther away from their source of nourishment, they flatten and shrink. They lose their nuclei, move out of the basal layer to the horny layer (stratum corneum), and die. This process, called keratinization, takes about 4 weeks. About 10 percent of epidermal cells are melanocytes that pigment the skin. The epidermis is differentiated into five layers: horny layer (stratum corneum); clear layer (stratum lucidum); granular layer (stratum granulosum); prickle-cell layer (stratum spinosum); and the basal layer (stratum basale). The dermis is the layer just below the outer keratinized epidermal layer. The dermis contains cells, water, collagen fibers, glycosaminoglycans and fibronectins that form a hydrated gel and are responsible for the high elasticity and tensile strength of the dermis. Embedded in this layer are lymph channels, blood vessels, nerve fibers, muscle cells, hair follicles, sebaceous glands, and sweat glands.

Glycosaminoglycans are mucopolysaccharides present in the dermis that can bind large amounts of water. As the skin ages, the interweaving of the collagen fibers increases and the water-binding capacity diminishes and the skin tends to wrinkle. Glycosaminoglycans bind with the proteins in the connective tissue matrix to form proteoglycans. These proteoglycans form a gel-like material that can absorb and expel water like a sponge. Glycosaminoglycans are subject to a continuous turnover. In contrast, the collagen fibers are only renewed when necessary, such as when injury is sustained. The ability of the skin to store water and thereby remain soft and supple depends in part on the presence of lipids, arginine, and other “natural moisturizing factors” (NMF) that originate from the cornification (differentiation) of the keratinocytes, for example, pyrrolidine carboxylic acid, and secretions from the sweat and sebaceous glands including urea, salts, and organic acids.

The dermis also contains collagens. Type I collagen is the most abundant protein in skin connective tissue, which also contains other types of collagen (III, V, VII), elastin, proteoglycans, fibronectin, and other extracellular matrix proteins. Newly synthesized type I procollagen is secreted into the dermal extracellular space where it undergoes enzymatic-processing, arranging itself into a triple helix configuration. The triple helix complexes associate with other extracellular matrix proteins such as leucine-rich small proteoglycans, to form regularly arranged fibrillar structures. This process, called fibrillogenesis, results in formation of collagen bundles that are responsible for the strength and resiliency of the skin.

Skin aging is influenced by several factors, including genetics, environmental exposure (ultraviolet (UV) irradiation, xenobiotics, and mechanical stress), hormonal changes, and metabolic processes (generation of reactive chemical compounds such as activated oxygen species, sugars, and aldehydes). Taken together, these factors lead to cumulative alterations of skin structure, function, and appearance. The influence of the environment, especially solar UV irradiation, is of considerable importance for skin aging. Skin aging due to UV exposure (photoaging) is superimposed on chronological skin aging. Historically, scientists considered photoaging and chronological skin aging as two distinct entities. Although the typical appearance of photoaged and chronologically aged human skin can be readily distinguished, recent evidence indicates that chronologically aged and UV-irradiated skin share important molecular features including altered signal transduction pathways that promote matrix-metalloproteinase (MMP) expression, decreased procollagen synthesis, and connective tissue damage. This concordance of molecular mechanisms suggests that UV irradiation accelerates many key aspects of the chronological aging process in human skin. Based on this relationship between UV irradiation and chronological aging, acute UV irradiation of human skin may serve as a useful model to study molecular mechanism of skin chronological aging.

At the tissue level, chronologically aged skin shows general atrophy of the extracellular matrix reflected by decreased number of fibroblasts, and reduced levels of collagen and elastin. The organization of collagen fibrils and elastin fibers is also impaired. This impairment is thought to result from both decreased protein synthesis that particularly affects types I and III collagens in the dermis and increased breakdown of extracellular matrix proteins.

Photodamaged skin is associated with increased epidermal thickness and alterations of connective tissue organization. The hallmark of photoaged skin is accumulation of amorphous elastin-containing material that resides beneath the epidermal dermal junction. Impairment of the fibrillar organization of collagen and elastin is typically more severe in photoaged skin, compared to sun-protected chronologically aged skin. The severity of photoaging is proportional to accumulated sun exposure and inversely related to the degree of skin pigmentation. Individuals with fair skin are more susceptible to solar UV-induced skin damage than darker-skinned individuals.

At the cellular level, one of the earliest detectable responses of human skin cells to UV irradiation is activation of multiple cytokine and growth factor cell surface receptors, including epidermal growth factor receptor (EGF-R), tumor necrosis factor (TNF) alpha receptor, platelet activating factor (PAF) receptor, insulin receptor, interleukin (IL-1) receptor, and platelet-derived growth factor (PDGF) receptor.

Activation of cell surface cytokine and growth factor receptors results in recruitment in cytoplasm of adaptor proteins that mediate downstream signaling. Assembly of these signaling complexes results in activation of small GTP-binding protein family members which are key upstream regulators of the certain MAP kinases. The action of certain GTP-binding proteins results in an increased formation of superoxide anions. This increased production of ROS likely participates in amplification of the signal leading to the activation of the downstream enzyme complexes such as MAP kinase. ROS are necessary participants in multiple MAP kinase pathways.

Increased intracellular ceramide content may also contribute to activation of the MAP kinase pathways by UV irradiation. UV-induced ceramide generation seems to be dependent on increased ROS production, since ceramide and ROS levels rise in parallel, and UV-induced ceramide production is inhibited by the free radical scavenger Vitamin E. MAP kinase activation results in induction of transcription factor AP-1 that is a major effector of the MAP kinase pathways. AP-1 regulates expression of many genes involved in the regulation of cellular growth and differentiation. Transcription of several MMP (matrix-metalloproteinase) family members is strongly regulated by AP-1. Several MMPs are upregulated by AP-1. These include MMP-1 (interstitial collagenase or collagenase 1) which initiates degradation of types I and III fibrillar collagens, MMP-9 (gelatinase B), which further degrades collagen fragments generated by collagenases, and MMP-3 (stromelysin 1), which degrades type IV collagen of the basement membrane and activates pro-MMP-1. MMP induction is, in part, responsible for UV-induced damage to skin connective tissue. Together, MMP-1, MMP-3, and MMP-9 have the capacity to completely degrade mature fibrillar collagen in skin. Consistent with this, increased collagen breakdown has been demonstrated within 24 h after UV irradiation in human skin in vivo. Thus, UV irradiation of human skin causes extracellular matrix degradation via induction of transcription factor AP-1 and subsequent increased MMP production.

In addition to causing collagen breakdown, UV irradiation impairs new type I collagen synthesis. UV irradiation has been shown to decrease collagen production and impair organization of collagen fibrils in skin in vivo. In addition, increased breakdown of extracellular matrix proteins is also observed in UV-irradiated fibroblasts in vitro and in human skin in vivo. Down-regulation of type I collagen is mediated in part by UV-induced AP-1, which negatively regulates transcription of both genes that encode for type I procollagen (COL1A1 and COL1A2).

UV-induced down-regulation of collagen synthesis also occurs via paracrine mechanisms involving transforming growth factor-beta (TGF-beta) and other cytokines. TGF-beta is a major profibrotic cytokine, which regulates multiple cellular functions including differentiation, proliferation, and induction of synthesis of extracellular matrix proteins. The biological effects of TGF-beta are diverse and strongly dependent on its expression pattern and cell type. In human skin, TGF-beta inhibits growth of epidermal keratinocytes and stimulates growth of dermal fibroblasts. Moreover, TGF-beta induces synthesis and secretion of the major extracellular matrix proteins collagen and elastin. TGF-beta also inhibits expression of certain specific enzymes involved in the breakdown of collagen, including MMP-1 and MMP-3. TGF-beta also has the ability to affect gene expression by epigenetic modification of DNA. Exogenous TGF-beta was shown to induce and maintain expression of Foxp3 in regulatory T cells by demethylating a highly conserved region of the Foxp3 gene called Treg-specific demethylation region (TSDR) [J. K. Polansky et al., 2008. Eur. J. Immunol. 38: 1654-1663]. Both aging and UV irradiation induce molecular alterations that create skin aging. A major feature of aged skin is the reduction of types I and III procollagen synthesis. This reduction results in skin thinning and increased fragility. Both types I and III procollagen mRNA and protein expression are reduced in aged skin.

In addition to impaired collagen synthesis, increased production of several MMP family members, including MMP-1, MMP-2 (gelatinase A), MMP-3, and MMP-9 occurs in chronologically aged skin. With the exception of MMP-2, these MMPs are regulated by AP-1 and induced by UV irradiation. Interestingly. AP-1 expression is increased in aged human skin in vivo and aged skin fibroblasts in vitro. Oxidative stress is thought to be of primary importance in driving the aging process. The free radical theory of aging, first proposed several decades ago, envisions that the molecular basis of aging derives from accumulation, over a lifetime, of oxidative damage to cells resulting from excess ROS, which are produced as a consequence of aerobic metabolism. Although skin possesses extremely efficient anti-oxidant activities, it has been demonstrated that during aging, ROS levels rise and anti-oxidant defenses decline. ROS are necessary participants in multiple MAP kinase pathways. MAPK activation results in induction of AP-1, which in turn, upregulates expression of MMPs. This scenario provides a plausible mechanism for the observed increased collagen degradation in aged human skin.

In spite of existing differences, many critical molecular features of aged and UV-irradiated human skin bear striking similarities. It could be stated that these similarities reflect the central role that oxidative stress plays in UV irradiation-induced responses and aging in human skin. Viewed in this light, it is not surprising that UV irradiation and aging evoke similar molecular responses, since both are responding to oxidative stress. Nor is it surprising that the consequences of UV irradiation and aging have similar damaging impact on skin connective tissue.

### SHORT DESCRIPTION OF THE INVENTION

The invention relates to expression signatures of genes and to coordinately expressed gene networks associated with skin aging. Additionally the invention relates to methods used to determine the physiological age of skin, and methods used to screen for compounds used to reduce the visible signs of aging of the skin.

The inventors have used high throughput expression screening and SAGE (Serial Analysis of Gene Expression) analysis to compare sets of skin tissue from two cohorts of individuals of substantially different ages. The inventors have discovered a large number of previously unknown expression signatures that are related to age. Additionally they have discovered a large and complex network of coordinately expressed genes and pathways that are associated with the physiological and pathological process of skin aging. These expression signatures are used to (i) determine the physiological age of skin, (ii) screen for compounds and compositions that are used to reduce the visible signs of aging of the skin, particularly to the prevention and reduction of skin wrinkles and to the production and maintenance of youthful looking skin.

Additionally, using the methods of the invention, compounds may be identified by the methods of the invention which compounds affect the expression of various genes within the skin that are involved in chronological-induced and UV-induced skin damage. The invention relates to methods for recalibrating the expression of genes, genetic networks, and cellular pathways in the human skin, primarily in the dermis, that have changed as a result of the chronological aging process. The invention also relates to combinations of natural compounds that produce synergistic effects on the expression of genes relevant to the reversal of skin aging and skin cancer risk reduction.

### DESCRIPTION OF THE FIGURES AND TABLES

FIG. 1. A regulatory module responsible for a single gene signature in one aging-related network. White=up-regulated, grey=down-regulated). This network which created using only top 100 up & down regulated genes illustrates the complexity of the aging process. This network is connected to other networks involved in skin aging as well as to aging in other tissues. It is the gene signatures in different but connected and interacting networks that determine the aging process. It is the genetic regulatory module (e.g., IL-8, IFNG) that will serve as targets for drugs and natural compounds to reverse skin aging. It is believed that the loss of connections between networks reduces the ability of the cell to make coordinated responses to external stimuli and thus the cause of disease and aging.

FIG. 2. Master regulators correlate with the differential gene expression signatures between old and young cohorts. The X axis represents age from older (on the left) to younger (on the right). For each regulator (acronym boxed on the left hand side) two rows are shown, one of up-regulated genes and one of downregulated genes. It is very clear from this figure that certain genes and gene stets are upregulated in older skin samples and certain genes and gene sets are upregulated in younger skin samples. Low expression level in old cohorts is at the left of the bottom row which shows expression levels gradually increased to the right. The regulator gene symbols are listed at left side of the graph inside boxes. Each regulator has two rows of up and down regulated genes that are positioned according to their gene expression levels in the data sets. The P-value of the correlations between the gene expression signature of overall datasets (bottom row) and individual regulation patterns are listed right below each gene symbol. The lower the P-value, the high possibility the regulator has to be responsible for the signature changes. STAT1=a member of the Signal Transducers and Activators of Transcription family of transcription factors. CEBPD=a CCAAT/enhancer-binding protein delta is a protein that in humans is encoded by the CEBPD gene. The protein encoded by this intronless gene is a bZIP transcription factor which can bind as a homodimer to certain DNA regulatory regions. MAF=a transcription factor that in humans is encoded by the MAF gene. RXRA=Retinoid X receptor alpha (RXR-alpha), also known as NR2B1 (nuclear receptor subfamily 2, group B, member 1) is a nuclear receptor. NR1H3=Liver X receptor alpha (LXR-alpha) is a nuclear receptor protein. TFE3=Transcription factor E3. NFATC1=Nuclear factor of activated T-cells, cytoplasmic 1. RBL2=Retinoblastoma-like protein 2. SMAD3=Mothers against decapentaplegic homolog 3. NR2F1=Nuclear Receptor subfamily 2, group F, member 1 is member of nuclear hormone receptor family of steroid hormone receptors.

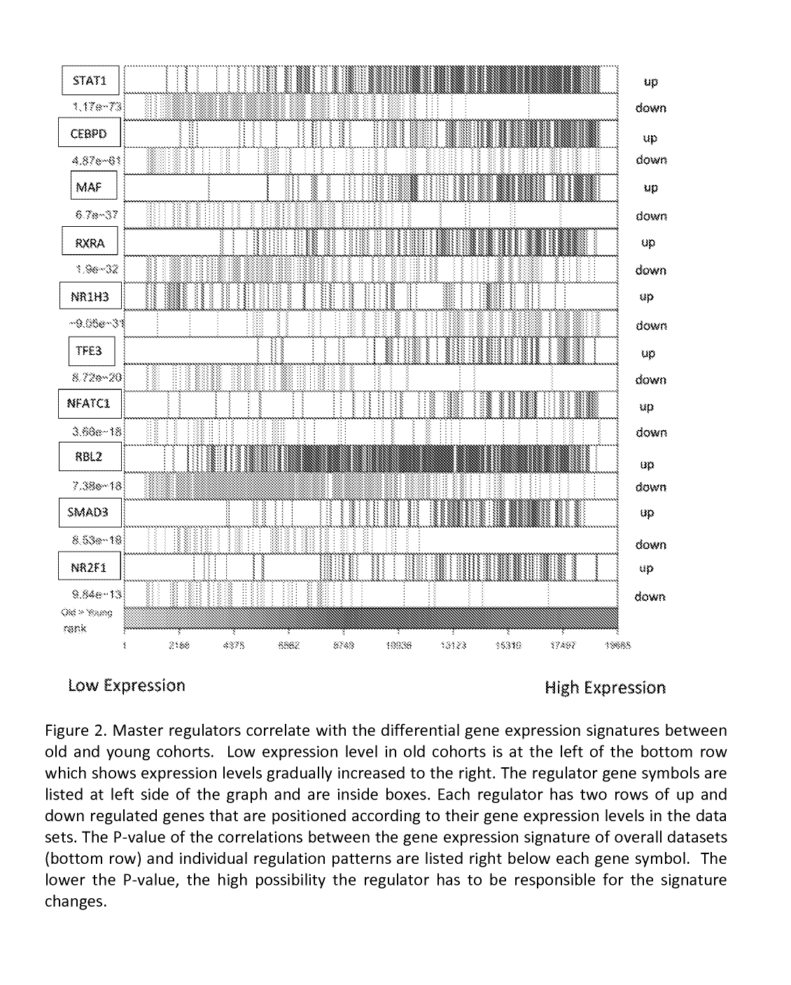

Table 1. This table lists genes of interest with names and symbol and HGNC ID. Table 1 is used to look up the identity of genes listed on Table 4, which shows the degree of up or down-regulation for these genes.

Table 2. This is a list of cellular pathways and other functional categories based on the up- and down-regulated genes in Table 1 showing the degree of enrichment based on the ˜2000 up and down regulated genes. A conservative cut-off with FDR of <=0.1 has been used to identify important pathways.

Table 3. This table shows regulatory genes. This table shows a list of gene signatures and regulatory factors controlling the expression of skin-aging-related genes in various pathways and/or gene networks. Note that the gene described in italics at the end of the table are not part of Table 1 (which only includes the top 2000 up and down regulated genes); these italicized genes are part of the gene network regulators.

Table 4. This table shows the 2000 genes showing the greatest degree of up- or down-regulation and differential expression between the two age cohorts. This table shows the degree of differential expression for the top 2000 up and down regulated genes selected out of over 24000 human genes. ‘Fe’ means Fold Change represented in log 2 scale (these are relative numbers in comparison to expression of other genes so no units are appropriate). The fold-changes in gene expression are all relative to the young subjects. Thus a positive (+) value mean up-regulated in older subject, down-regulated in younger subjects. Negative (−) values means down-regulated in older subjects, up-regulated in younger subjects. HGNC is the standard identification number designated by HUGO Gene Nomenclature Committee. This data was gathered from a total of 32 subjects including 15 samples from the older group (age 59-75 year), and 17 samples from younger group (age 19-21 years). All samples were collected from healthy volunteer white females from an area of the body unexposed to sun (the buttock cheeks). Rest of the procedure are as described herein.

Table 5. This table shows a list of the top 35 genes/regulators controlling the expression of skin-aging-related genes in various genetic networks. This set of genes is selected from the larger list disclosed herein with the intention of supporting specific claims. The italicized genes were discovered during gene network analysis and are not part of Table 1. The inventors explicitly are not restricting the scope of the invention to these 35 genes.

Table 6. This table is a reduced list of the top 23 gene signatures controlling the expression of skin-aging-related genes in genetic networks.

### GENERAL REPRESENTATIONS CONCERNING THE DISCLOSURE

In this specification where reference is made to particular features of the invention it is to be understood that the disclosure of the invention in this specification includes all appropriate combinations of such particular features. The embodiments disclosed in this specification are exemplary and do not limit the invention. As used in this specification, the singular forms “a”, “an”, and “the” include plural reference unless the context clearly dictates otherwise. The term “comprises” and grammatical equivalents thereof are used in this specification to mean that, in addition to the features specifically identified, other features are optionally present. The term “at least” followed by a number is used herein to denote the start of a range beginning with that number. Where reference is made in this specification to a method comprising two or more defined steps, the defined steps can be carried out in any order or simultaneously.

This specification incorporates by reference all documents referred to herein and all documents filed concurrently with this specification or filed previously in connection with this application, including but not limited to such documents which are open to public inspection with this specification. Additionally this application incorporates by reference US provisional application No. 61,198,235 filed Nov. 3, 2008, and also to PCT/US09/063137, international filing date Nov. 3, 2009, and also to the following patents and patent publications: U.S. Pat. No. 6,569,624 US 2004/0142335 US 2006/0275294 US 2003/0152947 US 2005/0089914 US 2007/0059711 US 2005/0250137 US 2005/0250137 US 2005/0053637 U.S. Pat. No. 7,105,292 US 2007/0161022 U.S. Pat. No. 6,692,916 US 2006/0134663 and US 2007/0148106. The specification also incorporates by reference the following: Anuurad, E., Yamasaki, M., Shachter, N., Pearson, T A. and Berglund, L. (2009). ApoE and ApoC-I polymorphisms: association of genotype with cardiovascular disease phenotype in African Americans. J Lipid Res 50(7): 1472-8. Deo, R. C., D. Reich, Tandon, A., Akylbekova, E., Patterson, N., Waliszewska, A., Kathiresan, S., Sarpong, D., Taylor, H A. Jr. and Wilson, J. G. (2009). Genetic differences between the determinants of lipid profile phenotypes in African and European Americans: the Jackson Heart Study. PLoS Genet 5(1): e1000342. Van Dyke, A. L., M. L. Cote, Wenzlaff, A S., Land, S. and Schwartz, AG. (2009). Cytokine SNPs: Comparison of allele frequencies by race and implications for future studies. Cytokine 46(2): 236-44.

### DEFINITIONS

The term “signs of skin aging” refers to any anatomical visible indication that is generally associated with skin as a person gets older, including wrinkles, sagging, discoloration and reduced suppleness.

The term “recalibrating” when applied to the expression of genes, genetic networks, and cellular pathways refers to a change of adjustment of expression of one or more genes to produce a verisimilitude of a former state, such as the adjustment of expression of one or more genes listed in Table 1 so as to increase the production of glycosaminoglycans, proteoglycans, collagen etc.

The term “genetic network” or “genetic pathway” refers to two or more genes the expression of which is coordinated or related to a single physiological function such as the production of a particular protein or glycosaminoglycan.

The term “variant or derivative” when used in conjunction with a species such as a drug or other chemical entity is used to mean said drug or other chemical entity comprising at least one chemical modification, such as, but not limited to, a moiety, a radical group, a reactive group, a charged group, an uncharged group, an ion, or the like. The chemical modification can be either addition or removal of such moiety, group, ion, or the like.

The term “drug” is used to mean any molecule that alters the physiology of an organism.

The term “protein” includes peptides.

The term “environmental stimulus” is used to mean any stimulus that in some way affects the physiology of an organism and that has its origins outside of the organism.

The term “a therapeutic amount” is used to mean an amount (of a substance) that produces a measurable effect related to the health of an organism.

The term “gene expression” is used to refer to the transcription of a gene or a part of a gene and is independent from translation. The expression of the gene or part thereof can be increased or it can be decreased. Translation of the expressed gene or part thereof can be increased or it can be decreased.

The term “gene regulatory network” (GRN) refers to a collection of genes or DNA segments in a cell which interact with each other directly or indirectly through their RNA and protein products. Genes may also interact with other constituents in the cell, thereby governing the rates at which genes in the network are transcribed into mRNA. Some proteins serve only to activate other genes, and these transcription factors that are the main players in regulatory networks or cascades. By binding to the promoter region at the start of other genes they turn them on, initiating the production of another protein, and so on. Some transcription factors are inhibitory.

The term “genetic regulatory module(s)” refers to a gene or group of genes whose action sets the transcription levels for other genes belonging to a network, which satisfy a certain arbitrary threshold criteria, such as 1.5 fold change with a significance p<0.01.

“HGNC” is the standard identification number designated by HUGO Gene Nomenclature Committee (http://www.genenames.org/). The HUGO Gene Nomenclature Committee is responsible for providing human gene naming guidelines and approving new, unique human gene names and symbols (short form abbreviations of full gene name). In addition to species-specific databases, approved gene names and symbols for human can be located in the National Center for Biotechnology Information's Entrez Gene database (http://www.ncbi.nlm.nih.gov/sites/entrez?db=gene). See: Genetic nomenclature guide (1995). Trends Genet and The Trends In Genetics Nomenclature Guide (1998). Elsevier, Cambridge.

### DETAILED DESCRIPTION OF THE INVENTION

The invention relates to expression signatures of genes and to coordinately expressed gene networks associated with skin aging. Additionally the invention relates to methods used to determine the physiological age of skin, and methods used to screen for compounds used to reduce the visible signs of aging of the skin.

The inventors have used high throughput expression screening and SAGE (Serial Analysis of Gene Expression) analysis to compare sets of skin tissue from two cohorts of individuals of substantially different ages. The inventors have discovered a large number of previously unknown expression signatures that are related to age. Additionally they have discovered a large and complex network of coordinately expressed genes and pathways that are associated with he physiological and pathological process of skin aging. The results have identified coordinated gene expression networks and coordination between metabolic pathways that were previously not known to be related to skin ageing. The results have identified a number of transcriptional activators and expression regulators that appear to play key roles in regulation of genes and pathways involved in skin ageing. For example, cholesterol biosynthesis and peroxisome lipid metabolism are both downregulated in the older population, but IL-6 synthesis is upregulated.

The newly discovered expression signatures may be used to (i) determine the physiological age of skin, (ii) screen for compounds and compositions that are used to reduce the visible signs of aging of the skin. Information determined from SAGE analysis includes (1) a categorization of up- and down regulated genes in the younger and older subjects; (2) categorization of physiological and signal transduction pathways in which these genes are over-represented and (3) categorization of gene networks and genes that constitute hubs within the networks.

This disclosure further describes a method for reversing signs of skin aging and risk of skin cancer by recalibrating the expression of genes, genetic networks, and cellular pathways in the human skin, primarily in the dermis, that have changed as a result of the chronological aging process. Gene expression patterns, and the pathways they participate in, are restored to levels characteristic of a younger chronological age by treating the skin with specific combinations of natural compounds (e.g., phyto-chemicals, nutrients, minerals, vitamins, etc). Specific combinations of natural compounds are determined using informatic algorithms and high throughput screening. Natural compounds are delivered to the dermis topically with dermo-cosmetics and internally with oral supplements. Combinations of natural compounds are claimed that produce synergistic effects on the expression of genes relevant to the reversal of skin aging and skin cancer risk reduction. Natural compounds can affect gene expression directly (e.g., transcription factor agonists or antagonist) or indirectly (e.g., non-coding RNAs, epigenetic modifications, signalling receptor agonists or antagonist). The invention includes those natural compounds that produce synergistic effects on gene expression when administered both orally and topically. Also disclosed are those genes, gene networks, non-coding RNAs and epigenetic modifications associated with chronologically younger or older skin.

The invention includes various specific embodiments such as, for example:

A method for delaying, ceasing or reversing signs of skin aging and risk of skin cancer by recalibrating the expression of genes, genetic networks, and cellular pathways in the human skin, primarily in the dermis, that have changed as a result of the chronological aging process wherein the genes are selected from the group consisting of specific genes are listed in Table 1, cellular pathways, and other functional categories are listed in Table 7, and gene signatures controlling the expression of aging-related genes in gene networks in Table 3.

The method above wherein the genes recalibrated comprise one or more genes from Table 1 or genes selected from the group consisting of the genes of the functional categories listed in Table 7 and Table 3.

The method above wherein the genetic networks or cellular pathways recalibrated comprise one or more selected from the groups consisting of the genetic networks or cellular pathways listed in Table 7 and Table 3.

A method for reducing the signs of aging of the skin the method comprising applying to the skin a compound that was identified as having recalibrating potency with the method above.

A method for reducing the signs of aging of the skin the method comprising topically applying to the skin a compound “A” having anti-aging properties and further comprising orally administering a compound “B” having anti-aging properties.

The method above wherein compounds A and B, when administered contemporaneously, provide a synergistic effects on expression of genes of Table 1 or groups of Table 7 and Table 3.

A composition for reducing the signs of aging of the skin, the composition comprising one or more substances that alter the expression of genes involved in the biosynthesis or degradation of a substance selected from the 16 groups listed in Table 7.

A composition for reducing the signs of aging of the skin, the composition comprising one or more substances that increase the expression of genes involved in the biosynthesis of type I or type II collagen.

A composition for reducing the signs of aging of the skin, the composition comprising one or more substances that recalibrate the genes for enzymes listed in Table 1.

A composition for reducing the signs of aging of the skin, the composition comprising one or more substances that decrease production of MMPs.

A composition for reducing the signs of aging of the skin, the composition comprising one or more substances that reduce the rate of degradation of the extracellular matrix proteins in the dermis.

A composition for reducing the signs of aging of the skin, the composition comprising one or more substances that maintain or increase the number of fibroblasts present in the dermis.

A composition for reducing the signs of aging of the skin, the composition comprising one or more substances that maintain or increase the number of collagen fibrils or elastin fibers in the dermis.

A composition for reducing the signs of aging of the skin, the composition comprising one or more substances that maintain or increase the number of collagen fibrils or elastin fibers in the dermis.

A composition for reducing the signs of aging of the skin, the composition comprising one or more substances that maintain or increase the 3-dimensional extracellular matrix structure of collagen, elastin, and other extracellular matrix proteins in the dermis,

A composition for reducing the signs of aging of the skin the composition comprising combinations of natural compounds including phytochemicals, nutrients, minerals, vitamins, etc.

The invention also encompasses compositions of natural compounds, for external application to the skin, that reduce, delay, and/or reverse the signs of aging of the skin; composition of natural compounds, for internal application that reduce, delay, and/or reverse the signs of aging of the skin; compositions of natural compounds that produce synergistic effects on the expression of genes and/or gene products relevant to the reversal of skin aging and skin cancer risk reduction; compositions of natural compounds that affect and/or recalibrate the expression of various sets of genes, genetic networks, and/or cellular pathways in the human skin with the effect of reducing, delaying, and/or reversing the signs of aging of the skin; methods for reducing, delaying, and/or reversing the signs of aging of the skin by the external application and internal administration of claimed compounds; and methods for making above compounds and formulations; and methods for evaluating the efficacy of claimed compounds and formulations.

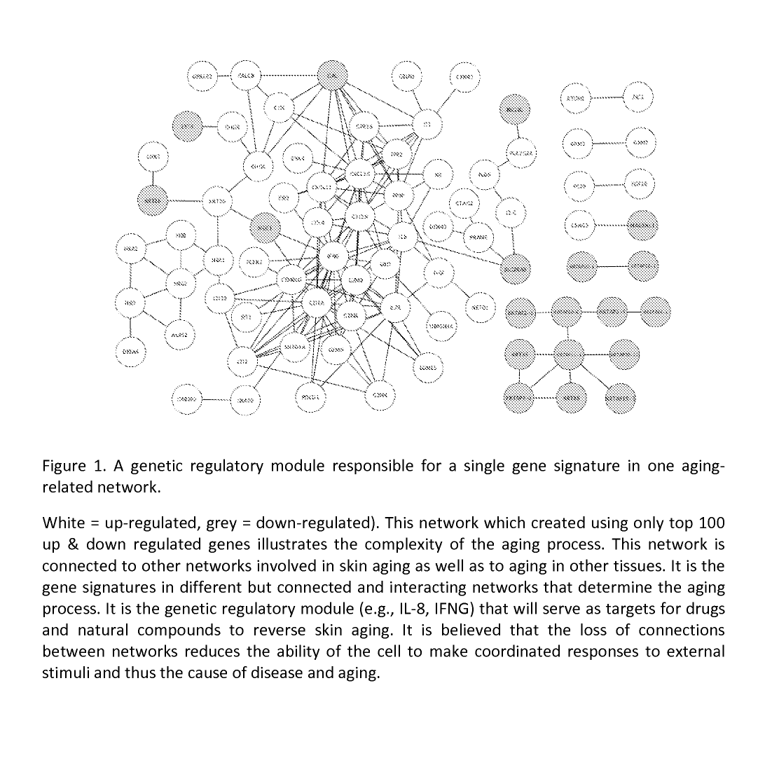

The present work also includes the identification of genetic regulatory modules responsible for gene signatures in aging-related networks. FIG. 1 shows a graphically displayed network (where white=up-regulated, grey=down-regulated genes) showing the complexity of the aging process. This network is connected to other networks involved in skin aging as well as to general aging in other tissues. Key to this and future work is the identification of nodes (genes) that connect networks together. It is the gene signatures in different but connected and interacting networks that determine the aging process. The genetic regulatory module (e.g. IL-6, IL-8, IFNG, CSF3) will serve as targets for drugs and natural compounds to reverse skin aging. The inventors believe that the lose of connections between networks reduces the ability of the cell to make coordinated responses to external stimuli and thus accelerates disease and aging.

The present work also includes the identification of genetic regulatory modules responsible for gene signatures in aging-related networks. FIG. 1 shows a graphically displayed network (where white=up-regulated, grey=down-regulated genes) showing the complexity of the aging process. This network is connected to other networks involved in skin aging as well as to general aging in other tissues. Key to this work is the identification of nodes (genes) that connect networks together. It is the gene signatures in different but connected and interacting networks that determine the aging process. The genetic regulatory module (e.g., IL-6, IL-8, IFNG, CSF3) provide targets for drugs and natural compounds to reverse skin aging. The inventors believe that the loss of connections between networks reduces the ability of the cell to make coordinated responses to external stimuli and thus accelerates disease and aging.

### METHODS OF THE INVENTION

Gene expression analysis was performed on skin samples from a total of 32 subjects including 15 samples from the older group (age 59-75 year), and 17 samples from younger group (age 19-21 years). All samples were collected from healthy volunteer white females from an area of the body unexposed to sun (the white unexposed buttock cheeks).

The following steps were performed:

1) Obtaining of healthy skin from adult female who self-identified themselves as white of different ages.
2) Isolating mRNA from skin tissues for gene expression analysis using microarray technology.
3) Isolating DNA from skin tissues to assess DNA methylation status of key aging-related genes identified in specific aims 6.
4) Performing bioinformatics analysis of the gene expression dataset using various bio-computational and statistical tools.
5) Identification of genes, pathways and networks associated with the skin aging.
6) Identified a small number of genes whose expression can serve as predictors (i.e., biomarkers) of skin aging.
7) Correlation of chronological age with physiological age by use of biomarker genes/gene-net-works.

For data analysis the following steps were carried out.

(1) DNA Sequence fragments from the sequencing experiments were mapped to rna.fa files for Homo Sapiens from NCBI (ftp://ftp.ncbi.nih.gov/genomes/H_sapiens/RNA/, rna.fa.gz, Size: 29793 KB, Last Modified: 11/22/10 7:40:00 PM).

(2) BWA and SAMtools were used to map to sequences in rna.fa file (see item 1) and to count number of fragments match the same sequences with no more than two nucleotide differences. BWA algorithm was published at Li H. and Durbin R. (2009) Fast and accurate short read alignment with Burrows-Wheeler Transform. Bioinformatics, 25:1754-60. (incorporated by reference) and SAMtools was published at Li H.*, Handsaker B.*, Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9. (incorporated by reference).

(3) R package DESeq 1.4.1 was used for statistics analysis. The DESeq algorithm was documented at Anders S. and Huber W. (2010) Differential expression analysis for sequence count data Genome Biology 11:R106 (incorporated by reference).

(4) Network analysis used STRING database. STRING database was published at von Mering C., Huynen M., Jaeggi D., Schmidt S., Bork P., Snel B., (2003), STRING: a database of predicted functional associations between proteins, Nucleic Acids Res. 2003 Jan. 1; 31(1):258-61 (incorporated by reference).

The dermis (inner most layer of the skin) and the epidermis (the outer most layer of the skin) were sampled together in this study. Volunteers, white women at least 18 years of age only, each provided a single, 4 mm deep biopsy of otherwise discarded skin tissue from the buttock.

Exclusion Criteria: Patients with the following criteria were excluded:

individuals with atopic dermatitis, psoriasis and other significant skin disorders

individuals with any serious medical condition

History of alcohol or drug abuse

Known hepatitis or HIV

Pregnant women

Significant allergic or adverse reaction to local anaesthetics

Blood clotting disorder

Faintness or vasovagal reaction with skin biopsies or procedures

Current smoker

The sex, race and health criteria were chosen to exclude as many variants as possible. A homogeneous population was chosen to reduce genetic variables.

Experimental Design

Identification of Genes Differentially Expressed in Healthy Skin from Subjects of Different Chronological Age

Each 4 mm buttock skin sample is immediately immersed into RNALater or snap frozen and stored at −80° C. until nucleic acid (RNA and DNA) extraction.

The samples undergo RNA and DNA extraction.

The extracted RNA is subjected to reverse transcription into cDNA, amplified, and processed for microarray analysis and also by Taqman qRT-PCR.

RNA sequenced are detected by using New Generation Sequencing (NGS), Illumna prpocedure, and data output and analysis is performed per standard analytical software.

Average-linkage hierarchal clustering of data is applied using Gene Cluster software and results are displayed with Java TreeView. Analyses for differential expression include use of appropriate statistical limits for identified gene sets and both paired and multivariate statistical treatment. Gene and pathway identification is assisted by application of interaction gene network analyses for processes such as immune response, inflammation, wound repair, cell growth, apoptosis, cell migration, etc.

This method has lead to pathway-specific hypothesis testing and aids in identification of regulated genes and gene network modules. This treatment of the microarray data enables us to compare gene expression profiles in skin by age.

2. DNA Methylation Status of Aging-Related Gene Signatures.

Based on the results of microarray data analysis, the DNA methylation status of candidate genes that are highly correlated with age (i.e., show the greatest change between young and old skin samples) were determined.

PureLink Genomic DNA Isolation kit (Invitrogen) was used to isolate DNA from half of the nucleic extraction samples.

This genomic DNA was subjected to bisulfite-specific PCR and sequencing of their 5′ promoter regions to determine their DNA methylation status. The DNA is subjected to bisulfite conversion (EZ DNA-Methylation-Direct, Zymo Research).

Bisulfite-converted genomic DNA is subjected to bisulfite-specific PCR (BSP) using primers specific to the 5′ end of the candidate genes. The BSP primers pairs that recognize methylated and unmethylated DNA are designed using Methyl-Primer Express v1.0 (Lifetechnologies).

PCR is carried out in a 25 ul reaction volume using a Taq PCR Kit (New England BioLabs) and PCR products are resolved in 1.5% agarose gels. PCR bands are excised and purified using a Gel Extraction Kit (Qiagen) before ligating to pCR4-TOPO vector (Invitrogen), and transformed into DH5-alpha E. coli. Insert-containing plasmids are verified by PCR and EcoRI digestion before DNA sequencing.

Correlations between DNA hypermethylation in the 5′-ends of aging-related gene signatures, gene expression levels and age are examined. DNA hypermethylation and low level expression of gene signatures in older skin samples are suggestive of epigenetic silencing of gene function, leading to aged skin.

3. Skin Aging Gene Expression (SAGE) Analysis

The initial experiment employed the following samples:

15 samples from the older group (age 59-75 years)
17 samples from younger group (age 19-21 years)
1 Sample has been rejected because of tissue inconsistency
1 Sample has been rejected because of the data quality

Transcript RNA from the samples was isolated and sequenced in 36 bp×36 bp Paired End Reads. This gave about 45M raw reads per sample. About 94% passed quality control. About 84.5% mapped to human transcripts.

Results

The study has discovered a large number of genes and pathways that are differentially expressed between the old and young cohorts.

Additionally the study reveals that many genes and pathways display coordinated expression. This suggests that the coordinated pathways are subject to regulation by means of expression regulators such as transcription regulators, translation regulators, siRNAs and other suppressors of protein expression. Such regulators appear to have pleitropic effects on numerous genes not necessarily linked in series but found at disparate locations in the genome.

The results of the data analysis is shown Tables 1, 2, 3, 4, 5 and 6.

For example, cholesterol biosynthesis and peroxisome lipid metabolism are both downregulated in the older population, but IL-6 synthesis is upregulated.

Another pathway whose regulation is clearly affected by ageing is the T Cell Receptor (TCR) signaling pathway (see Table 7). T Cell Receptor (TCR) activation promotes a number of signaling cascades that ultimately determine cell fate through regulating cytokine production, cell survival, proliferation, and differentiation. An early event in TCR activation is phosphorylation of immunoreceptor tyrosine-base activation motifs (ITAMs) on the cytosolic side of the TCR/CD3 complex by lymphocyte protein-tyrosine kinase (Lck). The CD45 receptor tyrosine phosphatase modulates the phosphorylation and activation of Lck and other Src family tyrosine kinases. z-chain associated protein kinase (Zap-70) is recruited to the TCR/CD3 complex where it becomes activated, promoting recruitment and phosphorylation of downstream adaptor or scaffold proteins. Phosphorylation of SLP-76 by Zap-70 promotes recruitment of Vav (a guanine nucleotide exchange factor), the adaptor proteins NCK and GADS, and an inducible T cell kinase (ITK). Phosphorylation of phospholipase Cγ1 (PLCγ1) by Itk results in the hydrolysis of phosphatidylinositol 4,5-bisphosphate (PIP2) to produce the second messengers diacylglycerol (DAG) and inositol trisphosphate (IP3). DAG activates PKCθ and the MAPK/Erk pathways, both promoting transcription factor NE-KB activation. IP3 triggers the release of Ca2+ from the ER, which promotes the entry of extracellular Ca2+ into cells through calcium release-activated Ca2+(CRAC) channels. Calcium-bound calmodulin (Ca2+/CaM) activates the phosphatase calcineurin, which promotes IL-2 gene transcription through the transcription factor NFAT. Feedback regulation at several points within these pathways allows for different outcomes, depending on the cell type and environment. The incorporation of signals from additional cell surface receptors (such as CD28 or LFA-1) further regulates cellular response.

TABLE 1

| Symbol | Gene Name | HGNC ID |
|---|---|---|
| A2ML1 | alpha-2-macroglobulin-like 1 | HGNC:23336 |
| AACS | acetoacetyl-CoA synthetase | HGNC:21298 |
| AADACL3 | arylacetamide deacetylase-like 3 | HGNC:32037 |
| AADACL4 | arylacetamide deacetylase-like 4 | HGNC:32038 |
| ABCA13 | ATP-binding cassette, sub-family A (ABC1), member 13 | HGNC:14638 |
| ABCA4 | ATP-binding cassette, sub-family A (ABC1), member 4 | HGNC:34 |
| ABCA6 | ATP-binding cassette, sub-family A (ABC1), member 6 | HGNC:36 |
| ABCA8 | ATP-binding cassette, sub-family A (ABC1), member 8 | HGNC:38 |
| ABCA9 | ATP-binding cassette, sub-family A (ABC1), member 9 | HGNC:39 |
| ABCB5 | ATP-binding cassette, sub-family B (MDR/TAP), member | HGNC:46 |
| ABCC11 | ATP-binding cassette, sub-family C (CFTR/MRP), 11 | HGNC:14639 |
| ABCC4 | ATP-binding cassette, sub-family C (CFTR/MRP), 4 | HGNC:55 |
| ABHD12B | abhydrolase domain containing 12B | HGNC:19837 |
| ABHD3 | abhydrolase domain containing 3 | HGNC:18718 |
| ABHD5 | abhydrolase domain containing 5 | HGNC:21396 |
| ABLIM2 | actin binding LIM protein family, member 2 | HGNC:19195 |
| ABLIM3 | actin binding LIM protein family, member 3 | HGNC:29132 |
| ACAA1 | acetyl-CoA acyltransferase 1 | HGNC:82 |
| ACAA2 | acetyl-CoA acyltransferase 2 | HGNC:83 |
| ACAD8 | acyl-CoA dehydrogenase family, member 8 | HGNC:87 |
| ACADM | acyl-CoA dehydrogenase, C-4 to C-12 straight chain | HGNC:89 |
| ACADVL | acyl-CoA dehydrogenase, very long chain | HGNC:92 |
| ACAP1 | ArfGAP with coiled-coil, ankyrin repeat and PH domains1 | HGNC:16467 |
| ACAT2 | acetyl-CoA acetyltransferase 2 | HGNC:94 |
| ACE | angiotensin I converting enzyme (peptidyl-dipeptidase A)1 | HGNC:2707 |
| ACE2 | angiotensin I converting enzyme (peptidyl-dipeptidase A)1 | HGNC:13557 |
| ACLY | ATP citrate lyase | HGNC:115 |
| ACO1 | aconitase 1, soluble | HGNC:117 |
| ACOT1 | acyl-CoA thioesterase 1 | HGNC:33128 |
| ACOT2 | acyl-CoA thioesterase 2 | HGNC:18431 |
| ACOT4 | acyl-CoA thioesterase 4 | HGNC:19748 |
| ACOT7 | acyl-CoA thioesterase 7 | HGNC:24157 |
| ACOX2 | acyl-CoA oxidase 2, branched chain | HGNC:120 |
| ACP6 | acid phosphatase 6, lysophosphatidic | HGNC:29609 |
| ACPP | acid phosphatase, prostate | HGNC:125 |
| ACSBG1 | acyl-CoA synthetase bubblegum family member 1 | HGNC:29567 |
| ACSL1 | acyl-CoA synthetase long-chain family member 1 | HGNC:3569 |
| ACSL3 | acyl-CoA synthetase long-chain family member 3 | HGNC:3570 |
| ACSL5 | acyl-CoA synthetase long-chain family member 5 | HGNC:16526 |
| ACSM3 | acyl-CoA synthetase medium-chain family member 3 | HGNC:10522 |
| ACSM5 | acyl-CoA synthetase medium-chain family member 5 | HGNC:26060 |
| ACSS2 | acyl-CoA synthetase short-chain family member 2 | HGNC:15814 |
| ACTA1 | actin, alpha 1, skeletal muscle | HGNC:129 |
| ACTA2 | actin, alpha 2, smooth muscle, aorta | HGNC:130 |
| ACTBL2 | actin, beta-like 2 | HGNC:17780 |
| ACTC1 | actin, alpha, cardiac muscle 1 | HGNC:143 |
| ACTG2 | actin, gamma 2, smooth muscle, enteric | HGNC:145 |
| ACTN1 | actinin, alpha 1 | HGNC:163 |
| ACTR6 | ARP6 actin-related protein 6 homolog (yeast) | HGNC:24025 |
| ACVR1C | activin A receptor, type IC | HGNC:18123 |
| ACVRL1 | activin A receptor type II-like 1 | HGNC:175 |
| ACY1 | aminoacylase 1 | HGNC:177 |
| ADAM33 | ADAM metallopeptidase domain 33 | HGNC:15478 |
| ADAM8 | ADAM metallopeptidase domain 8 | HGNC:215 |
| ADAMTS15 | ADAM metallopeptidase with thrombospondin type 1 motif, 15 | HGNC:16305 |
| ADAMTS5 | ADAM metallopeptidase with thrombospondin type 1 motif, 5 | HGNC:221 |
| ADAMTS7 | ADAM metallopeptidase with thrombospondin type 1 motif, 7 | HGNC:223 |
| ADAMTSL1 | ADAMTS-like 1 | HGNC:14632 |
| ADAMTSL3 | ADAMTS-like 3 | HGNC:14633 |
| ADAMTSL5 | ADAMTS-like 5 | HGNC:27912 |
| ADCK1 | aarF domain containing kinase 1 | HGNC:19038 |
| ADCY5 | adenylate cyclase 5 | HGNC:236 |
| ADH1A | alcohol dehydrogenase 1A (class I), alpha polypeptide | HGNC:249 |
| ADH1B | alcohol dehydrogenase 1B (class I), beta polypeptide | HGNC:250 |
| ADH1C | alcohol dehydrogenase 1C (class I), gamma polypeptide | HGNC:251 |
| ADH6 | alcohol dehydrogenase 6 (class V) | HGNC:255 |
| ADHFE1 | alcohol dehydrogenase, iron containing, 1 | HGNC:16354 |
| ADIPOQ | adiponectin, C1Q and collagen domain containing | HGNC:13633 |
| ADRA2A | adrenergic, alpha-2A-, receptor | HGNC:281 |
| AEBP1 | AE binding protein 1 | HGNC:303 |
| AGPAT1 | 1-acylglycerol-3-phosphate O-acyltransferase 1 (lysophosphatidic acid | HGNC:324 |
| | acyltransferase, alpha) | |
| AGPAT3 | 1-acylglycerol-3-phosphate O-acyltransferase 3 | HGNC:326 |
| AGPHD1 | aminoglycoside phosphotransferase domain containing 1 | HGNC:34403 |
| AGR2 | anterior gradient 2 homolog (Xenopus laevis) | HGNC:328 |
| AGRN | Agrin | HGNC:329 |
| AGTRAP | angiotensin II receptor-associated protein | HGNC:13539 |
| AHSP | alpha hemoglobin stabilizing protein | HGNC:18075 |
| AKAP12 | A kinase (PRKA) anchor protein 12 | HGNC:370 |
| AKAP2 | A kinase (PRKA) anchor protein 2 | HGNC:372 |
| AKNA | AT-hook transcription factor | HGNC:24108 |
| AKR1A1 | aldo-keto reductase family 1, member A1 (aldehyde reductase) | HGNC:380 |
| ALAS1 | aminolevulinate, delta-, synthase 1 | HGNC:396 |
| ALAS2 | aminolevulinate, delta-, synthase 2 | HGNC:397 |
| ALCAM | activated leukocyte cell adhesion molecule | HGNC:400 |
| ALDH1B1 | aldehyde dehydrogenase 1 family, member B1 | HGNC:407 |
| ALDH1L1 | aldehyde dehydrogenase 1 family, member L1 | HGNC:3978 |
| ALDH3A1 | aldehyde dehydrogenase 3 family, member A1 | HGNC:405 |
| ALDH3B2 | aldehyde dehydrogenase 3 family, member B2 | HGNC:411 |
| ALDH6A1 | aldehyde dehydrogenase 6 family, member A1 | HGNC:7179 |
| ALDOB | aldolase B, fructose-bisphosphate | HGNC:417 |
| ALDOC | aldolase C, fructose-bisphosphate | HGNC:418 |
| ALG1L | asparagine-linked glycosylation 1-like | HGNC:33721 |
| ALOX15B | arachidonate 15-lipoxygenase, type B | HGNC:434 |
| ALPK2 | alpha-kinase 2 | HGNC:20565 |
| AMACR | alpha-methylacyl-CoA racemase | HGNC:451 |
| AMICA1 | adhesion molecule, interacts with CXADR antigen 1 | HGNC:19084 |
| AMOT | angiomotin | HGNC:17810 |
| AMOTL2 | angiomotin like 2 | HGNC:17812 |
| AMPD1 | adenosine monophosphate deaminase 1 | HGNC:468 |
| AMTN | amelotin | HGNC:33188 |
| AMY1A | amylase, alpha 1A (salivary) | HGNC:474 |
| AMY1B | amylase, alpha 1B (salivary) | HGNC:475 |
| AMY1C | amylase, alpha 1C (salivary) | HGNC:476 |
| ANGPT1 | angiopoietin 1 | HGNC:484 |
| ANGPTL2 | angiopoietin-like 2 | HGNC:490 |
| ANGPTL4 | angiopoietin-like 4 | HGNC:16039 |
| ANGPTL5 | angiopoietin-like 5 | HGNC:19705 |
| ANGPTL7 | angiopoietin-like 7 | HGNC:24078 |
| ANKRD1 | ankyrin repeat domain 1 (cardiac muscle) | HGNC:15819 |
| ANKRD22 | ankyrin repeat domain 22 | HGNC:28321 |
| ANKS6 | ankyrin repeat and sterile alpha motif domain containing 6 | HGNC:26724 |
| ANPEP | alanyl (membrane) aminopeptidase | HGNC:500 |
| ANTXR2 | anthrax toxin receptor 2 | HGNC:21732 |
| ANXA3 | annexin A3 | HGNC:541 |
| ANXA6 | annexin A6 | HGNC:544 |
| AOC3 | amine oxidase, copper containing 3 (vascular adhesion protein 1) | HGNC:550 |
| AOX1 | aldehyde oxidase 1 | HGNC:553 |
| APBB1IP | amyloid beta (A4) precursor protein-binding, family B, member 1 interacting | HGNC:17379 |
| | protein | |
| APOBEC3G | apolipoprotein B mRNA editing enzyme, catalytic polypeptide-like 3G | HGNC:17357 |
| APOC1 | apolipoprotein C-I | HGNC:607 |
| APOD | apolipoprotein D | HGNC:612 |
| APOE | apolipoprotein E | HGNC:613 |
| APOL1 | apolipoprotein L, 1 | HGNC:618 |
| APOL3 | apolipoprotein L, 3 | HGNC:14868 |
| APOL6 | apolipoprotein L, 6 | HGNC:14870 |
| AQP4 | aquaporin 4 | HGNC:637 |
| AQP5 | aquaporin 5 | HGNC:638 |
| AQP7 | aquaporin 7 | HGNC:640 |
| AREG | amphiregulin | HGNC:651 |
| AREGB | amphiregulin B | HGNC:34509 |
| ARHGAP20 | Rho GTPase activating protein 20 | HGNC:18357 |
| ARHGAP29 | Rho GTPase activating protein 29 | HGNC:30207 |
| ARHGAP30 | Rho GTPase activating protein 30 | HGNC:27414 |
| ARHGAP6 | Rho GTPase activating protein 6 | HGNC:676 |
| ARHGDIB | Rho GDP dissociation inhibitor (GDI) beta | HGNC:679 |
| ARHGEF6 | Rac/Cdc42 guanine nucleotide exchange factor (GEF) 6 | HGNC:685 |
| ARL6IP1 | ADP-ribosylation factor-like 6 interacting protein 1 | HGNC:697 |
| ARR3 | arrestin 3, retinal (X-arrestin) | HGNC:710 |
| ARSH | arylsulfatase family, member H | HGNC:32488 |
| ASIP | agouti signaling protein | HGNC:745 |
| ASTN1 | astrotactin 1 | HGNC:773 |
| ATOH1 | atonal homolog 1 (Drosophila) | HGNC:797 |
| ATOH8 | atonal homolog 8 (Drosophila) | HGNC:24126 |
| ATP12A | ATPase, H+/K+ transporting, nongastric, alpha polypeptide | HGNC:13816 |
| ATP1A2 | ATPase, Na+/K+ transporting, alpha 2 polypeptide | HGNC:800 |
| ATP5C1 | ATP synthase, H+ transporting, mitochondrial F1 complex, gamma | HGNC:833 |
| | polypeptide 1 | |
| ATP5G3 | ATP synthase, H+ transporting, mitochondrial Fo complex, subunit C3 | HGNC:843 |
| | (subunit 9) | |
| ATP5I | ATP synthase, H+ transporting, mitochondrial Fo complex, subunit E | HGNC:846 |
| ATP6V0A4 | ATPase, H+ transporting, lysosomal V0 subunit a4 | HGNC:866 |
| ATP6V0B | ATPase, H+ transporting, lysosomal 21 kDa, V0 subunit b | HGNC:861 |
| ATP6V0C | ATPase, H+ transporting, lysosomal 16 kDa, V0 subunit c | HGNC:855 |
| ATP6V1C2 | ATPase, H+ transporting, lysosomal 42 kDa, V1 subunit C2 | HGNC:18264 |
| AVIL | advillin | HGNC:14188 |
| AWAT1 | acyl-CoA wax alcohol acyltransferase 1 | HGNC:23252 |
| AWAT2 | acyl-CoA wax alcohol acyltransferase 2 | HGNC:23251 |
| B3GNT3 | UDP-GlcNAc:betaGal beta-1,3-N-acetylglucosaminyltransferase 3 | HGNC:13528 |
| B4GALNT2 | beta-1,4-N-acetyl-galactosaminyl transferase 2 | HGNC:24136 |
| BAI1 | brain-specific angiogenesis inhibitor 1 | HGNC:943 |
| BARX2 | BARX homeobox 2 | HGNC:956 |
| BCAN | brevican | HGNC:23059 |
| BCAP29 | B-cell receptor-associated protein 29 | HGNC:24131 |
| BCAP31 | B-cell receptor-associated protein 31 | HGNC:16695 |
| BCAT1 | branched chain amino-acid transaminase 1, cytosolic | HGNC:976 |
| BCAT2 | branched chain amino-acid transaminase 2, mitochondrial | HGNC:977 |
| BCKDHB | branched chain keto acid dehydrogenase E1, beta polypeptide | HGNC:987 |
| BDH1 | 3-hydroxybutyrate dehydrogenase, type 1 | HGNC:1027 |
| BEAN1 | brain expressed, associated with NEDD4, 1 | HGNC:24160 |
| BHMT2 | betaine--homocysteine S-methyltransferase 2 | HGNC:1048 |
| BICC1 | bicaudal C homolog 1 (Drosophila) | HGNC:19351 |
| BIRC3 | baculoviral IAP repeat containing 3 | HGNC:591 |
| BLNK | B-cell linker | HGNC:14211 |
| BRI3BP | BRI3 binding protein | HGNC:14251 |
| BRP44 | brain protein 44 | HGNC:24515 |
| BRP44L | brain protein 44-like | HGNC:21606 |
| BSG | basigin (Ok blood group) | HGNC:1116 |
| BST1 | bone marrow stromal cell antigen 1 | HGNC:1118 |
| BST2 | bone marrow stromal cell antigen 2 | HGNC:1119 |
| BTN3A1 | butyrophilin, subfamily 3, member A1 | HGNC:1138 |
| BTN3A2 | butyrophilin, subfamily 3, member A2 | HGNC:1139 |
| BTN3A3 | butyrophilin, subfamily 3, member A3 | HGNC:1140 |
| BZW1 | basic leucine zipper and W2 domains 1 | HGNC:18380 |
| C10orf129 | chromosome 10 open reading frame 129 | HGNC:31665 |
| C10orf57 | chromosome 10 open reading frame 57 | HGNC:25804 |
| C11orf96 | chromosome 11 open reading frame 96 | HGNC:38675 |
| C12orf39 | chromosome 12 open reading frame 39 | HGNC:28139 |
| C14orf180 | chromosome 14 open reading frame 180 | HGNC:33795 |
| C14orf2 | chromosome 14 open reading frame 2 | HGNC:1188 |
| C14orf49 | chromosome 14 open reading frame 49 | HGNC:19861 |
| C15orf23 | chromosome 15 open reading frame 23 | HGNC:30767 |
| C15orf43 | chromosome 15 open reading frame 43 | HGNC:28520 |
| C15orf48 | chromosome 15 open reading frame 48 | HGNC:29898 |
| C16orf73 | chromosome 16 open reading frame 73 | HGNC:28569 |
| C16orf74 | chromosome 16 open reading frame 74 | HGNC:23362 |
| C16orf89 | chromosome 16 open reading frame 89 | HGNC:28687 |
| C17orf97 | chromosome 17 open reading frame 97 | HGNC:33800 |
| C19orf46 | chromosome 19 open reading frame 46 | HGNC:26703 |
| C19orf71 | chromosome 19 open reading frame 71 | HGNC:34496 |
| C1orf122 | chromosome 1 open reading frame 122 | HGNC:24789 |
| C1orf158 | chromosome 1 open reading frame 158 | HGNC:28567 |
| C1orf162 | chromosome 1 open reading frame 162 | HGNC:28344 |
| C1orf51 | chromosome 1 open reading frame 51 | HGNC:25200 |
| C1orf53 | chromosome 1 open reading frame 53 | HGNC:30003 |
| C1orf68 | chromosome 1 open reading frame 68 | HGNC:29468 |
| C1orf9 | chromosome 1 open reading frame 9 | HGNC:1240 |
| C1QA | complement component 1, q subcomponent, A chain | HGNC:1241 |
| C1QB | complement component 1, q subcomponent, B chain | HGNC:1242 |
| C1QC | complement component 1, q subcomponent, C chain | HGNC:1245 |
| C1QTNF5 | C1q and tumor necrosis factor related protein 5 | HGNC:14344 |
| C1QTNF6 | C1q and tumor necrosis factor related protein 6 | HGNC:14343 |
| C1R | complement component 1, r subcomponent | HGNC:1246 |
| C1S | complement component 1, s subcomponent | HGNC:1247 |
| C2 | complement component 2 | HGNC:1248 |
| C20orf24 | chromosome 20 open reading frame 24 | HGNC:15870 |
| C20orf3 | chromosome 20 open reading frame 3 | HGNC:13238 |
| C21orf33 | chromosome 21 open reading frame 33 | HGNC:1273 |
| C3 | complement component 3 | HGNC:1318 |
| C3AR1 | complement component 3a receptor 1 | HGNC:1319 |
| C5orf4 | chromosome 5 open reading frame 4 | HGNC:1334 |
| C6orf106 | chromosome 6 open reading frame 106 | HGNC:21215 |
| C7 | complement component 7 | HGNC:1346 |
| C7orf58 | chromosome 7 open reading frame 58 | HGNC:26159 |
| C9orf16 | chromosome 9 open reading frame 16 | HGNC:17823 |
| CA14 | carbonic anhydrase XIV | HGNC:1372 |
| CA6 | carbonic anhydrase VI | HGNC:1380 |
| CALCB | calcitonin-related polypeptide beta | HGNC:1438 |
| CALD1 | caldesmon 1 | HGNC:1441 |
| CALML3 | calmodulin-like 3 | HGNC:1452 |
| CAMK1G | calcium/calmodulin-dependent protein kinase IG | HGNC:14585 |
| CAMP | cathelicidin antimicrobial peptide | HGNC:1472 |
| CAPN13 | calpain 13 | HGNC:16663 |
| CAPN3 | calpain 3, (p94) | HGNC:1480 |
| CAPN8 | calpain 8 | HGNC:1485 |
| CARD11 | caspase recruitment domain family, member 11 | HGNC:16393 |
| CARD18 | caspase recruitment domain family, member 18 | HGNC:28861 |
| CASQ2 | calsequestrin 2 (cardiac muscle) | HGNC:1513 |
| CASS4 | Cas scaffolding protein family member 4 | HGNC:15878 |
| CBLN1 | cerebellin 1 precursor | HGNC:1543 |
| CBLN2 | cerebellin 2 precursor | HGNC:1544 |
| CCBP2 | chemokine binding protein 2 | HGNC:1565 |
| CCDC69 | coiled-coil domain containing 69 | HGNC:24487 |
| CCDC85A | coiled-coil domain containing 85A | HGNC:29400 |
| CCDC86 | coiled-coil domain containing 86 | HGNC:28359 |
| CCK | cholecystokinin | HGNC:1569 |
| CCKBR | cholecystokinin B receptor | HGNC:1571 |
| CCL13 | chemokine (C-C motif) ligand 13 | HGNC:10611 |
| CCL14 | chemokine (C-C motif) ligand 14 | HGNC:10612 |
| CCL15 | chemokine (C-C motif) ligand 15 | HGNC:10613 |
| CCL17 | chemokine (C-C motif) ligand 17 | HGNC:10615 |
| CCL18 | chemokine (C-C motif) ligand 18 (pulmonary and activation-regulated) | HGNC:10616 |
| CCL19 | chemokine (C-C motif) ligand 19 | HGNC:10617 |
| CCL27 | chemokine (C-C motif) ligand 27 | HGNC:10626 |
| CCL4 | chemokine (C-C motif) ligand 4 | HGNC:10630 |
| CCL4L2 | chemokine (C-C motif) ligand 4-like 2 | HGNC:24066 |
| CCL5 | chemokine (C-C motif) ligand 5 | HGNC:10632 |
| CCNC | cyclin C | HGNC:1581 |
| CCND1 | cyclin D1 | HGNC:1582 |
| CCNF | cyclin F | HGNC:1591 |
| CCR1 | chemokine (C-C motif) receptor 1 | HGNC:1602 |
| CCR2 | chemokine (C-C motif) receptor 2 | HGNC:1603 |
| CCR4 | chemokine (C-C motif) receptor 4 | HGNC:1605 |
| CCR5 | chemokine (C-C motif) receptor 5 (gene/pseudogene) | HGNC:1606 |
| CCR7 | chemokine (C-C motif) receptor 7 | HGNC:1608 |
| CD163 | CD163 molecule | HGNC:1631 |
| CD1C | CD1c molecule | HGNC:1636 |
| CD1E | CD1e molecule | HGNC:1638 |
| CD2 | CD2 molecule | HGNC:1639 |
| CD207 | CD207 molecule, langerin | HGNC:17935 |
| CD209 | CD209 molecule | HGNC:1641 |
| CD226 | CD226 molecule | HGNC:16961 |
| CD24 | CD24 molecule | HGNC:1645 |
| CD247 | CD247 molecule | HGNC:1677 |
| CD248 | CD248 molecule, endosialin | HGNC:18219 |
| CD27 | CD27 molecule | HGNC:11922 |
| CD28 | CD28 molecule | HGNC:1653 |
| CD34 | CD34 molecule | HGNC:1662 |
| CD36 | CD36 molecule (thrombospondin receptor) | HGNC:1663 |
| CD37 | CD37 molecule | HGNC:1666 |
| CD38 | CD38 molecule | HGNC:1667 |
| CD3D | CD3d molecule, delta (CD3-TCR complex) | HGNC:1673 |
| CD3E | CD3e molecule, epsilon (CD3-TCR complex) | HGNC:1674 |
| CD40LG | CD40 ligand | HGNC:11935 |
| CD48 | CD48 molecule | HGNC:1683 |
| CD5 | CD5 molecule | HGNC:1685 |
| CD52 | CD52 molecule | HGNC:1804 |
| CD53 | CD53 molecule | HGNC:1686 |
| CD6 | CD6 molecule | HGNC:1691 |
| CD68 | CD68 molecule | HGNC:1693 |
| CD74 | CD74 molecule, major histocompatibility complex, class II invariant | HGNC:1697 |
| CD84 | CD84 molecule | HGNC:1704 |
| CD8A | CD8a molecule | HGNC:1706 |
| CD96 | CD96 molecule | HGNC:16892 |
| CD97 | CD97 molecule | HGNC:1711 |
| CDA | cytidine deaminase | HGNC:1712 |
| CDH5 | cadherin 5, type 2 (vascular endothelium) | HGNC:1764 |
| CDH7 | cadherin 7, type 2 | HGNC:1766 |
| CDK5 | cyclin-dependent kinase 5 | HGNC:1774 |
| CDKL2 | cyclin-dependent kinase-like 2 (CDC2-related kinase) | HGNC:1782 |
| CDKN1A | cyclin-dependent kinase inhibitor 1A (p21, Cip1) | HGNC:1784 |
| CDO1 | cysteine dioxygenase, type I | HGNC:1795 |
| CEACAM1 | carcinoembryonic antigen-related cell adhesion molecule 1 | HGNC:1814 |
| CEACAM6 | carcinoembryonic antigen-related cell adhesion molecule 6 | HGNC:1818 |
| CECR2 | cat eye syndrome chromosome region, candidate 2 | HGNC:1840 |
| CELF2 | CUGBP, Elav-like family member 2 | HGNC:2550 |
| CES1 | carboxylesterase 1 | HGNC:1863 |
| CFB | complement factor B | HGNC:1037 |
| CFD | complement factor D (adipsin) | HGNC:2771 |
| CFH | complement factor H | HGNC:4883 |
| CFHR1 | complement factor H-related 1 | HGNC:4888 |
| CFHR3 | complement factor H-related 3 | HGNC:16980 |
| CGA | glycoprotein hormones, alpha polypeptide | HGNC:1885 |
| CHCHD10 | coiled-coil-helix-coiled-coil-helix domain containing 10 | HGNC:15559 |
| CHGA | chromogranin A (parathyroid secretory protein 1) | HGNC:1929 |
| CHGB | chromogranin B (secretogranin 1) | HGNC:1930 |
| CHI3L1 | chitinase 3-like 1 (cartilage glycoprotein-39) | HGNC:1932 |
| CHI3L2 | chitinase 3-like 2 | HGNC:1933 |
| CHIT1 | chitinase 1 (chitotriosidase) | HGNC:1936 |
| CHL1 | cell adhesion molecule with homology to L1CAM | HGNC:1939 |
| CHRDL1 | chordin-like 1 | HGNC:29861 |
| CHRDL2 | chordin-like 2 | HGNC:24168 |
| CHRNA9 | cholinergic receptor, nicotinic, alpha 9 (neuronal) | HGNC:14079 |
| CHST2 | carbohydrate (N-acetylglucosamine-6-O) sulfotransferase 2 | HGNC:1970 |
| CIDEA | cell death-inducing DFFA-like effector a | HGNC:1976 |
| CIDEC | cell death-inducing DFFA-like effector c | HGNC:24229 |
| CILP | cartilage intermediate layer protein, nucleotide pyrophosphohydrolase | HGNC:1980 |
| CILP2 | cartilage intermediate layer protein 2 | HGNC:24213 |
| CISH | cytokine inducible SH2-containing protein | HGNC:1984 |
| CKB | creatine kinase, brain | HGNC:1991 |
| CKMT1A | creatine kinase, mitochondrial 1A | HGNC:31736 |
| CKMT1B | creatine kinase, mitochondrial 1B | HGNC:1995 |
| CLC | Charcot-Leyden crystal protein | HGNC:2014 |
| CLCA2 | chloride channel accessory 2 | HGNC:2016 |
| CLDN10 | claudin 10 | HGNC:2033 |
| CLDN11 | claudin 11 | HGNC:8514 |
| CLDN5 | claudin 5 | HGNC:2047 |
| CLEC10A | C-type lectin domain family 10, member A | HGNC:16916 |
| CLEC12A | C-type lectin domain family 12, member A | HGNC:31713 |
| CLEC2A | C-type lectin domain family 2, member A | HGNC:24191 |
| CLEC3B | C-type lectin domain family 3, member B | HGNC:11891 |
| CLEC4G | C-type lectin domain family 4, member G | HGNC:24591 |
| CLIC2 | chloride intracellular channel 2 | HGNC:2063 |
| CLIC3 | chloride intracellular channel 3 | HGNC:2064 |
| CLIC6 | chloride intracellular channel 6 | HGNC:2065 |
| CLIP2 | CAP-GLY domain containing linker protein 2 | HGNC:2586 |
| CLIP3 | CAP-GLY domain containing linker protein 3 | HGNC:24314 |
| CLSTN3 | calsyntenin 3 | HGNC:18371 |
| CLU | clusterin | HGNC:2095 |
| CMA1 | chymase 1, mast cell | HGNC:2097 |
| CMTM8 | CKLF-like MARVEL transmembrane domain containing 8 | HGNC:19179 |
| CNDP1 | carnosine dipeptidase 1 (metallopeptidase M20 family) | HGNC:20675 |
| CNDP2 | CNDP dipeptidase 2 (metallopeptidase M20 family) | HGNC:24437 |
| CNIH4 | cornichon homolog 4 (Drosophila) | HGNC:25013 |
| CNKSR3 | CNKSR family member 3 | HGNC:23034 |
| CNN1 | calponin 1, basic, smooth muscle | HGNC:2155 |
| CNPY1 | canopy 1 homolog (zebrafish) | HGNC:27786 |
| COCH | coagulation factor C homolog, cochlin (Limulus polyphemus) | HGNC:2180 |
| COL11A1 | collagen, type XI, alpha 1 | HGNC:2186 |
| COL12A1 | collagen, type XII, alpha 1 | HGNC:2188 |
| COL16A1 | collagen, type XVI, alpha 1 | HGNC:2193 |
| COL1A1 | collagen, type I, alpha 1 | HGNC:2197 |
| COL1A2 | collagen, type I, alpha 2 | HGNC:2198 |
| COL3A1 | collagen, type III, alpha 1 | HGNC:2201 |
| COL4A1 | collagen, type IV, alpha 1 | HGNC:2202 |
| COL4A2 | collagen, type IV, alpha 2 | HGNC:2203 |
| COL4A6 | collagen, type IV, alpha 6 | HGNC:2208 |
| COL5A1 | collagen, type V, alpha 1 | HGNC:2209 |
| COL7A1 | collagen, type VII, alpha 1 | HGNC:2214 |
| COL8A1 | collagen, type VIII, alpha 1 | HGNC:2215 |
| COMP | cartilage oligomeric matrix protein | HGNC:2227 |
| COQ4 | coenzyme Q4 homolog (S. cerevisiae) | HGNC:19693 |
| COQ9 | coenzyme Q9 homolog (S. cerevisiae) | HGNC:25302 |
| CORO1A | coronin, actin binding protein, 1A | HGNC:2252 |
| COTL1 | coactosin-like 1 (Dictyostelium) | HGNC:18304 |
| COX5A | cytochrome c oxidase subunit Va | HGNC:2267 |
| COX6C | cytochrome c oxidase subunit VIc | HGNC:2285 |
| COX7A2 | cytochrome c oxidase subunit VIIa polypeptide 2 (liver) | HGNC:2288 |
| COX7B | cytochrome c oxidase subunit VIIb | HGNC:2291 |
| COX7C | cytochrome c oxidase subunit VIIc | HGNC:2292 |
| COX8A | cytochrome c oxidase subunit VIIIA (ubiquitous) | HGNC:2294 |
| CP | ceruloplasmin (ferroxidase) | HGNC:2295 |
| CPA3 | carboxypeptidase A3 (mast cell) | HGNC:2298 |
| CPA4 | carboxypeptidase A4 | HGNC:15740 |
| CPB2 | carboxypeptidase B2 (plasma) | HGNC:2300 |
| CPD | carboxypeptidase D | HGNC:2301 |
| CPE | carboxypeptidase E | HGNC:2303 |
| CPEB4 | cytoplasmic polyadenylation element binding protein 4 | HGNC:21747 |
| CPLX3 | complexin 3 | HGNC:27652 |
| CPT2 | carnitine palmitoyltransferase 2 | HGNC:2330 |
| CPVL | carboxypeptidase, vitellogenic-like | HGNC:14399 |
| CPXM1 | carboxypeptidase X (M14 family), member 1 | HGNC:15771 |
| CPZ | carboxypeptidase Z | HGNC:2333 |
| CRABP1 | cellular retinoic acid binding protein 1 | HGNC:2338 |
| CRAT | carnitine O-acetyltransferase | HGNC:2342 |
| CREG1 | cellular repressor of E1A-stimulated genes 1 | HGNC:2351 |
| CRISP3 | cysteine-rich secretory protein 3 | HGNC:16904 |
| CRYAB | crystallin, alpha B | HGNC:2389 |
| CRYBB2 | crystallin, beta B2 | HGNC:2398 |
| CS | citrate synthase | HGNC:2422 |
| CSAG3 | CSAG family, member 3 | HGNC:26237 |
| CSF1 | colony stimulating factor 1 (macrophage) | HGNC:2432 |
| CSF1R | colony stimulating factor 1 receptor | HGNC:2433 |
| CSN1S1 | casein alpha s1 | HGNC:2445 |
| CSPG4 | chondroitin sulfate proteoglycan 4 | HGNC:2466 |
| CSRP2 | cysteine and glycine-rich protein 2 | HGNC:2470 |
| CST1 | cystatin SN | HGNC:2473 |
| CST6 | cystatin E/M | HGNC:2478 |
| CST7 | cystatin F (leukocystatin) | HGNC:2479 |
| CSTB | cystatin B (stefin B) | HGNC:2482 |
| CTAG2 | cancer/testis antigen 2 | HGNC:2492 |
| CTH | cystathionase (cystathionine gamma-lyase) | HGNC:2501 |
| CTHRC1 | collagen triple helix repeat containing 1 | HGNC:18831 |
| CTNND2 | catenin (cadherin-associated protein), delta 2 | HGNC:2516 |
| CTSG | cathepsin G | HGNC:2532 |
| CTSK | cathepsin K | HGNC:2536 |
| CTSL2 | cathepsin L2 | HGNC:2538 |
| CTSS | cathepsin S | HGNC:2545 |
| CUBN | cubilin (intrinsic factor-cobalamin receptor) | HGNC:2548 |
| CUX2 | cut-like homeobox 2 | HGNC:19347 |
| CXCL10 | chemokine (C—X—C motif) ligand 10 | HGNC:10637 |
| CXCL11 | chemokine (C—X—C motif) ligand 11 | HGNC:10638 |
| CXCL9 | chemokine (C—X—C motif) ligand 9 | HGNC:7098 |
| CXCR3 | chemokine (C—X—C motif) receptor 3 | HGNC:4540 |
| CXCR7 | chemokine (C—X—C motif) receptor 7 | HGNC:23692 |
| CYB5A | cytochrome b5 type A (microsomal) | HGNC:2570 |
| CYBB | cytochrome b-245, beta polypeptide | HGNC:2578 |
| CYBRD1 | cytochrome b reductase 1 | HGNC:20797 |
| CYCS | cytochrome c, somatic | HGNC:19986 |
| CYP19A1 | cytochrome P450, family 19, subfamily A, polypeptide 1 | HGNC:2594 |
| CYP1A1 | cytochrome P450, family 1, subfamily A, polypeptide 1 | HGNC:2595 |
| CYP1A2 | cytochrome P450, family 1, subfamily A, polypeptide 2 | HGNC:2596 |
| CYP1B1 | cytochrome P450, family 1, subfamily B, polypeptide 1 | HGNC:2597 |
| CYP26B1 | cytochrome P450, family 26, subfamily B, polypeptide 1 | HGNC:20581 |
| CYP27A1 | cytochrome P450, family 27, subfamily A, polypeptide 1 | HGNC:2605 |
| CYP27B1 | cytochrome P450, family 27, subfamily B, polypeptide 1 | HGNC:2606 |
| CYP2R1 | cytochrome P450, family 2, subfamily R, polypeptide 1 | HGNC:20580 |
| CYP3A4 | cytochrome P450, family 3, subfamily A, polypeptide 4 | HGNC:2637 |
| CYP4B1 | cytochrome P450, family 4, subfamily B, polypeptide 1 | HGNC:2644 |
| CYP4F2 | cytochrome P450, family 4, subfamily F, polypeptide 2 | HGNC:2645 |
| CYP4F22 | cytochrome P450, family 4, subfamily F, polypeptide 22 | HGNC:26820 |
| CYP4F3 | cytochrome P450, family 4, subfamily F, polypeptide 3 | HGNC:2646 |
| CYP4F8 | cytochrome P450, family 4, subfamily F, polypeptide 8 | HGNC:2648 |
| CYP51A1 | cytochrome P450, family 51, subfamily A, polypeptide 1 | HGNC:2649 |
| CYTH4 | cytohesin 4 | HGNC:9505 |
| DACH1 | dachshund homolog 1 (Drosophila) | HGNC:2663 |
| DAPK2 | death-associated protein kinase 2 | HGNC:2675 |
| DBT | dihydrolipoamide branched chain transacylase E2 | HGNC:2698 |
| DCD | dermcidin | HGNC:14669 |
| DCT | dopachrome tautomerase | HGNC:2709 |
| DCXR | dicarbonyl/L-xylulose reductase | HGNC:18985 |
| DDC | dopa decarboxylase (aromatic L-amino acid decarboxylase) | HGNC:2719 |
| DDHD1 | DDHD domain containing 1 | HGNC:19714 |
| DDIT4 | DNA-damage-inducible transcript 4 | HGNC:24944 |
| DDO | D-aspartate oxidase | HGNC:2727 |
| DDR2 | discoidin domain receptor tyrosine kinase 2 | HGNC:2731 |
| DDX43 | DEAD (Asp-Glu-Ala-Asp) box polypeptide 43 | HGNC:18677 |
| DDX60 | DEAD (Asp-Glu-Ala-Asp) box polypeptide 60 | HGNC:25942 |
| DEFA6 | defensin, alpha 6, Paneth cell-specific | HGNC:2765 |
| DEFB132 | defensin, beta 132 | HGNC:33806 |
| DENND3 | DENN/MADD domain containing 3 | HGNC:29134 |
| DES | desmin | HGNC:2770 |
| DGAT2 | diacylglycerol O-acyltransferase 2 | HGNC:16940 |
| DGAT2L6 | diacylglycerol O-acyltransferase 2-like 6 | HGNC:23250 |
| DHCR24 | 24-dehydrocholesterol reductase | HGNC:2859 |
| DHCR7 | 7-dehydrocholesterol reductase | HGNC:2860 |
| DHRS11 | dehydrogenase/reductase (SDR family) member 11 | HGNC:28639 |
| DHRS2 | dehydrogenase/reductase (SDR family) member 2 | HGNC:18349 |
| DHRS7B | dehydrogenase/reductase (SDR family) member 7B | HGNC:24547 |
| DHRS9 | dehydrogenase/reductase (SDR family) member 9 | HGNC:16888 |
| DIO2 | deiodinase, iodothyronine, type II | HGNC:2884 |
| DIRAS1 | DIRAS family, GTP-binding RAS-like 1 | HGNC:19127 |
| DIRAS3 | DIRAS family, GTP-binding RAS-like 3 | HGNC:687 |
| DKK1 | dickkopf 1 homolog (Xenopus laevis) | HGNC:2891 |
| DLD | dihydrolipoamide dehydrogenase | HGNC:2898 |
| DLK1 | delta-like 1 homolog (Drosophila) | HGNC:2907 |
| DMBX1 | diencephalon/mesencephalon homeobox 1 | HGNC:19026 |
| DMRTA1 | DMRT-like family A1 | HGNC:13826 |
| DNAH17 | dynein, axonemal, heavy chain 17 | HGNC:2946 |
| DNAH8 | dynein, axonemal, heavy chain 8 | HGNC:2952 |
| DNASE1L2 | deoxyribonuclease I-like 2 | HGNC:2958 |
| DOC2B | double C2-like domains, beta | HGNC:2986 |
| DOCK11 | dedicator of cytokinesis 11 | HGNC:23483 |
| DOCK2 | dedicator of cytokinesis 2 | HGNC:2988 |
| DOK2 | docking protein 2, 56 kDa | HGNC:2991 |
| DOPEY2 | dopey family member 2 | HGNC:1291 |
| DPEP1 | dipeptidase 1 (renal) | HGNC:3002 |
| DPP4 | dipeptidyl-peptidase 4 | HGNC:3009 |
| DPT | dermatopontin | HGNC:3011 |
| DPYD | dihydropyrimidine dehydrogenase | HGNC:3012 |
| DRAM1 | DNA-damage regulated autophagy modulator 1 | HGNC:25645 |
| DSC2 | desmocollin 2 | HGNC:3036 |
| DSE | dermatan sulfate epimerase | HGNC:21144 |
| DSG4 | desmoglein 4 | HGNC:21307 |
| DTNA | dystrobrevin, alpha | HGNC:3057 |
| DTX4 | deltex homolog 4 (Drosophila) | HGNC:29151 |
| DUSP1 | dual specificity phosphatase 1 | HGNC:3064 |
| DUSP16 | dual specificity phosphatase 16 | HGNC:17909 |
| DUSP4 | dual specificity phosphatase 4 | HGNC:3070 |
| DUSP5 | dual specificity phosphatase 5 | HGNC:3071 |
| DUSP6 | dual specificity phosphatase 6 | HGNC:3072 |
| EBF1 | early B-cell factor 1 | HGNC:3126 |
| EBI3 | Epstein-Barr virus induced 3 | HGNC:3129 |
| EBP | emopamil binding protein (sterol isomerase) | HGNC:3133 |
| ECH1 | enoyl CoA hydratase 1, peroxisomal | HGNC:3149 |
| ECHDC1 | enoyl CoA hydratase domain containing 1 | HGNC:21489 |
| ECHDC2 | enoyl CoA hydratase domain containing 2 | HGNC:23408 |
| ECM1 | extracellular matrix protein 1 | HGNC:3153 |
| ECM2 | extracellular matrix protein 2, female organ and adipocyte specific | HGNC:3154 |
| EDA2R | ectodysplasin A2 receptor | HGNC:17756 |
| EDDM3B | epididymal protein 3B | HGNC:19223 |
| EFCAB4A | EF-hand calcium binding domain 4A | HGNC:28703 |
| EFEMP1 | EGF containing fibulin-like extracellular matrix protein 1 | HGNC:3218 |
| EFNB2 | ephrin-B2 | HGNC:3227 |
| EGFL6 | EGF-like-domain, multiple 6 | HGNC:3235 |
| EGLN3 | egl nine homolog 3 (C. elegans) | HGNC:14661 |
| EGR1 | early growth response 1 | HGNC:3238 |
| EGR2 | early growth response 2 | HGNC:3239 |
| EGR3 | early growth response 3 | HGNC:3240 |
| EHD2 | EH-domain containing 2 | HGNC:3243 |
| EHF | ets homologous factor | HGNC:3246 |
| EI24 | etoposide induced 2.4 mRNA | HGNC:13276 |
| ELANE | elastase, neutrophil expressed | HGNC:3309 |
| ELAVL2 | ELAV (embryonic lethal, abnormal vision, Drosophila)-like 2 | HGNC:3313 |
| ELL2 | elongation factor, RNA polymerase II, 2 | HGNC:17064 |
| ELOVL1 | ELOVL fatty acid elongase 1 | HGNC:14418 |
| ELOVL3 | ELOVL fatty acid elongase 3 | HGNC:18047 |
| ELOVL4 | ELOVL fatty acid elongase 4 | HGNC:14415 |
| ELOVL5 | ELOVL fatty acid elongase 5 | HGNC:21308 |
| EMID2 | EMI domain containing 2 | HGNC:18038 |
| EMILIN1 | elastin microfibril interfacer 1 | HGNC:19880 |
| EMILIN2 | elastin microfibril interfacer 2 | HGNC:19881 |
| EMILIN3 | elastin microfibril interfacer 3 | HGNC:16123 |
| EML6 | echinoderm microtubule associated protein like 6 | HGNC:35412 |
| EN2 | engrailed homeobox 2 | HGNC:3343 |
| ENC1 | ectodermal-neural cortex 1 (with BTB-like domain) | HGNC:3345 |
| ENDOU | endonuclease, polyU-specific | HGNC:14369 |
| ENG | endoglin | HGNC:3349 |
| ENO2 | enolase 2 (gamma, neuronal) | HGNC:3353 |
| EOMES | eomesodermin | HGNC:3372 |
| EPB41L2 | erythrocyte membrane protein band 4.1-like 2 | HGNC:3379 |
| EPPK1 | epiplakin 1 | HGNC:15577 |
| ERAP1 | endoplasmic reticulum aminopeptidase 1 | HGNC:18173 |
| ESRRA | estrogen-related receptor alpha | HGNC:3471 |
| ETFDH | electron-transferring-flavoprotein dehydrogenase | HGNC:3483 |
| ETHE1 | ethylmalonic encephalopathy 1 | HGNC:23287 |
| ETV4 | ets variant 4 | HGNC:3493 |
| EVI2B | ecotropic viral integration site 2B | HGNC:3500 |
| EYA4 | eyes absent homolog 4 (Drosophila) | HGNC:3522 |
| F10 | coagulation factor X | HGNC:3528 |
| F13A1 | coagulation factor XIII, A1 polypeptide | HGNC:3531 |
| F3 | coagulation factor III (thromboplastin, tissue factor) | HGNC:3541 |
| F9 | coagulation factor IX | HGNC:3551 |
| FA2H | fatty acid 2-hydroxylase | HGNC:21197 |
| FABP12 | fatty acid binding protein 12 | HGNC:34524 |
| FABP4 | fatty acid binding protein 4, adipocyte | HGNC:3559 |
| FABP6 | fatty acid binding protein 6, ileal | HGNC:3561 |
| FABP7 | fatty acid binding protein 7, brain | HGNC:3562 |
| FABP9 | fatty acid binding protein 9, testis | HGNC:3563 |
| FADS1 | fatty acid desaturase 1 | HGNC:3574 |
| FADS2 | fatty acid desaturase 2 | HGNC:3575 |
| FADS6 | fatty acid desaturase domain family, member 6 | HGNC:30459 |
| FAM108C1 | family with sequence similarity 108, member C1 | HGNC:26925 |
| FAM126A | family with sequence similarity 126, member A | HGNC:24587 |
| FAM129A | family with sequence similarity 129, member A | HGNC:16784 |
| FAM132A | family with sequence similarity 132, member A | HGNC:32308 |
| FAM150B | family with sequence similarity 150, member B | HGNC:27683 |
| FAM176A | family with sequence similarity 176, member A | HGNC:25816 |
| FAM181A | family with sequence similarity 181, member A | HGNC:20491 |
| FAM196B | family with sequence similarity 196, member B | HGNC:37271 |
| FAM19A2 | family with sequence similarity 19 (chemokine (C-C motif)-like), member A2 | HGNC:21589 |
| FAM26D | family with sequence similarity 26, member D | HGNC:21094 |
| FAM26E | family with sequence similarity 26, member E | HGNC:21568 |
| FAM3C | family with sequence similarity 3, member C | HGNC:18664 |
| FAM3D | family with sequence similarity 3, member D | HGNC:18665 |
| FAM46C | family with sequence similarity 46, member C | HGNC:24712 |
| FAM69C | family with sequence similarity 69, member C | HGNC:31729 |
| FAM70A | family with sequence similarity 70, member A | HGNC:26086 |
| FAM73B | family with sequence similarity 73, member B | HGNC:23621 |
| FAM75D1 | family with sequence similarity 75, member D1 | HGNC:37283 |
| FAM78A | family with sequence similarity 78, member A | HGNC:25465 |
| FAM82A2 | family with sequence similarity 82, member A2 | HGNC:25550 |
| FAM83A | family with sequence similarity 83, member A | HGNC:28210 |
| FAM84A | family with sequence similarity 84, member A | HGNC:20743 |
| FAM84B | family with sequence similarity 84, member B | HGNC:24166 |
| FAR2 | fatty acyl CoA reductase 2 | HGNC:25531 |
| FASN | fatty acid synthase | HGNC:3594 |
| FASTKD1 | FAST kinase domains 1 | HGNC:26150 |
| FBLN2 | fibulin 2 | HGNC:3601 |
| FBLN5 | fibulin 5 | HGNC:3602 |
| FBN1 | fibrillin 1 | HGNC:3603 |
| FBP1 | fructose-1,6-bisphosphatase 1 | HGNC:3606 |
| FBP2 | fructose-1,6-bisphosphatase 2 | HGNC:3607 |
| FBXO9 | F-box protein 9 | HGNC:13588 |
| FCER2 | Fc fragment of IgE, low affinity II, receptor for (CD23) | HGNC:3612 |
| FCGBP | Fc fragment of IgG binding protein | HGNC:13572 |
| FCGR2A | Fc fragment of IgG, low affinity IIa, receptor (CD32) | HGNC:3616 |
| FCGR2B | Fc fragment of IgG, low affinity IIb, receptor (CD32) | HGNC:3618 |
| FCGR3A | Fc fragment of IgG, low affinity IIIa, receptor (CD16a) | HGNC:3619 |
| FCGRT | Fc fragment of IgG, receptor, transporter, alpha | HGNC:3621 |
| FCN1 | ficolin (collagen/fibrinogen domain containing) 1 | HGNC:3623 |
| FDFT1 | farnesyl-diphosphate farnesyltransferase 1 | HGNC:3629 |
| FDPS | farnesyl diphosphate synthase | HGNC:3631 |
| FEN1 | flap structure-specific endonuclease 1 | HGNC:3650 |
| FERMT2 | fermitin family member 2 | HGNC:15767 |
| FERMT3 | fermitin family member 3 | HGNC:23151 |
| FETUB | fetuin B | HGNC:3658 |
| FGF16 | fibroblast growth factor 16 | HGNC:3672 |
| FGF18 | fibroblast growth factor 18 | HGNC:3674 |
| FGF9 | fibroblast growth factor 9 (glia-activating factor) | HGNC:3687 |
| FGFR1 | fibroblast growth factor receptor 1 | HGNC:3688 |
| FGFR2 | fibroblast growth factor receptor 2 | HGNC:3689 |
| FHL1 | four and a half LIM domains 1 | HGNC:3702 |
| FIBIN | fin bud initiation factor homolog (zebrafish) | HGNC:33747 |
| FIGF | c-fos induced growth factor (vascular endothelial growth factor D) | HGNC:3708 |
| FILIP1 | filamin A interacting protein 1 | HGNC:21015 |
| FITM2 | fat storage-inducing transmembrane protein 2 | HGNC:16135 |
| FLNA | filamin A, alpha | HGNC:3754 |
| FLNC | filamin C, gamma | HGNC:3756 |
| FLRT2 | fibronectin leucine rich transmembrane protein 2 | HGNC:3761 |
| FLT4 | fms-related tyrosine kinase 4 | HGNC:3767 |
| FMNL1 | formin-like 1 | HGNC:1212 |
| FMO2 | flavin containing monooxygenase 2 (non-functional) | HGNC:3770 |
| FMO3 | flavin containing monooxygenase 3 | HGNC:3771 |
| FMOD | fibromodulin | HGNC:3774 |
| FOS | FBJ murine osteosarcoma viral oncogene homolog | HGNC:3796 |
| FOSB | FBJ murine osteosarcoma viral oncogene homolog B | HGNC:3797 |
| FOSL1 | FOS-like antigen 1 | HGNC:13718 |
| FOXC1 | forkhead box C1 | HGNC:3800 |
| FOXQ1 | forkhead box Q1 | HGNC:20951 |
| FPR2 | formyl peptide receptor 2 | HGNC:3827 |
| FPR3 | formyl peptide receptor 3 | HGNC:3828 |
| FREM1 | FRAS1 related extracellular matrix 1 | HGNC:23399 |
| FRZB | frizzled-related protein | HGNC:3959 |
| FSTL3 | follistatin-like 3 (secreted glycoprotein) | HGNC:3973 |
| FTL | ferritin, light polypeptide | HGNC:3999 |
| FUT3 | fucosyltransferase 3 (galactoside 3(4)-L-fucosyltransferase, Lewis blood group) | HGNC:4014 |
| FXYD5 | FXYD domain containing ion transport regulator 5 | HGNC:4029 |
| FYN | FYN oncogene related to SRC, FGR, YES | HGNC:4037 |
| FZD4 | frizzled family receptor 4 | HGNC:4042 |
| FZD5 | frizzled family receptor 5 | HGNC:4043 |
| FZD7 | frizzled family receptor 7 | HGNC:4045 |
| G0S2 | G0/G1switch 2 | HGNC:30229 |
| G6PD | glucose-6-phosphate dehydrogenase | HGNC:4057 |
| GABBR2 | gamma-aminobutyric acid (GABA) B receptor, 2 | HGNC:4507 |
| GABRA4 | gamma-aminobutyric acid (GABA) A receptor, alpha 4 | HGNC:4078 |
| GABRP | gamma-aminobutyric acid (GABA) A receptor, pi | HGNC:4089 |
| GADD45G | growth arrest and DNA-damage-inducible, gamma | HGNC:4097 |
| GAGE12D | G antigen 12D | HGNC:31904 |
| GAGE12G | G antigen 12G | HGNC:31907 |
| GAGE12I | G antigen 12I | HGNC:4105 |
| GAGE2B | G antigen 2B | HGNC:31957 |
| GAGE2C | G antigen 2C | HGNC:31958 |
| GAGE2D | G antigen 2D | HGNC:31959 |
| GAL | galanin prepropeptide | HGNC:4114 |
| GALNTL1 | UDP-N-acetyl-alpha-D-galactosamine:polypeptide N- | HGNC:23233 |
| | acetylgalactosaminyltransferase-like 1 | |
| GALNTL2 | UDP-N-acetyl-alpha-D-galactosamine:polypeptide N- | HGNC:21531 |
| | acetylgalactosaminyltransferase-like 2 | |
| GALNTL5 | UDP-N-acetyl-alpha-D-galactosamine:polypeptide N- | HGNC:21725 |
| | acetylgalactosaminyltransferase-like 5 | |
| GALNTL6 | UDP-N-acetyl-alpha-D-galactosamine:polypeptide N- | HGNC:33844 |
| | acetylgalactosaminyltransferase-like 6 | |
| GAP43 | growth associated protein 43 | HGNC:4140 |
| GAS1 | growth arrest-specific 1 | HGNC:4165 |
| GATA2 | GATA binding protein 2 | HGNC:4171 |
| GATA6 | GATA binding protein 6 | HGNC:4174 |
| GBP3 | guanylate binding protein 3 | HGNC:4184 |
| GBP4 | guanylate binding protein 4 | HGNC:20480 |
| GBP6 | guanylate binding protein family, member 6 | HGNC:25395 |
| GCLM | glutamate-cysteine ligase, modifier subunit | HGNC:4312 |
| GCNT4 | glucosaminyl (N-acetyl) transferase 4, core 2 | HGNC:17973 |
| GCSH | glycine cleavage system protein H (aminomethyl carrier) | HGNC:4208 |
| GDA | guanine deaminase | HGNC:4212 |
| GDF10 | growth differentiation factor 10 | HGNC:4215 |
| GDPD2 | glycerophosphodiester phosphodiesterase domain containing 2 | HGNC:25974 |
| GFM2 | G elongation factor, mitochondrial 2 | HGNC:29682 |
| GFRA1 | GDNF family receptor alpha 1 | HGNC:4243 |
| GGPS1 | geranylgeranyl diphosphate synthase 1 | HGNC:4249 |
| GGT6 | gamma-glutamyltransferase 6 | HGNC:26891 |
| GIMAP4 | GTPase, IMAP family member 4 | HGNC:21872 |
| GJA3 | gap junction protein, alpha 3, 46 kDa | HGNC:4277 |
| GJB2 | gap junction protein, beta 2, 26 kDa | HGNC:4284 |
| GJB6 | gap junction protein, beta 6, 30 kDa | HGNC:4288 |
| GJC2 | gap junction protein, gamma 2, 47 kDa | HGNC:17494 |
| GK | glycerol kinase | HGNC:4289 |
| GK5 | glycerol kinase 5 (putative) | HGNC:28635 |
| GLDC | glycine dehydrogenase (decarboxylating) | HGNC:4313 |
| GLIPR1 | GLI pathogenesis-related 1 | HGNC:17001 |
| GLRX | glutaredoxin (thioltransferase) | HGNC:4330 |
| GLRX5 | glutaredoxin 5 | HGNC:20134 |
| GLYAT | glycine-N-acyltransferase | HGNC:13734 |
| GLYATL1 | glycine-N-acyltransferase-like 1 | HGNC:30519 |
| GLYATL1P4 | glycine-N-acyltransferase-like 1 pseudogene 4 | HGNC:37864 |
| GNLY | granulysin | HGNC:4414 |
| GNPAT | glyceronephosphate O-acyltransferase | HGNC:4416 |
| GOT1 | glutamic-oxaloacetic transaminase 1, soluble (aspartate aminotransferase 1) | HGNC:4432 |
| GPAM | glycerol-3-phosphate acyltransferase, mitochondrial | HGNC:24865 |
| GPBAR1 | G protein-coupled bile acid receptor 1 | HGNC:19680 |
| GPC3 | glypican 3 | HGNC:4451 |
| GPER | G protein-coupled estrogen receptor 1 | HGNC:4485 |
| GPIHBP1 | glycosylphosphatidylinositol anchored high density lipoprotein binding protein 1 | HGNC:24945 |
| GPR124 | G protein-coupled receptor 124 | HGNC:17849 |
| GPR133 | G protein-coupled receptor 133 | HGNC:19893 |
| GPR143 | G protein-coupled receptor 143 | HGNC:20145 |
| GPR15 | G protein-coupled receptor 15 | HGNC:4469 |
| GPR174 | G protein-coupled receptor 174 | HGNC:30245 |
| GPR18 | G protein-coupled receptor 18 | HGNC:4472 |
| GPR182 | G protein-coupled receptor 182 | HGNC:13708 |
| GPR64 | G protein-coupled receptor 64 | HGNC:4516 |
| GPR98 | G protein-coupled receptor 98 | HGNC:17416 |
| GPRC5D | G protein-coupled receptor, family C, group 5, member D | HGNC:13310 |
| GPRIN2 | G protein regulated inducer of neurite outgrowth 2 | HGNC:23730 |
| GPSM3 | G-protein signaling modulator 3 | HGNC:13945 |
| GPT | glutamic-pyruvate transaminase (alanine aminotransferase) | HGNC:4552 |
| GPT2 | glutamic pyruvate transaminase (alanine aminotransferase) 2 | HGNC:18062 |
| GRAP2 | GRB2-related adaptor protein 2 | HGNC:4563 |
| GREB1 | growth regulation by estrogen in breast cancer 1 | HGNC:24885 |
| GREM1 | gremlin 1 | HGNC:2001 |
| GRIN2A | glutamate receptor, ionotropic, N-methyl D-aspartate 2A | HGNC:4585 |
| GRIN3A | glutamate receptor, ionotropic, N-methyl-D-aspartate 3A | HGNC:16767 |
| GRK5 | G protein-coupled receptor kinase 5 | HGNC:4544 |
| GRM3 | glutamate receptor, metabotropic 3 | HGNC:4595 |
| GRM7 | glutamate receptor, metabotropic 7 | HGNC:4599 |
| GRP | gastrin-releasing peptide | HGNC:4605 |
| GSDMA | gasdermin A | HGNC:13311 |
| GTSF1 | gametocyte specific factor 1 | HGNC:26565 |
| GUCY2C | guanylate cyclase 2C (heat stable enterotoxin receptor) | HGNC:4688 |
| GYG2 | glycogenin 2 | HGNC:4700 |
| GYPC | glycophorin C (Gerbich blood group) | HGNC:4704 |
| GYS2 | glycogen synthase 2 (liver) | HGNC:4707 |
| GZMA | granzyme A (granzyme 1, cytotoxic T-lymphocyte-associated serine esterase 3) | HGNC:4708 |
| GZMB | granzyme B (granzyme 2, cytotoxic T-lymphocyte-associated serine esterase 1) | HGNC:4709 |
| GZMH | granzyme H (cathepsin G-like 2, protein h-CCPX) | HGNC:4710 |
| GZMK | granzyme K (granzyme 3; tryptase II) | HGNC:4711 |
| GZMM | granzyme M (lymphocyte met-ase 1) | HGNC:4712 |
| HACL1 | 2-hydroxyacyl-CoA lyase 1 | HGNC:17856 |
| HADH | hydroxyacyl-CoA dehydrogenase | HGNC:4799 |
| HAO2 | hydroxyacid oxidase 2 (long chain) | HGNC:4810 |
| HAPLN1 | hyaluronan and proteoglycan link protein 1 | HGNC:2380 |
| HBA1 | hemoglobin, alpha 1 | HGNC:4823 |
| HBA2 | hemoglobin, alpha 2 | HGNC:4824 |
| HBB | hemoglobin, beta | HGNC:4827 |
| HBD | hemoglobin, delta | HGNC:4829 |
| HBEGF | heparin-binding EGF-like growth factor | HGNC:3059 |
| HBG2 | hemoglobin, gamma G | HGNC:4832 |
| HCLS1 | hematopoietic cell-specific Lyn substrate 1 | HGNC:4844 |
| HEATR4 | HEAT repeat containing 4 | HGNC:16761 |
| HEPACAM | hepatic and glial cell adhesion molecule | HGNC:26361 |
| HEPACAM2 | HEPACAM family member 2 | HGNC:27364 |
| HEPHL1 | hephaestin-like 1 | HGNC:30477 |
| HEPN1 | hepatocellular carcinoma, down-regulated 1 | HGNC:34400 |
| HGD | homogentisate 1,2-dioxygenase | HGNC:4892 |
| HHATL | hedgehog acyltransferase-like | HGNC:13242 |
| HIBCH | 3-hydroxyisobutyryl-CoA hydrolase | HGNC:4908 |
| HIC1 | hypermethylated in cancer 1 | HGNC:4909 |
| HINT1 | histidine triad nucleotide binding protein 1 | HGNC:4912 |
| HIP1 | huntingtin interacting protein 1 | HGNC:4913 |
| HIST1H1C | histone cluster 1, H1c | HGNC:4716 |
| HIST1H1D | histone cluster 1, H1d | HGNC:4717 |
| HIST1H2AE | histone cluster 1, H2ae | HGNC:4724 |
| HIST1H2BC | histone cluster 1, H2bc | HGNC:4757 |
| HIST1H2BK | histone cluster 1, H2bk | HGNC:13954 |
| HIST3H2A | histone cluster 3, H2a | HGNC:20507 |
| HK2 | hexokinase 2 | HGNC:4923 |
| HLA-A | major histocompatibility complex, class I, A | HGNC:4931 |
| HLA-B | major histocompatibility complex, class I, B | HGNC:4932 |
| HLA-C | major histocompatibility complex, class I, C | HGNC:4933 |
| HLA-DMB | major histocompatibility complex, class II, DM beta | HGNC:4935 |
| HLA-DOA | major histocompatibility complex, class II, DO alpha | HGNC:4936 |
| HLA-DPA1 | major histocompatibility complex, class II, DP alpha 1 | HGNC:4938 |
| HLA-DQA1 | major histocompatibility complex, class II, DQ alpha 1 | HGNC:4942 |
| HLA-DQA2 | major histocompatibility complex, class II, DQ alpha 2 | HGNC:4943 |
| HLA-DRA | major histocompatibility complex, class II, DR alpha | HGNC:4947 |
| HLA-DRB1 | major histocompatibility complex, class II, DR beta 1 | HGNC:4948 |
| HLA-DRB5 | major histocompatibility complex, class II, DR beta 5 | HGNC:4953 |
| HLA-E | major histocompatibility complex, class I, E | HGNC:4962 |
| HLA-F | major histocompatibility complex, class I, F | HGNC:4963 |
| HLCS | holocarboxylase synthetase (biotin-(proprionyl-CoA-carboxylase (ATP- | HGNC:4976 |
| | hydrolysing)) ligase) | |
| HMCN2 | hemicentin 2 | HGNC:21293 |
| HMGB3 | high mobility group box 3 | HGNC:5004 |
| HMGCL | 3-hydroxymethyl-3-methylglutaryl-CoA lyase | HGNC:5005 |
| HMGCR | 3-hydroxy-3-methylglutaryl-CoA reductase | HGNC:5006 |
| HMGCS1 | 3-hydroxy-3-methylglutaryl-CoA synthase 1 (soluble) | HGNC:5007 |
| HOMER1 | homer homolog 1 (Drosophila) | HGNC:17512 |
| HPGDS | hematopoietic prostaglandin D synthase | HGNC:17890 |
| HPSE2 | heparanase 2 | HGNC:18374 |
| HRASLS5 | HRAS-like suppressor family, member 5 | HGNC:24978 |
| HRSP12 | heat-responsive protein 12 | HGNC:16897 |
| HSD11B1 | hydroxysteroid (11-beta) dehydrogenase 1 | HGNC:5208 |
| HSD11B2 | hydroxysteroid (11-beta) dehydrogenase 2 | HGNC:5209 |
| HSD17B11 | hydroxysteroid (17-beta) dehydrogenase 11 | HGNC:22960 |
| HSD17B13 | hydroxysteroid (17-beta) dehydrogenase 13 | HGNC:18685 |
| HSD17B2 | hydroxysteroid (17-beta) dehydrogenase 2 | HGNC:5211 |
| HSD3B1 | hydroxy-delta-5-steroid dehydrogenase, 3 beta- and steroid delta-isomerase 1 | HGNC:5217 |
| HSD3B2 | hydroxy-delta-5-steroid dehydrogenase, 3 beta- and steroid delta-isomerase 2 | HGNC:5218 |
| HSPA12A | heat shock 70 kDa protein 12A | HGNC:19022 |
| HSPB6 | heat shock protein, alpha-crystallin-related, B6 | HGNC:26511 |
| HSPB7 | heat shock 27 kDa protein family, member 7 (cardiovascular) | HGNC:5249 |
| HSPE1 | heat shock 10 kDa protein 1 (chaperonin 10) | HGNC:5269 |
| HSPG2 | heparan sulfate proteoglycan 2 | HGNC:5273 |
| HTRA3 | HtrA serine peptidase 3 | HGNC:30406 |
| HYAL1 | hyaluronoglucosaminidase 1 | HGNC:5320 |
| ID2 | inhibitor of DNA binding 2, dominant negative helix-loop-helix protein | HGNC:5361 |
| ID3 | inhibitor of DNA binding 3, dominant negative helix-loop-helix protein | HGNC:5362 |
| IDH1 | isocitrate dehydrogenase 1 (NADP+), soluble | HGNC:5382 |
| IDH2 | isocitrate dehydrogenase 2 (NADP+), mitochondrial | HGNC:5383 |
| IDI1 | isopentenyl-diphosphate delta isomerase 1 | HGNC:5387 |
| IER3 | immediate early response 3 | HGNC:5392 |
| IFI27 | interferon, alpha-inducible protein 27 | HGNC:5397 |
| IFI44L | interferon-induced protein 44-like | HGNC:17817 |
| IFIT2 | interferon-induced protein with tetratricopeptide repeats 2 | HGNC:5409 |
| IFIT3 | interferon-induced protein with tetratricopeptide repeats 3 | HGNC:5411 |
| IFITM1 | interferon induced transmembrane protein 1 | HGNC:5412 |
| IFITM3 | interferon induced transmembrane protein 3 | HGNC:5414 |
| IFNG | interferon, gamma | HGNC:5438 |
| IFRD1 | interferon-related developmental regulator 1 | HGNC:5456 |
| IGF1 | insulin-like growth factor 1 (somatomedin C) | HGNC:5464 |
| IGF2 | insulin-like growth factor 2 (somatomedin A) | HGNC:5466 |
| IGFBP3 | insulin-like growth factor binding protein 3 | HGNC:5472 |
| IGFL2 | IGF-like family member 2 | HGNC:32929 |
| IGFL4 | IGF-like family member 4 | HGNC:32931 |
| IGJ | immunoglobulin J polypeptide, linker protein for immunoglobulin alpha and mu | HGNC:5713 |
| | polypeptides | |
| IGLL5 | immunoglobulin lambda-like polypeptide 5 | HGNC:38476 |
| IGLON5 | IgLON family member 5 | HGNC:34550 |
| IGSF10 | immunoglobulin superfamily, member 10 | HGNC:26384 |
| IGSF6 | immunoglobulin superfamily, member 6 | HGNC:5953 |
| IKZF1 | IKAROS family zinc finger 1 (Ikaros) | HGNC:13176 |
| IKZF3 | IKAROS family zinc finger 3 (Aiolos) | HGNC:13178 |
| IL10RA | interleukin 10 receptor, alpha | HGNC:5964 |
| IL15RA | interleukin 15 receptor, alpha | HGNC:5978 |
| IL17D | interleukin 17D | HGNC:5984 |
| IL1B | interleukin 1, beta | HGNC:5992 |
| IL2RG | interleukin 2 receptor, gamma | HGNC:6010 |
| IL32 | interleukin 32 | HGNC:16830 |
| IL33 | interleukin 33 | HGNC:16028 |
| IL7R | interleukin 7 receptor | HGNC:6024 |
| IL8 | interleukin 8 | HGNC:6025 |
| ILDR1 | immunoglobulin-like domain containing receptor 1 | HGNC:28741 |
| INHBB | inhibin, beta B | HGNC:6067 |
| INMT | indolethylamine N-methyltransferase | HGNC:6069 |
| INPPL1 | inositol polyphosphate phosphatase-like 1 | HGNC:6080 |
| INSIG1 | insulin induced gene 1 | HGNC:6083 |
| INSM1 | insulinoma-associated 1 | HGNC:6090 |
| IQCJ | IQ motif containing J | HGNC:32406 |
| IQSEC3 | IQ motif and Sec7 domain 3 | HGNC:29193 |
| IRF1 | interferon regulatory factor 1 | HGNC:6116 |
| IRF8 | interferon regulatory factor 8 | HGNC:5358 |
| IRGM | immunity-related GTPase family, M | HGNC:29597 |
| IRX6 | iroquois homeobox 6 | HGNC:14675 |
| ISLR | immunoglobulin superfamily containing leucine-rich repeat | HGNC:6133 |
| ISM1 | isthmin 1 homolog (zebrafish) | HGNC:16213 |
| ISOC1 | isochorismatase domain containing 1 | HGNC:24254 |
| ITGA1 | integrin, alpha 1 | HGNC:6134 |
| ITGA2 | integrin, alpha 2 (CD49B, alpha 2 subunit of VLA-2 receptor) | HGNC:6137 |
| ITGA7 | integrin, alpha 7 | HGNC:6143 |
| ITGA8 | integrin, alpha 8 | HGNC:6144 |
| ITGAL | integrin, alpha L (antigen CD11A (p180), lymphocyte function-associated | HGNC:6148 |
| | antigen 1; alpha polypeptide) | |
| ITGAM | integrin, alpha M (complement component 3 receptor 3 subunit) | HGNC:6149 |
| ITGB1BP1 | integrin beta 1 binding protein 1 | HGNC:23927 |
| ITGB2 | integrin, beta 2 (complement component 3 receptor 3 and 4 subunit) | HGNC:6155 |
| ITIH5 | inter-alpha-trypsin inhibitor heavy chain family, member 5 | HGNC:21449 |
| ITK | IL2-inducible T-cell kinase | HGNC:6171 |
| ITSN1 | intersectin 1 (SH3 domain protein) | HGNC:6183 |
| IVD | isovaleryl-CoA dehydrogenase | HGNC:6186 |
| IYD | iodotyrosine deiodinase | HGNC:21071 |
| JAM3 | junctional adhesion molecule 3 | HGNC:15532 |
| JPH2 | junctophilin 2 | HGNC:14202 |
| KANK2 | KN motif and ankyrin repeat domains 2 | HGNC:29300 |
| KCNA3 | potassium voltage-gated channel, shaker-related subfamily, member 3 | HGNC:6221 |
| KCNA6 | potassium voltage-gated channel, shaker-related subfamily, member 6 | HGNC:6225 |
| KCNB1 | potassium voltage-gated channel, Shab-related subfamily, member 1 | HGNC:6231 |
| KCNH5 | potassium voltage-gated channel, subfamily H (eag-related), member 5 | HGNC:6254 |
| KCNIP2 | Kv channel interacting protein 2 | HGNC:15522 |
| KCNIP3 | Kv channel interacting protein 3, calsenilin | HGNC:15523 |
| KCNJ13 | potassium inwardly-rectifying channel, subfamily J, member 13 | HGNC:6259 |
| KCNJ15 | potassium inwardly-rectifying channel, subfamily J, member 15 | HGNC:6261 |
| KCNK2 | potassium channel, subfamily K, member 2 | HGNC:6277 |
| KCNMA1 | potassium large conductance calcium-activated channel, subfamily M, alpha | HGNC:6284 |
| | member 1 | |
| KCNMB1 | potassium large conductance calcium-activated channel, subfamily M, beta | HGNC:6285 |
| | member 1 | |
| KDELC2 | KDEL (Lys-Asp-Glu-Leu) containing 2 | HGNC:28496 |
| KIAA0513 | KIAA0513 | HGNC:29058 |
| KIAA0754 | KIAA0754 | HGNC:29111 |
| KIAA1191 | KIAA1191 | HGNC:29209 |
| KIF1A | kinesin family member 1A | HGNC:888 |
| KIF3C | kinesin family member 3C | HGNC:6321 |
| KIRREL | kin of IRRE like (Drosophila) | HGNC:15734 |
| KLHDC8B | kelch domain containing 8B | HGNC:28557 |
| KLHL31 | kelch-like 31 (Drosophila) | HGNC:21353 |
| KLK10 | kallikrein-related peptidase 10 | HGNC:6358 |
| KLK12 | kallikrein-related peptidase 12 | HGNC:6360 |
| KLK13 | kallikrein-related peptidase 13 | HGNC:6361 |
| KLK6 | kallikrein-related peptidase 6 | HGNC:6367 |
| KLRF1 | killer cell lectin-like receptor subfamily F, member 1 | HGNC:13342 |
| KPRP | keratinocyte proline-rich protein | HGNC:31823 |
| KREMEN2 | kringle containing transmembrane protein 2 | HGNC:18797 |
| KRT16 | keratin 16 | HGNC:6423 |
| KRT17 | keratin 17 | HGNC:6427 |
| KRT20 | keratin 20 | HGNC:20412 |
| KRT25 | keratin 25 | HGNC:30839 |
| KRT26 | keratin 26 | HGNC:30840 |
| KRT27 | keratin 27 | HGNC:30841 |
| KRT28 | keratin 28 | HGNC:30842 |
| KRT31 | keratin 31 | HGNC:6448 |
| KRT32 | keratin 32 | HGNC:6449 |
| KRT33A | keratin 33A | HGNC:6450 |
| KRT33B | keratin 33B | HGNC:6451 |
| KRT34 | keratin 34 | HGNC:6452 |
| KRT35 | keratin 35 | HGNC:6453 |
| KRT36 | keratin 36 | HGNC:6454 |
| KRT38 | keratin 38 | HGNC:6456 |
| KRT39 | keratin 39 | HGNC:32971 |
| KRT4 | keratin 4 | HGNC:6441 |
| KRT40 | keratin 40 | HGNC:26707 |
| KRT6A | keratin 6A | HGNC:6443 |
| KRT6B | keratin 6B | HGNC:6444 |
| KRT6C | keratin 6C | HGNC:20406 |
| KRT71 | keratin 71 | HGNC:28927 |
| KRT72 | keratin 72 | HGNC:28932 |
| KRT73 | keratin 73 | HGNC:28928 |
| KRT74 | keratin 74 | HGNC:28929 |
| KRT75 | keratin 75 | HGNC:24431 |
| KRT79 | keratin 79 | HGNC:28930 |
| KRT81 | keratin 81 | HGNC:6458 |
| KRT82 | keratin 82 | HGNC:6459 |
| KRT83 | keratin 83 | HGNC:6460 |
| KRT84 | keratin 84 | HGNC:6461 |
| KRT85 | keratin 85 | HGNC:6462 |
| KRT86 | keratin 86 | HGNC:6463 |
| KRT9 | keratin 9 | HGNC:6447 |
| KRTAP1-1 | keratin associated protein 1-1 | HGNC:16772 |
| KRTAP1-3 | keratin associated protein 1-3 | HGNC:16771 |
| KRTAP1-5 | keratin associated protein 1-5 | HGNC:16777 |
| KRTAP10-1 | keratin associated protein 10-1 | HGNC:22966 |
| KRTAP10-10 | keratin associated protein 10-10 | HGNC:22972 |
| KRTAP10-11 | keratin associated protein 10-11 | HGNC:20528 |
| KRTAP10-12 | keratin associated protein 10-12 | HGNC:20533 |
| KRTAP10-2 | keratin associated protein 10-2 | HGNC:22967 |
| KRTAP10-3 | keratin associated protein 10-3 | HGNC:22968 |
| KRTAP10-4 | keratin associated protein 10-4 | HGNC:20521 |
| KRTAP10-5 | keratin associated protein 10-5 | HGNC:22969 |
| KRTAP10-6 | keratin associated protein 10-6 | HGNC:20523 |
| KRTAP10-7 | keratin associated protein 10-7 | HGNC:22970 |
| KRTAP10-8 | keratin associated protein 10-8 | HGNC:20525 |
| KRTAP10-9 | keratin associated protein 10-9 | HGNC:22971 |
| KRTAP11-1 | keratin associated protein 11-1 | HGNC:18922 |
| KRTAP12-1 | keratin associated protein 12-1 | HGNC:20529 |
| KRTAP12-2 | keratin associated protein 12-2 | HGNC:20530 |
| KRTAP12-3 | keratin associated protein 12-3 | HGNC:20531 |
| KRTAP13-1 | keratin associated protein 13-1 | HGNC:18924 |
| KRTAP13-2 | keratin associated protein 13-2 | HGNC:18923 |
| KRTAP17-1 | keratin associated protein 17-1 | HGNC:18917 |
| KRTAP19-1 | keratin associated protein 19-1 | HGNC:18936 |
| KRTAP19-3 | keratin associated protein 19-3 | HGNC:18938 |
| KRTAP19-5 | keratin associated protein 19-5 | HGNC:18940 |
| KRTAP19-7 | keratin associated protein 19-7 | HGNC:18942 |
| KRTAP2-1 | keratin associated protein 2-1 | HGNC:16775 |
| KRTAP2-2 | keratin associated protein 2-2 | HGNC:18905 |
| KRTAP2-4 | keratin associated protein 2-4 | HGNC:18891 |
| KRTAP20-1 | keratin associated protein 20-1 | HGNC:18943 |
| KRTAP20-2 | keratin associated protein 20-2 | HGNC:18944 |
| KRTAP21-2 | keratin associated protein 21-2 | HGNC:18946 |
| KRTAP24-1 | keratin associated protein 24-1 | HGNC:33902 |
| KRTAP26-1 | keratin associated protein 26-1 | HGNC:33760 |
| KRTAP3-1 | keratin associated protein 3-1 | HGNC:16778 |
| KRTAP3-2 | keratin associated protein 3-2 | HGNC:16779 |
| KRTAP3-3 | keratin associated protein 3-3 | HGNC:18890 |
| KRTAP4-1 | keratin associated protein 4-1 | HGNC:18907 |
| KRTAP4-11 | keratin associated protein 4-11 | HGNC:18911 |
| KRTAP4-12 | keratin associated protein 4-12 | HGNC:16776 |
| KRTAP4-2 | keratin associated protein 4-2 | HGNC:18900 |
| KRTAP4-3 | keratin associated protein 4-3 | HGNC:18908 |
| KRTAP4-4 | keratin associated protein 4-4 | HGNC:16928 |
| KRTAP4-5 | keratin associated protein 4-5 | HGNC:18899 |
| KRTAP4-6 | keratin associated protein 4-6 | HGNC:18909 |
| KRTAP4-7 | keratin associated protein 4-7 | HGNC:18898 |
| KRTAP4-8 | keratin associated protein 4-8 | HGNC:17230 |
| KRTAP4-9 | keratin associated protein 4-9 | HGNC:18910 |
| KRTAP5-1 | keratin associated protein 5-1 | HGNC:23596 |
| KRTAP5-10 | keratin associated protein 5-10 | HGNC:23605 |
| KRTAP5-11 | keratin associated protein 5-11 | HGNC:23606 |
| KRTAP5-2 | keratin associated protein 5-2 | HGNC:23597 |
| KRTAP5-3 | keratin associated protein 5-3 | HGNC:23598 |
| KRTAP5-4 | keratin associated protein 5-4 | HGNC:23599 |
| KRTAP5-5 | keratin associated protein 5-5 | HGNC:23601 |
| KRTAP5-6 | keratin associated protein 5-6 | HGNC:23600 |
| KRTAP5-7 | keratin associated protein 5-7 | HGNC:23602 |
| KRTAP5-8 | keratin associated protein 5-8 | HGNC:23603 |
| KRTAP5-9 | keratin associated protein 5-9 | HGNC:23604 |
| KRTAP7-1 | keratin associated protein 7-1 (gene/pseudogene) | HGNC:18934 |
| KRTAP8-1 | keratin associated protein 8-1 | HGNC:18935 |
| KRTAP9-2 | keratin associated protein 9-2 | HGNC:16926 |
| KRTAP9-3 | keratin associated protein 9-3 | HGNC:16927 |
| KRTAP9-4 | keratin associated protein 9-4 | HGNC:18902 |
| KRTAP9-8 | keratin associated protein 9-8 | HGNC:17231 |
| KRTAP9-9 | keratin associated protein 9-9 | HGNC:16773 |
| LAIR1 | leukocyte-associated immunoglobulin-like receptor 1 | HGNC:6477 |
| LAMA2 | laminin, alpha 2 | HGNC:6482 |
| LAMA4 | laminin, alpha 4 | HGNC:6484 |
| LAMB4 | laminin, beta 4 | HGNC:6491 |
| LAMC1 | laminin, gamma 1 (formerly LAMB2) | HGNC:6492 |
| LAPTM5 | lysosomal protein transmembrane 5 | HGNC:29612 |
| LAT | linker for activation of T cells | HGNC:18874 |
| LAX1 | lymphocyte transmembrane adaptor 1 | HGNC:26005 |
| LBP | lipopolysaccharide binding protein | HGNC:6517 |
| LCE1A | late cornified envelope 1A | HGNC:29459 |
| LCE2A | late cornified envelope 2A | HGNC:29469 |
| LCE2B | late cornified envelope 2B | HGNC:16610 |
| LCE2C | late cornified envelope 2C | HGNC:29460 |
| LCE5A | late cornified envelope 5A | HGNC:16614 |
| LCK | lymphocyte-specific protein tyrosine kinase | HGNC:6524 |
| LCN2 | lipocalin 2 | HGNC:6526 |
| LCP1 | lymphocyte cytosolic protein 1 (L-plastin) | HGNC:6528 |
| LCP2 | lymphocyte cytosolic protein 2 (SH2 domain containing leukocyte | HGNC:6529 |
| | protein of 76 kDa) | |
| LCT | lactase | HGNC:6530 |
| LDHB | lactate dehydrogenase B | HGNC:6541 |
| LDHD | lactate dehydrogenase D | HGNC:19708 |
| LDLR | low density lipoprotein receptor | HGNC:6547 |
| LEP | leptin | HGNC:6553 |
| LEPR | leptin receptor | HGNC:6554 |
| LETM1 | leucine zipper-EF-hand containing transmembrane protein 1 | HGNC:6556 |
| LGALS9 | lectin, galactoside-binding, soluble, 9 | HGNC:6570 |
| LGI3 | leucine-rich repeat LGI family, member 3 | HGNC:18711 |
| LGR5 | leucine-rich repeat containing G protein-coupled receptor 5 | HGNC:4504 |
| LHFP | lipoma HMGIC fusion partner | HGNC:6586 |
| LHFPL4 | lipoma HMGIC fusion partner-like 4 | HGNC:29568 |
| LHX2 | LIM homeobox 2 | HGNC:6594 |
| LHX3 | LIM homeobox 3 | HGNC:6595 |
| LILRB5 | leukocyte immunoglobulin-like receptor, subfamily B (with TM and | HGNC:6609 |
| | ITIM domains), member 5 | |
| LIMCH1 | LIM and calponin homology domains 1 | HGNC:29191 |
| LIPE | lipase, hormone-sensitive | HGNC:6621 |
| LIPG | lipase, endothelial | HGNC:6623 |
| LIPH | lipase, member H | HGNC:18483 |
| LIPK | lipase, family member K | HGNC:23444 |
| LMO3 | LIM domain only 3 (rhombotin-like 2) | HGNC:6643 |
| LMOD1 | leiomodin 1 (smooth muscle) | HGNC:6647 |
| LOX | lysyl oxidase | HGNC:6664 |
| LPCAT3 | lysophosphatidylcholine acyltransferase 3 | HGNC:30244 |
| LPHN3 | latrophilin 3 | HGNC:20974 |
| LPIN1 | lipin 1 | HGNC:13345 |
| LPL | lipoprotein lipase | HGNC:6677 |
| LPO | lactoperoxidase | HGNC:6678 |
| LRG1 | leucine-rich alpha-2-glycoprotein 1 | HGNC:29480 |
| LRIT2 | leucine-rich repeat, immunoglobulin-like and transmembrane domains 2 | HGNC:23443 |
| LRP1 | low density lipoprotein receptor-related protein 1 | HGNC:6692 |
| LRRC10B | leucine rich repeat containing 10B | HGNC:37215 |
| LRRC31 | leucine rich repeat containing 31 | HGNC:26261 |
| LRRC32 | leucine rich repeat containing 32 | HGNC:4161 |
| LRRN4CL | LRRN4 C-terminal like | HGNC:33724 |
| LTB | lymphotoxin beta (TNF superfamily, member 3) | HGNC:6711 |
| LTBP2 | latent transforming growth factor beta binding protein 2 | HGNC:6715 |
| LTBP3 | latent transforming growth factor beta binding protein 3 | HGNC:6716 |
| LTF | lactotransferrin | HGNC:6720 |
| LUM | lumican | HGNC:6724 |
| LY6E | lymphocyte antigen 6 complex, locus E | HGNC:6727 |
| LY6G6D | lymphocyte antigen 6 complex, locus G6D | HGNC:13935 |
| LY9 | lymphocyte antigen 9 | HGNC:6730 |
| LYG2 | lysozyme G-like 2 | HGNC:29615 |
| LYRM5 | LYR motif containing 5 | HGNC:27052 |
| LYZ | lysozyme | HGNC:6740 |
| LZTS1 | leucine zipper, putative tumor suppressor 1 | HGNC:13861 |
| MAF | v-maf musculoaponeurotic fibrosarcoma oncogene homolog (avian) | HGNC:6776 |
| MAGEA11 | melanoma antigen family A, 11 | HGNC:6798 |
| MAGEB2 | melanoma antigen family B, 2 | HGNC:6809 |
| MAN1A1 | mannosidase, alpha, class 1A, member 1 | HGNC:6821 |
| MAN2B2 | mannosidase, alpha, class 2B, member 2 | HGNC:29623 |
| MANEAL | mannosidase, endo-alpha-like | HGNC:26452 |
| MAOB | monoamine oxidase B | HGNC:6834 |
| MAP1B | microtubule-associated protein 1B | HGNC:6836 |
| MAPK8IP2 | mitogen-activated protein kinase 8 interacting protein 2 | HGNC:6883 |
| MARCKSL1 | MARCKS-like 1 | HGNC:7142 |
| MARCO | macrophage receptor with collagenous structure | HGNC:6895 |
| MAT1A | methionine adenosyltransferase I, alpha | HGNC:6903 |
| MBOAT2 | membrane bound O-acyltransferase domain containing 2 | HGNC:25193 |
| MC5R | melanocortin 5 receptor | HGNC:6933 |
| MCAM | melanoma cell adhesion molecule | HGNC:6934 |
| MCCC1 | methylcrotonoyl-CoA carboxylase 1 (alpha) | HGNC:6936 |
| MCCC2 | methylcrotonoyl-CoA carboxylase 2 (beta) | HGNC:6937 |
| ME1 | malic enzyme 1, NADP(+)-dependent, cytosolic | HGNC:6983 |
| MECR | mitochondrial trans-2-enoyl-CoA reductase | HGNC:19691 |
| MEF2A | myocyte enhancer factor 2A | HGNC:6993 |
| MEGF11 | multiple EGF-like-domains 11 | HGNC:29635 |
| MERTK | c-mer proto-oncogene tyrosine kinase | HGNC:7027 |
| METTL7B | methyltransferase like 7B | HGNC:28276 |
| MFAP3L | microfibrillar-associated protein 3-like | HGNC:29083 |
| MFAP4 | microfibrillar-associated protein 4 | HGNC:7035 |
| MFAP5 | microfibrillar associated protein 5 | HGNC:29673 |
| MFGE8 | milk fat globule-EGF factor 8 protein | HGNC:7036 |
| MGLL | monoglyceride lipase | HGNC:17038 |
| MGP | matrix Gla protein | HGNC:7060 |
| MGST1 | microsomal glutathione S-transferase 1 | HGNC:7061 |
| MKX | mohawk homeobox | HGNC:23729 |
| MLANA | melan-A | HGNC:7124 |
| MMD | monocyte to macrophage differentiation-associated | HGNC:7153 |
| MME | membrane metallo-endopeptidase | HGNC:7154 |
| MMP28 | matrix metallopeptidase 28 | HGNC:14366 |
| MMP7 | matrix metallopeptidase 7 (matrilysin, uterine) | HGNC:7174 |
| MMP9 | matrix metallopeptidase 9 (gelatinase B, 92 kDa gelatinase, | HGNC:7176 |
| | 92 kDa type IV collagenase) | |
| MMRN1 | multimerin 1 | HGNC:7178 |
| MNDA | myeloid cell nuclear differentiation antigen | HGNC:7183 |
| MOCOS | molybdenum cofactor sulfurase | HGNC:18234 |
| MOGAT1 | monoacylglycerol O-acyltransferase 1 | HGNC:18210 |
| MOGAT2 | monoacylglycerol O-acyltransferase 2 | HGNC:23248 |
| MORN2 | MORN repeat containing 2 | HGNC:30166 |
| MPEG1 | macrophage expressed 1 | HGNC:29619 |
| MRC1 | mannose receptor, C type 1 | HGNC:7228 |
| MRPL13 | mitochondrial ribosomal protein L13 | HGNC:14278 |
| MRPL14 | mitochondrial ribosomal protein L14 | HGNC:14279 |
| MRPS25 | mitochondrial ribosomal protein S25 | HGNC:14511 |
| MRVI1 | murine retrovirus integration site 1 homolog | HGNC:7237 |
| MS4A1 | membrane-spanning 4-domains, subfamily A, member 1 | HGNC:7315 |
| MS4A18 | membrane-spanning 4-domains, subfamily A, member 18 | HGNC:37636 |
| MS4A4A | membrane-spanning 4-domains, subfamily A, member 4A | HGNC:13371 |
| MS4A6A | membrane-spanning 4-domains, subfamily A, member 6A | HGNC:13375 |
| MS4A7 | membrane-spanning 4-domains, subfamily A, member 7 | HGNC:13378 |
| MSMB | microseminoprotein, beta- | HGNC:7372 |
| MSR1 | macrophage scavenger receptor 1 | HGNC:7376 |
| MTCH2 | mitochondrial carrier 2 | HGNC:17587 |
| MTOR | mechanistic target of rapamycin (serine/threonine kinase) | HGNC:3942 |
| MUC1 | mucin 1, cell surface associated | HGNC:7508 |
| MUC15 | mucin 15, cell surface associated | HGNC:14956 |
| MUC21 | mucin 21, cell surface associated | HGNC:21661 |
| MUC7 | mucin 7, secreted | HGNC:7518 |
| MUCL1 | mucin-like 1 | HGNC:30588 |
| MUM1L1 | melanoma associated antigen (mutated) 1-like 1 | HGNC:26583 |
| MVD | mevalonate (diphospho) decarboxylase | HGNC:7529 |
| MVK | mevalonate kinase | HGNC:7530 |
| MX1 | myxovirus (influenza virus) resistance 1, interferon-inducible | HGNC:7532 |
| | protein p78 (mouse) | |
| MX2 | myxovirus (influenza virus) resistance 2 (mouse) | HGNC:7533 |
| MXRA7 | matrix-remodelling associated 7 | HGNC:7541 |
| MXRA8 | matrix-remodelling associated 8 | HGNC:7542 |
| MYBPC1 | myosin binding protein C, slow type | HGNC:7549 |
| MYEOV | myeloma overexpressed (in a subset of t(11; 14) positive | HGNC:7563 |
| | multiple myelomas) | |
| MYH11 | myosin, heavy chain 11, smooth muscle | HGNC:7569 |
| MYH3 | myosin, heavy chain 3, skeletal muscle, embryonic | HGNC:7573 |
| MYL4 | myosin, light chain 4, alkali; atrial, embryonic | HGNC:7585 |
| MYL9 | myosin, light chain 9, regulatory | HGNC:15754 |
| MYO1F | myosin IF | HGNC:7600 |
| MYO1G | myosin IG | HGNC:13880 |
| MYOCD | myocardin | HGNC:16067 |
| MYOM2 | myomesin (M-protein) 2, 165 kDa | HGNC:7614 |
| MYOZ2 | myozenin 2 | HGNC:1330 |
| NAALAD2 | N-acetylated alpha-linked acidic dipeptidase 2 | HGNC:14526 |
| NADSYN1 | NAD synthetase 1 | HGNC:29832 |
| NAMPT | nicotinamide phosphoribosyltransferase | HGNC:30092 |
| NCKAP1L | NCK-associated protein 1-like | HGNC:4862 |
| NDRG1 | N-myc downstream regulated 1 | HGNC:7679 |
| NDUFA4 | NADH dehydrogenase (ubiquinone) 1 alpha subcomplex, 4, | HGNC:7687 |
| | 9 kDa | |
| NDUFA4L2 | NADH dehydrogenase (ubiquinone) 1 alpha subcomplex, 4- | HGNC:29836 |
| | like 2 | |
| NDUFC2 | NADH dehydrogenase (ubiquinone) 1, subcomplex unknown, | HGNC:7706 |
| | 2, 14.5 kDa | |
| NDUFS5 | NADH dehydrogenase (ubiquinone) Fe—S protein 5, 15 kDa | HGNC:7712 |
| | (NADH-coenzyme Q reductase) | |
| NEFH | neurofilament, heavy polypeptide | HGNC:7737 |
| NEFL | neurofilament, light polypeptide | HGNC:7739 |
| NEGR1 | neuronal growth regulator 1 | HGNC:17302 |
| NETO1 | neuropilin (NRP) and tolloid (TLL)-like 1 | HGNC:13823 |
| NETO2 | neuropilin (NRP) and tolloid (TLL)-like 2 | HGNC:14644 |
| NEXN | nexilin (F actin binding protein) | HGNC:29557 |
| NFKBIZ | nuclear factor of kappa light polypeptide gene enhancer in B- | HGNC:29805 |
| | cells inhibitor, zeta | |
| NID1 | nidogen 1 | HGNC:7821 |
| NIT2 | nitrilase family, member 2 | HGNC:29878 |
| NKG7 | natural killer cell group 7 sequence | HGNC:7830 |
| NLRP10 | NLR family, pyrin domain containing 10 | HGNC:21464 |
| NOP16 | NOP16 nucleolar protein homolog (yeast) | HGNC:26934 |
| NOTCH1 | notch 1 | HGNC:7881 |
| NOVA1 | neuro-oncological ventral antigen 1 | HGNC:7886 |
| NOX4 | NADPH oxidase 4 | HGNC:7891 |
| NPC1 | Niemann-Pick disease, type C1 | HGNC:7897 |
| NPL | N-acetylneuraminate pyruvate lyase (dihydrodipicolinate | HGNC:16781 |
| | synthase) | |
| NPR1 | natriuretic peptide receptor A/guanylate cyclase A | HGNC:7943 |
| | (atrionatriuretic peptide receptor A) | |
| NPR2 | natriuretic peptide receptor B/guanylate cyclase B | HGNC:7944 |
| | (atrionatriuretic peptide receptor B) | |
| NQO2 | NAD(P)H dehydrogenase, quinone 2 | HGNC:7856 |
| NR1H3 | nuclear receptor subfamily 1, group H, member 3 | HGNC:7966 |
| NRN1 | neuritin 1 | HGNC:17972 |
| NRP2 | neuropilin 2 | HGNC:8005 |
| NSDHL | NAD(P) dependent steroid dehydrogenase-like | HGNC:13398 |
| NTAN1 | N-terminal asparagine amidase | HGNC:29909 |
| NTM | neurotrimin | HGNC:17941 |
| NTN1 | netrin 1 | HGNC:8029 |
| NTRK2 | neurotrophic tyrosine kinase, receptor, type 2 | HGNC:8032 |
| NUDT4 | nudix (nucleoside diphosphate linked moiety X)-type motif 4 | HGNC:8051 |
| NUS1 | nuclear undecaprenyl pyrophosphate synthase 1 homolog (S. cerevisiae) | HGNC:21042 |
| NXF3 | nuclear RNA export factor 3 | HGNC:8073 |
| NXPH4 | neurexophilin 4 | HGNC:8078 |
| OAS1 | 2′-5′-oligoadenylate synthetase 1, 40/46 kDa | HGNC:8086 |
| OAS2 | 2′-5′-oligoadenylate synthetase 2, 69/71 kDa | HGNC:8087 |
| OASL | 2′-5′-oligoadenylate synthetase-like | HGNC:8090 |
| OC90 | otoconin 90 | HGNC:8100 |
| OCA2 | oculocutaneous albinism II | HGNC:8101 |
| ODZ3 | odz, odd Oz/ten-m homolog 3 (Drosophila) | HGNC:29944 |
| OLAH | oleoyl-ACP hydrolase | HGNC:25625 |
| OLFM4 | olfactomedin 4 | HGNC:17190 |
| OLFML3 | olfactomedin-like 3 | HGNC:24956 |
| OLIG3 | oligodendrocyte transcription factor 3 | HGNC:18003 |
| OR52E8 | olfactory receptor, family 52, subfamily E, member 8 | HGNC:15217 |
| OR7E24 | olfactory receptor, family 7, subfamily E, member 24 | HGNC:8396 |
| OSGIN2 | oxidative stress induced growth inhibitor family member 2 | HGNC:1355 |
| OTUB2 | OTU domain, ubiquitin aldehyde binding 2 | HGNC:20351 |
| OXCT2 | 3-oxoacid CoA transferase 2 | HGNC:18606 |
| P2RY12 | purinergic receptor P2Y, G-protein coupled, 12 | HGNC:18124 |
| P2RY4 | pyrimidinergic receptor P2Y, G-protein coupled, 4 | HGNC:8542 |
| PADI3 | peptidyl arginine deiminase, type III | HGNC:18337 |
| PADI4 | peptidyl arginine deiminase, type IV | HGNC:18368 |
| PAFAH2 | platelet-activating factor acetylhydrolase 2, 40 kDa | HGNC:8579 |
| PALM2- | PALM2-AKAP2 readthrough | HGNC:33529 |
| AKAP2 | | |
| PAM | peptidylglycine alpha-amidating monooxygenase | HGNC:8596 |
| PAMR1 | peptidase domain containing associated with muscle regeneration 1 | HGNC:24554 |
| PANK1 | pantothenate kinase 1 | HGNC:8598 |
| PAPLN | papilin, proteoglycan-like sulfated glycoprotein | HGNC:19262 |
| PAQR3 | progestin and adipoQ receptor family member III | HGNC:30130 |
| PARP14 | poly (ADP-ribose) polymerase family, member 14 | HGNC:29232 |
| PARVB | parvin, beta | HGNC:14653 |
| PC | pyruvate carboxylase | HGNC:8636 |
| PCCB | propionyl CoA carboxylase, beta polypeptide | HGNC:8654 |
| PCDH12 | protocadherin 12 | HGNC:8657 |
| PCDH18 | protocadherin 18 | HGNC:14268 |
| PCDH9 | protocadherin 9 | HGNC:8661 |
| PCK1 | phosphoenolpyruvate carboxykinase 1 (soluble) | HGNC:8724 |
| PCK2 | phosphoenolpyruvate carboxykinase 2 (mitochondrial) | HGNC:8725 |
| PCOLCE2 | procollagen C-endopeptidase enhancer 2 | HGNC:8739 |
| PCP4 | Purkinje cell protein 4 | HGNC:8742 |
| PCP4L1 | Purkinje cell protein 4 like 1 | HGNC:20448 |
| PCSK2 | proprotein convertase subtilisin/kexin type 2 | HGNC:8744 |
| PCSK6 | proprotein convertase subtilisin/kexin type 6 | HGNC:8569 |
| PCTP | phosphatidylcholine transfer protein | HGNC:8752 |
| PCYT2 | phosphate cytidylyltransferase 2, ethanolamine | HGNC:8756 |
| PDCD1 | programmed cell death 1 | HGNC:8760 |
| PDCD1LG2 | programmed cell death 1 ligand 2 | HGNC:18731 |
| PDE2A | phosphodiesterase 2A, cGMP-stimulated | HGNC:8777 |
| PDE3B | phosphodiesterase 3B, cGMP-inhibited | HGNC:8779 |
| PDE4DIP | phosphodiesterase 4D interacting protein | HGNC:15580 |
| PDE6A | phosphodiesterase 6A, cGMP-specific, rod, alpha | HGNC:8785 |
| PDGFC | platelet derived growth factor C | HGNC:8801 |
| PDGFD | platelet derived growth factor D | HGNC:30620 |
| PDGFRA | platelet-derived growth factor receptor, alpha polypeptide | HGNC:8803 |
| PDGFRB | platelet-derived growth factor receptor, beta polypeptide | HGNC:8804 |
| PDHA1 | pyruvate dehydrogenase (lipoamide) alpha 1 | HGNC:8806 |
| PDHX | pyruvate dehydrogenase complex, component X | HGNC:21350 |
| PDLIM3 | PDZ and LIM domain 3 | HGNC:20767 |
| PDZD4 | PDZ domain containing 4 | HGNC:21167 |
| PDZK1 | PDZ domain containing 1 | HGNC:8821 |
| PDZRN4 | PDZ domain containing ring finger 4 | HGNC:30552 |
| PEBP4 | phosphatidylethanolamine-binding protein 4 | HGNC:28319 |
| PECR | peroxisomal trans-2-enoyl-CoA reductase | HGNC:18281 |
| PENK | proenkephalin | HGNC:8831 |
| PEX11A | peroxisomal biogenesis factor 11 alpha | HGNC:8852 |
| PEX16 | peroxisomal biogenesis factor 16 | HGNC:8857 |
| PEX3 | peroxisomal biogenesis factor 3 | HGNC:8858 |
| PFKFB1 | 6-phosphofructo-2-kinase/fructose-2,6-biphosphatase 1 | HGNC:8872 |
| PFKFB4 | 6-phosphofructo-2-kinase/fructose-2,6-biphosphatase 4 | HGNC:8875 |
| PFN2 | profilin 2 | HGNC:8882 |
| PGBD5 | piggyBac transposable element derived 5 | HGNC:19405 |
| PGD | phosphogluconate dehydrogenase | HGNC:8891 |
| PGM1 | phosphoglucomutase 1 | HGNC:8905 |
| PGM5 | phosphoglucomutase 5 | HGNC:8908 |
| PGRMC1 | progesterone receptor membrane component 1 | HGNC:16090 |
| PHLDA2 | pleckstrin homology-like domain, family A, member 2 | HGNC:12385 |
| PHLDB1 | pleckstrin homology-like domain, family B, member 1 | HGNC:23697 |
| PHLDB2 | pleckstrin homology-like domain, family B, member 2 | HGNC:29573 |
| PHYHIP | phytanoyl-CoA 2-hydroxylase interacting protein | HGNC:16865 |
| PI15 | peptidase inhibitor 15 | HGNC:8946 |
| PI16 | peptidase inhibitor 16 | HGNC:21245 |
| PID1 | phosphotyrosine interaction domain containing 1 | HGNC:26084 |
| PIP | prolactin-induced protein | HGNC:8993 |
| PIRT | phosphoinositide-interacting regulator of transient receptor potential channels | HGNC:37239 |
| PKDCC | protein kinase domain containing, cytoplasmic homolog (mouse) | HGNC:25123 |
| PKIB | protein kinase (cAMP-dependent, catalytic) inhibitor beta | HGNC:9018 |
| PKLR | pyruvate kinase, liver and RBC | HGNC:9020 |
| PLA2G2A | phospholipase A2, group IIA (platelets, synovial fluid) | HGNC:9031 |
| PLA2G2F | phospholipase A2, group IIF | HGNC:30040 |
| PLA2G7 | phospholipase A2, group VII (platelet-activating factor acetylhydrolase, | HGNC:9040 |
| | plasma) | |
| PLA2R1 | phospholipase A2 receptor 1, 180 kDa | HGNC:9042 |
| PLAU | plasminogen activator, urokinase | HGNC:9052 |
| PLCL2 | phospholipase C-like 2 | HGNC:9064 |
| PLD5 | phospholipase D family, member 5 | HGNC:26879 |
| PLEK | pleckstrin | HGNC:9070 |
| PLEKHH1 | pleckstrin homology domain containing, family H (with MyTH4 domain) | HGNC:17733 |
| | member 1 | |
| PLEKHO2 | pleckstrin homology domain containing, family O member 2 | HGNC:30026 |
| PLIN1 | perilipin 1 | HGNC:9076 |
| PLIN2 | perilipin 2 | HGNC:248 |
| PLIN4 | perilipin 4 | HGNC:29393 |
| PLIN5 | perilipin 5 | HGNC:33196 |
| PLN | phospholamban | HGNC:9080 |
| PLP1 | proteolipid protein 1 | HGNC:9086 |
| PLXDC1 | plexin domain containing 1 | HGNC:20945 |
| PLXNA4 | plexin A4 | HGNC:9102 |
| PLXND1 | plexin D1 | HGNC:9107 |
| PM20D1 | peptidase M20 domain containing 1 | HGNC:26518 |
| PMEPA1 | prostate transmembrane protein, androgen induced 1 | HGNC:14107 |
| PMFBP1 | polyamine modulated factor 1 binding protein 1 | HGNC:17728 |
| PMM1 | phosphomannomutase 1 | HGNC:9114 |
| PMVK | phosphomevalonate kinase | HGNC:9141 |
| PNLDC1 | poly(A)-specific ribonuclease (PARN)-like domain containing 1 | HGNC:21185 |
| PNLIPRP2 | pancreatic lipase-related protein 2 | HGNC:9157 |
| PNLIPRP3 | pancreatic lipase-related protein 3 | HGNC:23492 |
| PNMAL1 | paraneoplastic Ma antigen family-like 1 | HGNC:25578 |
| PNPLA3 | patatin-like phospholipase domain containing 3 | HGNC:18590 |
| PNPLA5 | patatin-like phospholipase domain containing 5 | HGNC:24888 |
| PODN | podocan | HGNC:23174 |
| PODXL2 | podocalyxin-like 2 | HGNC:17936 |
| PON3 | paraoxonase 3 | HGNC:9206 |
| POPDC2 | popeye domain containing 2 | HGNC:17648 |
| POR | P450 (cytochrome) oxidoreductase | HGNC:9208 |
| POSTN | periostin, osteoblast specific factor | HGNC:16953 |
| POU3F1 | POU class 3 homeobox 1 | HGNC:9214 |
| PPAP2B | phosphatidic acid phosphatase type 2B | HGNC:9229 |
| PPARG | peroxisome proliferator-activated receptor gamma | HGNC:9236 |
| PPARGC1A | peroxisome proliferator-activated receptor gamma, coactivator 1 alpha | HGNC:9237 |
| PPBP | pro-platelet basic protein (chemokine (C—X—C motif) ligand 7) | HGNC:9240 |
| PPIF | peptidylprolyl isomerase F | HGNC:9259 |
| PPM1K | protein phosphatase, Mg2+/Mn2+ dependent, 1K | HGNC:25415 |
| PPP1R12B | protein phosphatase 1, regulatory subunit 12B | HGNC:7619 |
| PPP1R1A | protein phosphatase 1, regulatory (inhibitor) subunit 1A | HGNC:9286 |
| PPP1R3G | protein phosphatase 1, regulatory subunit 3G | HGNC:14945 |
| PPP2R2B | protein phosphatase 2, regulatory subunit B, beta | HGNC:9305 |
| PPP2R2C | protein phosphatase 2, regulatory subunit B, gamma | HGNC:9306 |
| PPP2R4 | protein phosphatase 2A activator, regulatory subunit 4 | HGNC:9308 |
| PPP4R4 | protein phosphatase 4, regulatory subunit 4 | HGNC:23788 |
| PPT2 | palmitoyl-protein thioesterase 2 | HGNC:9326 |
| PRAME | preferentially expressed antigen in melanoma | HGNC:9336 |
| PRB1 | proline-rich protein BstNI subfamily 1 | HGNC:9337 |
| PRB2 | proline-rich protein BstNI subfamily 2 | HGNC:9338 |
| PRB3 | proline-rich protein BstNI subfamily 3 | HGNC:9339 |
| PRDX2 | peroxiredoxin 2 | HGNC:9353 |
| PRELP | proline/arginine-rich end leucine-rich repeat protein | HGNC:9357 |
| PRF1 | perforin 1 (pore forming protein) | HGNC:9360 |
| PRG4 | proteoglycan 4 | HGNC:9364 |
| PRKAR2B | protein kinase, cAMP-dependent, regulatory, type II, beta | HGNC:9392 |
| PRKCB | protein kinase C, beta | HGNC:9395 |
| PRKCQ | protein kinase C, theta | HGNC:9410 |
| PRR4 | proline rich 4 (lacrimal) | HGNC:18020 |
| PRR5L | proline rich 5 like | HGNC:25878 |
| PRRT4 | proline-rich transmembrane protein 4 | HGNC:37280 |
| PRSS22 | protease, serine, 22 | HGNC:14368 |
| PRSS27 | protease, serine 27 | HGNC:15475 |
| PRSS8 | protease, serine, 8 | HGNC:9491 |
| PRUNE2 | prune homolog 2 (Drosophila) | HGNC:25209 |
| PSAPL1 | prosaposin-like 1 (gene/pseudogene) | HGNC:33131 |
| PSG4 | pregnancy specific beta-1-glycoprotein 4 | HGNC:9521 |
| PSMA4 | proteasome (prosome, macropain) subunit, alpha type, 4 | HGNC:9533 |
| PSMB9 | proteasome (prosome, macropain) subunit, beta type, 9 (large multifunctional | HGNC:9546 |
| | peptidase 2) | |
| PSME4 | proteasome (prosome, macropain) activator subunit 4 | HGNC:20635 |
| PSPH | phosphoserine phosphatase | HGNC:9577 |
| PTGDS | prostaglandin D2 synthase 21 kDa (brain) | HGNC:9592 |
| PTGER3 | prostaglandin E receptor 3 (subtype EP3) | HGNC:9595 |
| PTGFR | prostaglandin F receptor (FP) | HGNC:9600 |
| PTGIS | prostaglandin I2 (prostacyclin) synthase | HGNC:9603 |
| PTHLH | parathyroid hormone-like hormone | HGNC:9607 |
| PTN | pleiotrophin | HGNC:9630 |
| PTPN7 | protein tyrosine phosphatase, non-receptor type 7 | HGNC:9659 |
| PTPRC | protein tyrosine phosphatase, receptor type, C | HGNC:9666 |
| PTPRCAP | protein tyrosine phosphatase, receptor type, C-associated protein | HGNC:9667 |
| PTPRG | protein tyrosine phosphatase, receptor type, G | HGNC:9671 |
| PTX3 | pentraxin 3, long | HGNC:9692 |
| PXDN | peroxidasin homolog (Drosophila) | HGNC:14966 |
| PXMP2 | peroxisomal membrane protein 2, 22 kDa | HGNC:9716 |
| PXMP4 | peroxisomal membrane protein 4, 24 kDa | HGNC:15920 |
| PXN | paxillin | HGNC:9718 |
| PYHIN1 | pyrin and HIN domain family, member 1 | HGNC:28894 |
| RAB15 | RAB15, member RAS oncogene family | HGNC:20150 |
| RAC2 | ras-related C3 botulinum toxin substrate 2 (rho family, small GTP binding | HGNC:9802 |
| | protein Rac2) | |
| RAC3 | ras-related C3 botulinum toxin substrate 3 (rho family, small GTP binding | HGNC:9803 |
| | protein Rac3) | |
| RAMP2 | receptor (G protein-coupled) activity modifying protein 2 | HGNC:9844 |
| RARRES1 | retinoic acid receptor responder (tazarotene induced) 1 | HGNC:9867 |
| RARRES2 | retinoic acid receptor responder (tazarotene induced) 2 | HGNC:9868 |
| RASA3 | RAS p21 protein activator 3 | HGNC:20331 |
| RASAL1 | RAS protein activator like 1 (GAP1 like) | HGNC:9873 |
| RASEF | RAS and EF-hand domain containing | HGNC:26464 |
| RASSF3 | Ras association (RalGDS/AF-6) domain family member 3 | HGNC:14271 |
| RBM47 | RNA binding motif protein 47 | HGNC:30358 |
| RBMS3 | RNA binding motif, single stranded interacting protein 3 | HGNC:13427 |
| RBP1 | retinol binding protein 1, cellular | HGNC:9919 |
| RBP4 | retinol binding protein 4, plasma | HGNC:9922 |
| RBP5 | retinol binding protein 5, cellular | HGNC:15847 |
| RCL1 | RNA terminal phosphate cyclase-like 1 | HGNC:17687 |
| RDH11 | retinol dehydrogenase 11 (all-trans/9-cis/11-cis) | HGNC:17964 |
| RDH5 | retinol dehydrogenase 5 (11-cis/9-cis) | HGNC:9940 |
| RECK | reversion-inducing-cysteine-rich protein with kazal motifs | HGNC:11345 |
| REEP6 | receptor accessory protein 6 | HGNC:30078 |
| REG3G | regenerating islet-derived 3 gamma | HGNC:29595 |
| RETSAT | retinol saturase (all-trans-retinol 13,14-reductase) | HGNC:25991 |
| RFTN1 | raftlin, lipid raft linker 1 | HGNC:30278 |
| RGL1 | ral guanine nucleotide dissociation stimulator-like 1 | HGNC:30281 |
| RGS9BP | regulator of G protein signaling 9 binding protein | HGNC:30304 |
| RHCG | Rh family, C glycoprotein | HGNC:18140 |
| RHOXF2 | Rhox homeobox family, member 2 | HGNC:30011 |
| RIMBP2 | RIMS binding protein 2 | HGNC:30339 |
| RIMS4 | regulating synaptic membrane exocytosis 4 | HGNC:16183 |
| RNASE1 | ribonuclease, RNase A family, 1 (pancreatic) | HGNC:10044 |
| RND3 | Rho family GTPase 3 | HGNC:671 |
| RNF133 | ring finger protein 133 | HGNC:21154 |
| RNF157 | ring finger protein 157 | HGNC:29402 |
| RNF17 | ring finger protein 17 | HGNC:10060 |
| RNFT1 | ring finger protein, transmembrane 1 | HGNC:30206 |
| ROBO3 | roundabout, axon guidance receptor, homolog 3 (Drosophila) | HGNC:13433 |
| ROBO4 | roundabout, axon guidance receptor, homolog 4 (Drosophila) | HGNC:17985 |
| ROS1 | c-ros oncogene 1, receptor tyrosine kinase | HGNC:10261 |
| RRAS | related RAS viral (r-ras) oncogene homolog | HGNC:10447 |
| RSAD2 | radical S-adenosyl methionine domain containing 2 | HGNC:30908 |
| RSPO3 | R-spondin 3 | HGNC:20866 |
| RTN1 | reticulon 1 | HGNC:10467 |
| S100A2 | S100 calcium binding protein A2 | HGNC:10492 |
| S100A3 | S100 calcium binding protein A3 | HGNC:10493 |
| S100A7 | S100 calcium binding protein A7 | HGNC:10497 |
| S100A7A | S100 calcium binding protein A7A | HGNC:21657 |
| S100A8 | S100 calcium binding protein A8 | HGNC:10498 |
| S100A9 | S100 calcium binding protein A9 | HGNC:10499 |
| S100P | S100 calcium binding protein P | HGNC:10504 |
| S1PR4 | sphingosine-1-phosphate receptor 4 | HGNC:3170 |
| SAA1 | serum amyloid A1 | HGNC:10513 |
| SAMD3 | sterile alpha motif domain containing 3 | HGNC:21574 |
| SAMD5 | sterile alpha motif domain containing 5 | HGNC:21180 |
| SAMD9L | sterile alpha motif domain containing 9-like | HGNC:1349 |
| SAMHD1 | SAM domain and HD domain 1 | HGNC:15925 |
| SAMM50 | sorting and assembly machinery component 50 homolog (S. cerevisiae) | HGNC:24276 |
| SAMSN1 | SAM domain, SH3 domain and nuclear localization signals 1 | HGNC:10528 |
| SASH3 | SAM and SH3 domain containing 3 | HGNC:15975 |
| SATB2 | SATB homeobox 2 | HGNC:21637 |
| SCSDL | sterol-C5-desaturase (ERG3 delta-5-desaturase homolog, S. cerevisiae)-like | HGNC:10547 |
| SCAP | SREBF chaperone | HGNC:30634 |
| SCARA5 | scavenger receptor class A, member 5 (putative) | HGNC:28701 |
| SCGB1D2 | secretoglobin, family 1D, member 2 | HGNC:18396 |
| SCGB2A1 | secretoglobin, family 2A, member 1 | HGNC:7051 |
| SCGB2A2 | secretoglobin, family 2A, member 2 | HGNC:7050 |
| SCGB3A1 | secretoglobin, family 3A, member 1 | HGNC:18384 |
| SCN2A | sodium channel, voltage-gated, type II, alpha subunit | HGNC:10588 |
| SCN7A | sodium channel, voltage-gated, type VII, alpha subunit | HGNC:10594 |
| SCUBE2 | signal peptide, CUB domain, EGF-like 2 | HGNC:30425 |
| SDPR | serum deprivation response | HGNC:10690 |
| SEC11C | SEC11 homolog C (S. cerevisiae) | HGNC:23400 |
| SEC14L2 | SEC14-like 2 (S. cerevisiae) | HGNC:10699 |
| SEC14L4 | SEC14-like 4 (S. cerevisiae) | HGNC:20627 |
| SELPLG | selectin P ligand | HGNC:10722 |
| SEMA3A | sema domain, immunoglobulin domain (Ig), short basic domain, secreted, | HGNC:10723 |
| | (semaphorin) 3A | |
| SEMA5B | sema domain, seven thrombospondin repeats (type 1 and type 1-like), | HGNC:10737 |
| | transmembrane domain (TM) and short cytoplasmic domain, (semaphorin) 5B | |
| SEMA6B | sema domain, transmembrane domain (TM), and cytoplasmic domain, | HGNC:10739 |
| | (semaphorin) 6B | |
| SEMA7A | semaphorin 7A, GPI membrane anchor (John Milton Hagen blood group) | HGNC:10741 |
| SERINC2 | serine incorporator 2 | HGNC:23231 |
| SERPINA12 | serpin peptidase inhibitor, clade A (alpha-1 antiproteinase, antitrypsin), member | HGNC:18359 |
| | 12 | |
| SERPINA3 | serpin peptidase inhibitor, clade A (alpha-1 antiproteinase, antitrypsin), member 3 | HGNC:16 |
| SERPINA4 | serpin peptidase inhibitor, clade A (alpha-1 antiproteinase, antitrypsin), member 4 | HGNC:8948 |
| SERPINA9 | serpin peptidase inhibitor, clade A (alpha-1 antiproteinase, antitrypsin), member 9 | HGNC:15995 |
| SERPINB13 | serpin peptidase inhibitor, clade B (ovalbumin), member 13 | HGNC:8944 |
| SERPINB3 | serpin peptidase inhibitor, clade B (ovalbumin), member 3 | HGNC:10569 |
| SERPINB7 | serpin peptidase inhibitor, clade B (ovalbumin), member 7 | HGNC:13902 |
| SERPINE2 | serpin peptidase inhibitor, clade E (nexin, plasminogen activator inhibitor type | HGNC:8951 |
| | 1), member 2 | |
| SERPINF1 | serpin peptidase inhibitor, clade F (alpha-2 antiplasmin, pigment epithelium | HGNC:8824 |
| | derived factor), member 1 | |
| SERPING1 | serpin peptidase inhibitor, clade G (C1 inhibitor), member 1 | HGNC:1228 |
| SETBP1 | SET binding protein 1 | HGNC:15573 |
| SFRP5 | secreted frizzled-related protein 5 | HGNC:10779 |
| SGCD | sarcoglycan, delta (35 kDa dystrophin-associated glycoprotein) | HGNC:10807 |
| SGCG | sarcoglycan, gamma (35 kDa dystrophin-associated glycoprotein) | HGNC:10809 |
| SGK2 | serum/glucocorticoid regulated kinase 2 | HGNC:13900 |
| SGPP2 | sphingosine-1-phosphate phosphatase 2 | HGNC:19953 |
| SH2B3 | SH2B adaptor protein 3 | HGNC:29605 |
| SH2D1A | SH2 domain containing 1A | HGNC:10820 |
| SH3KBP1 | SH3-domain kinase binding protein 1 | HGNC:13867 |
| SHF | Src homology 2 domain containing F | HGNC:25116 |
| SHISA2 | shisa homolog 2 (Xenopus laevis) | HGNC:20366 |
| SHISA6 | shisa homolog 6 (Xenopus laevis) | HGNC:34491 |
| SIGLEC1 | sialic acid binding Ig-like lectin 1, sialoadhesin | HGNC:11127 |
| SIPA1L2 | signal-induced proliferation-associated 1 like 2 | HGNC:23800 |
| SIT1 | signaling threshold regulating transmembrane adaptor 1 | HGNC:17710 |
| SLA | Src-like-adaptor | HGNC:10902 |
| SLAMF1 | signaling lymphocytic activation molecule family member 1 | HGNC:10903 |
| SLAMF6 | SLAM family member 6 | HGNC:21392 |
| SLAMF8 | SLAM family member 8 | HGNC:21391 |
| SLC11A2 | solute carrier family 11 (proton-coupled divalent metal ion transporters), | HGNC:10908 |
| | member 2 | |
| SLC12A8 | solute carrier family 12 (potassium/chloride transporters), member 8 | HGNC:15595 |
| SLC13A4 | solute carrier family 13 (sodium/sulfate symporters), member 4 | HGNC:15827 |
| SLC15A1 | solute carrier family 15 (oligopeptide transporter), member 1 | HGNC:10920 |
| SLC15A2 | solute carrier family 15 (H+/peptide transporter), member 2 | HGNC:10921 |
| SLC16A11 | solute carrier family 16, member 11 (monocarboxylic acid transporter 11) | HGNC:23093 |
| SLC16A6 | solute carrier family 16, member 6 (monocarboxylic acid transporter 7) | HGNC:10927 |
| SLC16A9 | solute carrier family 16, member 9 (monocarboxylic acid transporter 9) | HGNC:23520 |
| SLC24A5 | solute carrier family 24, member 5 | HGNC:20611 |
| SLC25 A1 | solute carrier family 25 (mitochondrial carrier; citrate transporter), member 1 | HGNC:10979 |
| SLC25 A11 | solute carrier family 25 (mitochondrial carrier; oxoglutarate carrier), member 11 | HGNC:10981 |
| SLC25A17 | solute carrier family 25 (mitochondrial carrier; peroxisomal membrane protein, | HGNC:10987 |
| | 34 kDa), member 17 | |
| SLC25A18 | solute carrier family 25 (glutamate carrier), member 18 | HGNC:10988 |
| SLC25A33 | solute carrier family 25 (pyrimidine nucleotide carrier), member 33 | HGNC:29681 |
| SLC25A34 | solute carrier family 25, member 34 | HGNC:27653 |
| SLC25A35 | solute carrier family 25, member 35 | HGNC:31921 |
| SLC25A41 | solute carrier family 25, member 41 | HGNC:28533 |
| SLC25A5 | solute carrier family 25 (mitochondrial carrier; adenine nucleotide translocator), | HGNC:10991 |
| | member 5 | |
| SLC26A2 | solute carrier family 26 (sulfate transporter), member 2 | HGNC:10994 |
| SLC26A3 | solute carrier family 26, member 3 | HGNC:3018 |
| SLC26A6 | solute carrier family 26, member 6 | HGNC:14472 |
| SLC26A9 | solute carrier family 26, member 9 | HGNC:14469 |
| SLC27A2 | solute carrier family 27 (fatty acid transporter), member 2 | HGNC:10996 |
| SLC27A4 | solute carrier family 27 (fatty acid transporter), member 4 | HGNC:10998 |
| SLC27A6 | solute carrier family 27 (fatty acid transporter), member 6 | HGNC:11000 |
| SLC2A1 | solute carrier family 2 (facilitated glucose transporter), member 1 | HGNC:11005 |
| SLC2A14 | solute carrier family 2 (facilitated glucose transporter), member 14 | HGNC:18301 |
| SLC2A9 | solute carrier family 2 (facilitated glucose transporter), member 9 | HGNC:13446 |
| SLC30A1 | solute carrier family 30 (zinc transporter), member 1 | HGNC:11012 |
| SLC30A10 | solute carrier family 30, member 10 | HGNC:25355 |
| SLC31A1 | solute carrier family 31 (copper transporters), member 1 | HGNC:11016 |
| SLC31A2 | solute carrier family 31 (copper transporters), member 2 | HGNC:11017 |
| SLC35A2 | solute carrier family 35 (UDP-galactose transporter), member A2 | HGNC:11022 |
| SLC35B1 | solute carrier family 35, member B1 | HGNC:20798 |
| SLC39A8 | solute carrier family 39 (zinc transporter), member 8 | HGNC:20862 |
| SLC3A1 | solute carrier family 3 (cystine, dibasic and neutral amino acid transporters, | HGNC:11025 |
| | activator of cystine, dibasic and neutral amino acid transport), member 1 | |
| SLC40A1 | solute carrier family 40 (iron-regulated transporter), member 1 | HGNC:10909 |
| SLC41A1 | solute carrier family 41, member 1 | HGNC:19429 |
| SLC43A2 | solute carrier family 43, member 2 | HGNC:23087 |
| SLC43A3 | solute carrier family 43, member 3 | HGNC:17466 |
| SLC44A3 | solute carrier family 44, member 3 | HGNC:28689 |
| SLC45A4 | solute carrier family 45, member 4 | HGNC:29196 |
| SLC46A3 | solute carrier family 46, member 3 | HGNC:27501 |
| SLC5A6 | solute carrier family 5 (sodium-dependent vitamin transporter), member 6 | HGNC:11041 |
| SLC6A14 | solute carrier family 6 (amino acid transporter), member 14 | HGNC:11047 |
| SLC6A17 | solute carrier family 6, member 17 | HGNC:31399 |
| SLC7A4 | solute carrier family 7 (orphan transporter), member 4 | HGNC:11062 |
| SLC7A5 | solute carrier family 7 (amino acid transporter light chain, L system), member 5 | HGNC:11063 |
| SLC9A2 | solute carrier family 9, subfamily A (NHE2, cation proton antiporter 2), | HGNC:11072 |
| | member 2 | |
| SLCO4C1 | solute carrier organic anion transporter family, member 4C1 | HGNC:23612 |
| SLFN5 | schlafen family member 5 | HGNC:28286 |
| SLIT2 | slit homolog 2 (Drosophila) | HGNC:11086 |
| SLIT3 | slit homolog 3 (Drosophila) | HGNC:11087 |
| SMAD3 | SMAD family member 3 | HGNC:6769 |
| SMAD6 | SMAD family member 6 | HGNC:6772 |
| SMOC1 | SPARC related modular calcium binding 1 | HGNC:20318 |
| SMOC2 | SPARC related modular calcium binding 2 | HGNC:20323 |
| SMOX | spermine oxidase | HGNC:15862 |
| SMTN | smoothelin | HGNC:11126 |
| SNCG | synuclein, gamma (breast cancer-specific protein 1) | HGNC:11141 |
| SNED1 | sushi, nidogen and EGF-like domains 1 | HGNC:24696 |
| SNX10 | sorting nexin 10 | HGNC:14974 |
| SNX4 | sorting nexin 4 | HGNC:11175 |
| SOAT1 | sterol O-acyltransferase 1 | HGNC:11177 |
| SOD3 | superoxide dismutase 3, extracellular | HGNC:11181 |
| SORBS1 | sorbin and SH3 domain containing 1 | HGNC:14565 |
| SORD | sorbitol dehydrogenase | HGNC:11184 |
| SOST | sclerostin | HGNC:13771 |
| SOSTDC1 | sclerostin domain containing 1 | HGNC:21748 |
| SOX7 | SRY (sex determining region Y)-box 7 | HGNC:18196 |
| SOX9 | SRY (sex determining region Y)-box 9 | HGNC:11204 |
| SP5 | Sp5 transcription factor | HGNC:14529 |
| SP8 | Sp8 transcription factor | HGNC:19196 |
| SPARC | secreted protein, acidic, cysteine-rich (osteonectin) | HGNC:11219 |
| SPEG | SPEG complex locus | HGNC:16901 |
| SPI1 | spleen focus forming virus (SFFV) proviral integration oncogene spi1 | HGNC:11241 |
| SPIRE2 | spire homolog 2 (Drosophila) | HGNC:30623 |
| SPNS2 | spinster homolog 2 (Drosophila) | HGNC:26992 |
| SPOCK1 | sparc/osteonectin, cwcv and kazal-like domains proteoglycan (testican) 1 | HGNC:11251 |
| SPOCK2 | sparc/osteonectin, cwcv and kazal-like domains proteoglycan (testican) 2 | HGNC:13564 |
| SPP1 | secreted phosphoprotein 1 | HGNC:11255 |
| SPRR1A | small proline-rich protein 1A | HGNC:11259 |
| SPRR1B | small proline-rich protein 1B | HGNC:11260 |
| SPRR2A | small proline-rich protein 2A | HGNC:11261 |
| SPRR2B | small proline-rich protein 2B | HGNC:11262 |
| SPRR2D | small proline-rich protein 2D | HGNC:11264 |
| SPRR4 | small proline-rich protein 4 | HGNC:23173 |
| SPRY1 | sprouty homolog 1, antagonist of FGF signaling (Drosophila) | HGNC:11269 |
| SPTBN5 | spectrin, beta, non-erythrocytic 5 | HGNC:15680 |
| SRD5A1 | steroid-5-alpha-reductase, alpha polypeptide 1 (3-oxo-5 alpha-steroid delta 4- | HGNC:11284 |
| | dehydrogenase alpha 1) | |
| SRD5A3 | steroid 5 alpha-reductase 3 | HGNC:25812 |
| SREBF1 | sterol regulatory element binding transcription factor 1 | HGNC:11289 |
| SRGN | serglycin | HGNC:9361 |
| SRMS | src-related kinase lacking C-terminal regulatory tyrosine and N-terminal | HGNC:11298 |
| | myristylation sites | |
| SRPRB | signal recognition particle receptor, B subunit | HGNC:24085 |
| SRPX | sushi-repeat containing protein, X-linked | HGNC:11309 |
| SRXN1 | sulfiredoxin 1 | HGNC:16132 |
| SSPN | sarcospan | HGNC:11322 |
| ST6GALNAC4 | ST6 (alpha-N-acetyl-neuraminyl-2,3-beta-galactosyl-1,3)-N- | HGNC:17846 |
| | acetylgalactosaminide alpha-2,6-sialyltransferase 4 | |
| STAB2 | stabilin 2 | HGNC:18629 |
| STAP2 | signal transducing adaptor family member 2 | HGNC:30430 |
| STARD4 | StAR-related lipid transfer (START) domain containing 4 | HGNC:18058 |
| STAT1 | signal transducer and activator of transcription 1, 91 kDa | HGNC:11362 |
| STAT5A | signal transducer and activator of transcription 5A | HGNC:11366 |
| STEAP4 | STEAP family member 4 | HGNC:21923 |
| STK17B | serine/threonine kinase 17b | HGNC:11396 |
| STRBP | spermatid perinuclear RNA binding protein | HGNC:16462 |
| STS | steroid sulfatase (microsomal), isozyme S | HGNC:11425 |
| STXBP5L | syntaxin binding protein 5-like | HGNC:30757 |
| SULT1C3 | sulfotransferase family, cytosolic, 1C, member 3 | HGNC:33543 |
| SULT4A1 | sulfotransferase family 4A, member 1 | HGNC:14903 |
| SUOX | sulfite oxidase | HGNC:11460 |
| SUSD4 | sushi domain containing 4 | HGNC:25470 |
| SVEP1 | sushi, von Willebrand factor type A, EGF and pentraxin domain | HGNC:15985 |
| | containing 1 | |
| SYN2 | synapsin II | HGNC:11495 |
| SYNE1 | spectrin repeat containing, nuclear envelope 1 | HGNC:17089 |
| SYNM | synemin, intermediate filament protein | HGNC:24466 |
| SYNPO | synaptopodin | HGNC:30672 |
| SYNPO2 | synaptopodin 2 | HGNC:17732 |
| SYPL2 | synaptophysin-like 2 | HGNC:27638 |
| SYT12 | synaptotagmin XII | HGNC:18381 |
| SYT4 | synaptotagmin IV | HGNC:11512 |
| SYT7 | synaptotagmin VII | HGNC:11514 |
| SYTL5 | synaptotagmin-like 5 | HGNC:15589 |
| TAC1 | tachykinin, precursor 1 | HGNC:11517 |
| TACC1 | transforming, acidic coiled-coil containing protein 1 | HGNC:11522 |
| TACR2 | tachykinin receptor 2 | HGNC:11527 |
| TAGLN | transgelin | HGNC:11553 |
| TAGLN3 | transgelin 3 | HGNC:29868 |
| TAP1 | transporter 1, ATP-binding cassette, sub-family B (MDR/TAP) | HGNC:43 |
| TAS1R3 | taste receptor, type 1, member 3 | HGNC:15661 |
| TBX5 | T-box 5 | HGNC:11604 |
| TCHH | trichohyalin | HGNC:11791 |
| TCHHL1 | trichohyalin-like 1 | HGNC:31796 |
| TDRD12 | tudor domain containing 12 | HGNC:25044 |
| TDRD6 | tudor domain containing 6 | HGNC:21339 |
| TECR | trans-2,3-enoyl-CoA reductase | HGNC:4551 |
| TECRL | trans-2,3-enoyl-CoA reductase-like | HGNC:27365 |
| TENC1 | tensin like C1 domain containing phosphatase (tensin 2) | HGNC:19737 |
| TEX264 | testis expressed 264 | HGNC:30247 |
| TF | transferrin | HGNC:11740 |
| TFF3 | trefoil factor 3 (intestinal) | HGNC:11757 |
| TFPI | tissue factor pathway inhibitor (lipoprotein-associated coagulation | HGNC:11760 |
| | inhibitor) | |
| TFRC | transferrin receptor (p90, CD71) | HGNC:11763 |
| TG | thyroglobulin | HGNC:11764 |
| TGFB1 | transforming growth factor, beta 1 | HGNC:11766 |
| TGFBR3 | transforming growth factor, beta receptor III | HGNC:11774 |
| TGM6 | transglutaminase 6 | HGNC:16255 |
| TGM7 | transglutaminase 7 | HGNC:30790 |
| THBS2 | thrombospondin 2 | HGNC:11786 |
| THEMIS | thymocyte selection associated | HGNC:21569 |
| THRSP | thyroid hormone responsive | HGNC:11800 |
| TIGIT | T cell immunoreceptor with Ig and ITIM domains | HGNC:26838 |
| TIMM8B | translocase of inner mitochondrial membrane 8 homolog B (yeast) | HGNC:11818 |
| TIMP3 | TIMP metallopeptidase inhibitor 3 | HGNC:11822 |
| TIMP4 | TIMP metallopeptidase inhibitor 4 | HGNC:11823 |
| TKT | transketolase | HGNC:11834 |
| TLCD1 | TLC domain containing 1 | HGNC:25177 |
| TLCD2 | TLC domain containing 2 | HGNC:33522 |
| TLE2 | transducin-like enhancer of split 2 (E(sp1) homolog, Drosophila) | HGNC:11838 |
| TLN1 | talin 1 | HGNC:11845 |
| TLN2 | talin 2 | HGNC:15447 |
| TM4SF1 | transmembrane 4 L six family member 1 | HGNC:11853 |
| TM7SF2 | transmembrane 7 superfamily member 2 | HGNC:11863 |
| TMC2 | transmembrane channel-like 2 | HGNC:16527 |
| TMC5 | transmembrane channel-like 5 | HGNC:22999 |
| TMC8 | transmembrane channel-like 8 | HGNC:20474 |
| TMCC3 | transmembrane and coiled-coil domain family 3 | HGNC:29199 |
| TMED3 | transmembrane emp24 protein transport domain containing 3 | HGNC:28889 |
| TMED5 | transmembrane emp24 protein transport domain containing 5 | HGNC:24251 |
| TMEM132C | transmembrane protein 132C | HGNC:25436 |
| TMEM135 | transmembrane protein 135 | HGNC:26167 |
| TMEM164 | transmembrane protein 164 | HGNC:26217 |
| TMEM171 | transmembrane protein 171 | HGNC:27031 |
| TMEM176A | transmembrane protein 176A | HGNC:24930 |
| TMEM176B | transmembrane protein 176B | HGNC:29596 |
| TMEM189 | transmembrane protein 189 | HGNC:16735 |
| TMEM33 | transmembrane protein 33 | HGNC:25541 |
| TMEM37 | transmembrane protein 37 | HGNC:18216 |
| TMEM41A | transmembrane protein 41A | HGNC:30544 |
| TMEM56 | transmembrane protein 56 | HGNC:26477 |
| TMEM63C | transmembrane protein 63C | HGNC:23787 |
| TMEM91 | transmembrane protein 91 | HGNC:32393 |
| TMEM97 | transmembrane protein 97 | HGNC:28106 |
| TMPRSS11E | transmembrane protease, serine 11E | HGNC:24465 |
| TNFAIP2 | tumor necrosis factor, alpha-induced protein 2 | HGNC:11895 |
| TNFRSF1B | tumor necrosis factor receptor superfamily, member 1B | HGNC:11917 |
| TNMD | tenomodulin | HGNC:17757 |
| TNNT1 | troponin T type 1 (skeletal, slow) | HGNC:11948 |
| TNNT2 | troponin T type 2 (cardiac) | HGNC:11949 |
| TNR | tenascin R (restrictin, janusin) | HGNC:11953 |
| TNS1 | tensin 1 | HGNC:11973 |
| TNS3 | tensin 3 | HGNC:21616 |
| TNXB | tenascin XB | HGNC:11976 |
| TP53INP2 | tumor protein p53 inducible nuclear protein 2 | HGNC:16104 |
| TPD52L1 | tumor protein D52-like 1 | HGNC:12006 |
| TPM2 | tropomyosin 2 (beta) | HGNC:12011 |
| TPSAB1 | tryptase alpha/beta 1 | HGNC:12019 |
| TPSB2 | tryptase beta 2 (gene/pseudogene) | HGNC:14120 |
| TRANK1 | tetratricopeptide repeat and ankyrin repeat containing 1 | HGNC:29011 |
| TRAT1 | T cell receptor associated transmembrane adaptor 1 | HGNC:30698 |
| TRIL | TLR4 interactor with leucine-rich repeats | HGNC:22200 |
| TRIM17 | tripartite motif containing 17 | HGNC:13430 |
| TRIM2 | tripartite motif containing 2 | HGNC:15974 |
| TRIM22 | tripartite motif containing 22 | HGNC:16379 |
| TRIM48 | tripartite motif containing 48 | HGNC:19021 |
| TRIM55 | tripartite motif containing 55 | HGNC:14215 |
| TRIM58 | tripartite motif containing 58 | HGNC:24150 |
| TRIM67 | tripartite motif containing 67 | HGNC:31859 |
| TRIML2 | tripartite motif family-like 2 | HGNC:26378 |
| TRIO | triple functional domain (PTPRF interacting) | HGNC:12303 |
| TRPS1 | trichorhinophalangeal syndrome I | HGNC:12340 |
| TSHZ2 | teashirt zinc finger homeobox 2 | HGNC:13010 |
| TSPAN2 | tetraspanin 2 | HGNC:20659 |
| TSPAN6 | tetraspanin 6 | HGNC:11858 |
| TST | thiosulfate sulfurtransferase (rhodanese) | HGNC:12388 |
| TTC38 | tetratricopeptide repeat domain 38 | HGNC:26082 |
| TTC39B | tetratricopeptide repeat domain 39B | HGNC:23704 |
| TTLL4 | tubulin tyrosine ligase-like family, member 4 | HGNC:28976 |
| TTYH3 | tweety homolog 3 (Drosophila) | HGNC:22222 |
| TUSC5 | tumor suppressor candidate 5 | HGNC:29592 |
| TXN | thioredoxin | HGNC:12435 |
| TXNDC9 | thioredoxin domain containing 9 | HGNC:24110 |
| TYR | tyrosinase (oculocutaneous albinism IA) | HGNC:12442 |
| TYRP1 | tyrosinase-related protein 1 | HGNC:12450 |
| UBA7 | ubiquitin-like modifier activating enzyme 7 | HGNC:12471 |
| UBASH3A | ubiquitin associated and SH3 domain containing A | HGNC:12462 |
| UBD | ubiquitin D | HGNC:18795 |
| UBE2L6 | ubiquitin-conjugating enzyme E2L 6 | HGNC:12490 |
| UBE2QL1 | ubiquitin-conjugating enzyme E2Q family-like 1 | HGNC:37269 |
| UBE2S | ubiquitin-conjugating enzyme E2S | HGNC:17895 |
| UBIAD1 | UbiA prenyltransferase domain containing 1 | HGNC:30791 |
| UBR4 | ubiquitin protein ligase E3 component n-recognin 4 | HGNC:30313 |
| UGT1A4 | UDP glucuronosyltransferase 1 family, polypeptide A4 | HGNC:12536 |
| UGT1A6 | UDP glucuronosyltransferase 1 family, polypeptide A6 | HGNC:12538 |
| UGT2A1 | UDP glucuronosyltransferase 2 family, polypeptide A1, complex | HGNC:12542 |
| | locus | |
| UGT2A2 | UDP glucuronosyltransferase 2 family, polypeptide A2 | HGNC:28183 |
| UGT3A2 | UDP glycosyltransferase 3 family, polypeptide A2 | HGNC:27266 |
| ULK4 | unc-51-like kinase 4 (C. elegans) | HGNC:15784 |
| UNC5B | unc-5 homolog B (C. elegans) | HGNC:12568 |
| UPB1 | ureidopropionase, beta | HGNC:16297 |
| UQCRC1 | ubiquinol-cytochrome c reductase core protein I | HGNC:12585 |
| UQCRH | ubiquinol-cytochrome c reductase hinge protein | HGNC:12590 |
| UQCRQ | ubiquinol-cytochrome c reductase, complex III subunit VII, 9.5 kDa | HGNC:29594 |
| USMG5 | up-regulated during skeletal muscle growth 5 homolog (mouse) | HGNC:30889 |
| VCAM1 | vascular cell adhesion molecule 1 | HGNC:12663 |
| VGLL3 | vestigial like 3 (Drosophila) | HGNC:24327 |
| VIP | vasoactive intestinal peptide | HGNC:12693 |
| VIT | vitrin | HGNC:12697 |
| VLDLR | very low density lipoprotein receptor | HGNC:12698 |
| VNN1 | vanin 1 | HGNC:12705 |
| VNN2 | vanin 2 | HGNC:12706 |
| VSIG4 | V-set and immunoglobulin domain containing 4 | HGNC:17032 |
| VSNL1 | visinin-like 1 | HGNC:12722 |
| VTCN1 | V-set domain containing T cell activation inhibitor 1 | HGNC:28873 |
| VWF | von Willebrand factor | HGNC:12726 |
| WASF3 | WAS protein family, member 3 | HGNC:12734 |
| WDFY4 | WDFY family member 4 | HGNC:29323 |
| WDR72 | WD repeat domain 72 | HGNC:26790 |
| WFDC5 | WAP four-disulfide core domain 5 | HGNC:20477 |
| WFDC6 | WAP four-disulfide core domain 6 | HGNC:16164 |
| WIPF1 | WAS/WASL interacting protein family, member 1 | HGNC:12736 |
| WNT3 | wingless-type MMTV integration site family, member 3 | HGNC:12782 |
| WNT4 | wingless-type MMTV integration site family, member 4 | HGNC:12783 |
| WNT7B | wingless-type MMTV integration site family, member 7B | HGNC:12787 |
| WNT9B | wingless-type MMTV integration site family, member 9B | HGNC:12779 |
| WSCD2 | WSC domain containing 2 | HGNC:29117 |
| XAGE5 | X antigen family, member 5 | HGNC:30930 |
| XBP1 | X-box binding protein 1 | HGNC:12801 |
| XPNPEP2 | X-prolyl aminopeptidase (aminopeptidase P) 2, membrane-bound | HGNC:12823 |
| XYLB | xylulokinase homolog (H. influenzae) | HGNC:12839 |
| ZBED2 | zinc finger, BED-type containing 2 | HGNC:20710 |
| ZC3H12A | zinc finger CCCH-type containing 12A | HGNC:26259 |
| ZCCHC24 | zinc finger, CCHC domain containing 24 | HGNC:26911 |
| ZEB1 | zinc finger E-box binding homeobox 1 | HGNC:11642 |
| ZFHX3 | zinc finger homeobox 3 | HGNC:777 |
| ZFHX4 | zinc finger homeobox 4 | HGNC:30939 |
| ZFPM2 | zinc finger protein, multitype 2 | HGNC:16700 |
| ZG16B | zymogen granule protein 16 homolog B (rat) | HGNC:30456 |
| ZIC1 | Zic family member 1 | HGNC:12872 |
| ZIC4 | Zic family member 4 | HGNC:20393 |
| ZNF117 | zinc finger protein 117 | HGNC:12897 |
| ZNF254 | zinc finger protein 254 | HGNC:13047 |
| ZNF385B | zinc finger protein 385B | HGNC:26332 |
| ZNF43 | zinc finger protein 43 | HGNC:13109 |
| ZNF493 | zinc finger protein 493 | HGNC:23708 |
| ZNF703 | zinc finger protein 703 | HGNC:25883 |
| ZNF80 | zinc finger protein 80 | HGNC:13155 |
| ZNF831 | zinc finger protein 831 | HGNC:16167 |
| ZP1 | zona pellucida glycoprotein 1 (sperm receptor) | HGNC:13187 |
| FAM213A | family with sequence similarity 213, member A | HGNC:28651 |
| OTOGL | otogelin-like | HGNC:26901 |
| SERTM1 | serine-rich and transmembrane domain containing 1 | HGNC:33792 |
| MFSD12 | major facilitator superfamily domain containing 12 | HGNC:28299 |
| RUSC1-AS1 | RUSC1 antisense RNA 1 (non-protein coding) | HGNC:26680 |
| SH3D21 | SH3 domain containing 21 | HGNC:26236 |
| AXDND1 | axonemal dynein light chain domain containing 1 | HGNC:26564 |
| LAMP5 | lysosomal-associated membrane protein family, member 5 | HGNC:16097 |
| METTL19 | methyltransferase like 19 | HGNC:26653 |
| NDNF | neuron-derived neurotrophic factor | HGNC:26256 |
| ADTRP | androgen-dependent TFPI-regulating protein | HGNC:21214 |
| CLPSL2 | colipase-like 2 | HGNC:21250 |
| SAPCD1 | suppressor APC domain containing 1 | HGNC:13938 |
| ZC2HC1B | zinc finger, C2HC-type containing 1B | HGNC:21174 |
| HILPDA | hypoxia inducible lipid droplet-associated | HGNC:28859 |
| SAPCD2 | suppressor APC domain containing 2 | HGNC:28055 |
| CACFD1 | calcium channel flower domain containing 1 | HGNC:1365 |
| ECI1 | enoyl-CoA delta isomerase 1 | HGNC:2703 |
| MMRN2 | multimerin 2 | HGNC:19888 |
| ERV3-1 | endogenous retrovirus group 3, member 1 | HGNC:3454 |
| FMO4 | flavin containing monooxygenase 4 | HGNC:3772 |
| IL36RN | interleukin 36 receptor antagonist | HGNC:15561 |
| IL37 | interleukin 37 | HGNC:15563 |
| KRT6A | keratin 6A | HGNC:6443 |
| CERS4 | ceramide synthase 4 | HGNC:23747 |
| LHFPL3 | lipoma HMGIC fusion partner-like 3 | HGNC:6589 |
| LTBP3 | latent transforming growth factor beta binding protein 3 | HGNC:6716 |
| LTBP2 | latent transforming growth factor beta binding protein 2 | HGNC:6715 |
| LY6G6F | lymphocyte antigen 6 complex, locus G6F | HGNC:13933 |
| MRC1 | mannose receptor, C type 1 | HGNC:7228 |
| ODZ1 | odz, odd Oz/ten-m homolog 1 (Drosophila) | HGNC:8117 |
| SLC50A1 | solute carrier family 50 (sugar transporter), member 1 | HGNC:30657 |
| CRABP1 | cellular retinoic acid binding protein 1 | HGNC:2338 |
| MSMO1 | methylsterol monooxygenase 1 | HGNC:10545 |
| SCGB2B2 | secretoglobin, family 2B, member 2 | HGNC:27616 |
| MSRB1 | methionine sulfoxide reductase B1 | HGNC:14133 |
| SGK3 | serum/glucocorticoid regulated kinase family, member 3 | HGNC:10812 |
| PMEL | premelanosome protein | HGNC:10880 |
| XBP1P1 | X-box binding protein 1 pseudogene 1 | HGNC:12802 |
| SOAT2 | sterol O-acyltransferase 2 | HGNC:11178 |
| CLMP | CXADR-like membrane protein | HGNC:24039 |
| PARD3 | par-3 partitioning defective 3 homolog (C. elegans) | HGNC:16051 |
| PGAP1 | post-GPI attachment to proteins 1 | HGNC:25712 |
| MFRP | membrane frizzled-related protein | HGNC:18121 |
| CXCL10 | chemokine (C—X—C motif) ligand 10 | HGNC:10637 |
| CHAMP1 | chromosome alignment maintaining phosphoprotein 1 | HGNC:20311 |
| CCRL1 | chemokine (C-C motif) receptor-like 1 | HGNC:1611 |
| CNOT6 | CCR4-NOT transcription complex, subunit 6 | HGNC:14099 |
| CCRN4L | CCR4 carbon catabolite repression 4-like (S. cerevisiae) | HGNC:14254 |
| CTLA4 | cytotoxic T-lymphocyte-associated protein 4 | HGNC:2505 |
| CDH19 | cadherin 19, type 2 | HGNC:1758 |
| CDH20 | cadherin 20, type 2 | HGNC:1760 |
| DDX11 | DEAD/H (Asp-Glu-Ala-Asp/His) box helicase 11 | HGNC:2736 |
| RHOV | ras homolog family member V | HGNC:18313 |
| CLCF1 | cardiotrophin-like cytokine factor 1 | HGNC:17412 |
| CPA4 | carboxypeptidase A4 | HGNC:15740 |
| HOMER2 | homer homolog 2 (Drosophila) | HGNC:17513 |
| CSTB | cystatin B (stefin B) | HGNC:2482 |
| VSIG2 | V-set and immunoglobulin domain containing 2 | HGNC:17149 |
| DSC3 | desmocollin 3 | HGNC:3037 |
| DUSP12 | dual specificity phosphatase 12 | HGNC:3067 |
| SH3D19 | SH3 domain containing 19 | HGNC:30418 |
| GLB1 | galactosidase, beta 1 | HGNC:4298 |
| CNTN1 | contactin 1 | HGNC:2171 |
| FADS1 | fatty acid desaturase 1 | HGNC:3574 |
| KHSRP | KH-type splicing regulatory protein | HGNC:6316 |
| HPLH1 | hemophagocytic lymphohistiocytosis 1 | HGNC:23824 |
| CFH | complement factor H | HGNC:4883 |
| SLC25A16 | solute carrier family 25 (mitochondrial carrier; Graves disease | HGNC:10986 |
| | autoantigen), member 16 | |
| PAEP | progestagen-associated endometrial protein | HGNC:8573 |
| GOLT1B | golgi transport 1B | HGNC:20175 |
| GOLT1A | golgi transport 1A | HGNC:24766 |
| DPAGT1 | dolichyl-phosphate (UDP-N-acetylglucosamine) N- | HGNC:2995 |
| | acetylglucosaminephosphotransferase 1 (GlcNAc-1-P transferase) | |
| LGALSL | lectin, galactoside-binding-like | HGNC:25012 |
| KRT123P | keratin 123 pseudogene | HGNC:30196 |
| KRT121P | keratin 121 pseudogene | HGNC:30198 |
| HBB | hemoglobin, beta | HGNC:4827 |
| KCNA5 | potassium voltage-gated channel, shaker-related subfamily, member | HGNC:6224 |
| HOOK2 | hook homolog 2 (Drosophila) | HGNC:19885 |
| KIF2A | kinesin heavy chain member 2A | HGNC:6318 |
| CAPN5 | calpain 5 | HGNC:1482 |
| KLK1 | kallikrein 1 | HGNC:6357 |
| KRTAP5-9 | keratin associated protein 5-9 | HGNC:23604 |
| KRTAP5-5 | keratin associated protein 5-5 | HGNC:23601 |
| KRTAP5-8 | keratin associated protein 5-8 | HGNC:23603 |
| KRTAP5-7 | keratin associated protein 5-7 | HGNC:23602 |
| KRTAP5-11 | keratin associated protein 5-11 | HGNC:23606 |
| KRTAP5-2 | keratin associated protein 5-2 | HGNC:23597 |
| KRTAP5-3 | keratin associated protein 5-3 | HGNC:23598 |
| SPNS1 | spinster homolog 1 (Drosophila) | HGNC:30621 |
| TOX4 | TOX high mobility group box family member 4 | HGNC:20161 |
| C1D | C1D nuclear receptor corepressor | HGNC:29911 |
| PCDHB16 | protocadherin beta 16 | HGNC:14546 |
| MS4A2 | membrane-spanning 4-domains, subfamily A, member 2 | HGNC:7316 |
| MS4A4A | membrane-spanning 4-domains, subfamily A, member 4A | HGNC:13371 |
| NPRL2 | nitrogen permease regulator-like 2 (S. cerevisiae) | HGNC:24969 |
| NELL2 | NEL-like 2 (chicken) | HGNC:7751 |
| MYCBP2 | MYC binding protein 2, E3 ubiquitin protein ligase | HGNC:23386 |
| PODXL | podocalyxin-like | HGNC:9171 |
| PORCN | porcupine homolog (Drosophila) | HGNC:17652 |
| RBL1 | retinoblastoma-like 1 (p107) | HGNC:9893 |
| PVRL4 | poliovirus receptor-related 4 | HGNC:19688 |
| PXDN | peroxidasin homolog (Drosophila) | HGNC:14966 |
| NCOA3 | nuclear receptor coactivator 3 | HGNC:7670 |
| ARID4A | AT rich interactive domain 4A (RBP1-like) | HGNC:9885 |
| POLR2D | polymerase (RNA) II (DNA directed) polypeptide D | HGNC:9191 |
| SEPSECS | Sep (O-phosphoserine) tRNA:Sec (selenocysteine) tRNA synthase | HGNC:30605 |
| SLIT3 | slit homolog 3 (Drosophila) | HGNC:11087 |
| SLIT1 | slit homolog 1 (Drosophila) | HGNC:11085 |
| CXXC1 | CXXC finger protein 1 | HGNC:24343 |
| DDR2 | discoidin domain receptor tyrosine kinase 2 | HGNC:2731 |
| CPAMD8 | C3 and PZP-like, alpha-2-macroglobulin domain | HGNC:23228 |

TABLE 2
Pathway enriched based on the ~2000 up and down regulated genes.
The presentation of enriched pathways based on DAVID database along with the source
database where the pathways are from and have a enrichment calculation
algorithm. We used a conservative cut-off with FDR <= 0.1 to filter out important
pathways. In case, you need a different cut-off, we have also attached the excel
spread-sheet listing all the pathways pass the DAVID cut-off.

| Database | PathwayName | Pvalue | Fold Enrichment | FDR |
|---|---|---|---|---|
| KEGG | Valine, leucine and isoleucine degradation | 4.39E−09 | 4.17 | 5.40E−06 |
| KEGG | PPAR signaling pathway | 6.22E−09 | 3.38 | 7.64E−06 |
| KEGG | Viral myocarditis | 5.98E−08 | 3.17 | 7.35E−05 |
| KEGG | Type I diabetes mellitus | 4.40E−07 | 3.77 | 5.41E−04 |
| KEGG | Allograft rejection | 1.07E−06 | 3.94 | 1.32E−03 |
| KEGG | Systemic lupus erythematosus | 2.41E−06 | 2.53 | 2.96E−03 |
| KEGG | Cell adhesion molecules (CAMs) | 2.89E−06 | 2.27 | 3.55E−03 |
| KEGG | Graft−versus−host disease | 3.91E−06 | 3.63 | 4.80E−03 |
| KEGG | Steroid biosynthesis | 5.01E−06 | 5.39 | 6.15E−03 |
| KEGG | Hematopoietic cell lineage | 1.39E−05 | 2.52 | 1.71E−02 |
| KEGG | ECM−receptor interaction | 2.83E−05 | 2.48 | 3.48E−02 |
| KEGG | Glycolysis/Gluconeogenesis | 4.08E−05 | 2.78 | 5.01E−02 |
| KEGG | Autoimmune thyroid disease | 4.97E−05 | 2.94 | 6.10E−02 |
| REACTOME | Metabolism of lipids and lipoproteins | 2.31E−18 | 3.39 | 2.26E−15 |
| REACTOME | Signaling in Immune system | 1.20E−07 | 1.93 | 1.17E−04 |
| REACTOME | Metabolism of carbohydrates | 7.72E−07 | 2.70 | 7.57E−04 |
| REACTOME | Biological oxidations | 6.38E−05 | 2.19 | 6.25E−02 |
| PANTHER | Cholesterol biosynthesis | 4.79E−05 | 6.43 | 5.46E−02 |
| BIOCARTA | T Cytotoxic Cell Surface Molecules | 2.05E−05 | 5.79 | 2.59E−02 |

TABLE 3
List of gene signatures/regulatory factors controlling the expression of skin aging-
related genes in various pathways and/or gene networks.

| Symbol | Name | HGNC ID |
|---|---|---|
| IL6 | interleukin 6 (interferon, beta 2) | HGNC: 6018 |
| CSF3 | colony stimulating factor 3 (granulocyte) | HGNC: 2438 |
| IFNG | interferon, gamma | HGNC: 5438 |
| IL8 | interleukin 8 | HGNC: 6025 |
| CXCL2 | chemokine (C-X-C motif) ligand 2 | HGNC: 4603 |
| CXCL9 | chemokine (C-X-C motif) ligand 9 | HGNC: 7098 |
| CCKAR | cholecystokinin A receptor | HGNC: 1570 |
| CSN1S1 | casein alpha 1 | HGNC: 2445 |
| CXCL10 | chemokine (C-X-C motif) ligand 10 | HGNC: 10637 |
| CXCL1 | chemokine (C-X-C motif) ligand 1 (melanoma growth | HGNC: 4602 |
| | stimulating activity, alpha) | |
| FOSB | FBJ murine osteosarcoma viral oncogene homolog B | HGNC: 3797 |
| DEFA6 | defensin, alpha 6, Paneth cell-specific | HGNC: 2765 |
| NR4A2 | nuclear receptor subfamily 4, group A, member 2 | HGNC: 7981 |
| EGFL6 | EGF-like-domain, multiple 6 | HGNC: 3235 |
| NR4A1 | nuclear receptor subfamily 4, group A, member 1 | HGNC: 7980 |
| CD38 | CD38 molecule | HGNC: 1667 |
| SOCS3 | suppressor of cytokine signaling 3 | HGNC: 19391 |
| RGS1 | regulator of G-protein signaling 1 | HGNC: 9991 |
| FOSL1 | FOS-like antigen 1 | HGNC: 13718 |
| SAA4 | serum amyloid A4, constitutive | HGNC: 10516 |
| PCK1 | phosphoenolpyruvate carboxykinase 1 (soluble) | HGNC: 8724 |
| LIPE | lipase, hormone-sensitive | HGNC: 6621 |
| MMP1 | matrix metallopeptidase 1 (interstitial collagenase) | HGNC: 7155 |
| LEP | leptin | HGNC: 6553 |
| BPIL1 | bactericidal/permeability-increasing protein-like 1 | HGNC: 16177 |
| TUSC5 | tumor suppressor candidate 5 | HGNC: 29592 |
| FIGF | e-fos induced growth factor (vascular endothelial growth factor | HGNC: 3708 |
| | D) | |
| GYS2 | glycogen synthase 2 (liver) | HGNC: 4707 |
| LHFPL3 | lipoma HMGIC fusion partner-like 3 | HGNC: 6589 |
| CXCL11 | chemokine (C-X-C motif) ligand 11 | HGNC: 10638 |
| ARHGDIG | Rho GDP dissociation inhibitor (GDI) gamma | HGNC: 680 |
| CCL4L2 | chemokine (C-C motif) ligand 4-like 2 | HGNC: 24066 |
| ACVRIC | activin A receptor, type IC | HGNC: 18123 |
| NR4A3 | nuclear receptor subfamily 4, group A, member 3 | HGNC: 7982 |
| PLIN1 | perilipin 1 | HGNC: 9076 |
| OSM | oncostatin M | HGNC: 8506 |
| FABP12 | fatty acid binding protein 12 | HGNC: 34524 |
| SAA1 | serum amyloid A1 | HGNC: 10513 |
| FABP4 | fatty acid binding protein 4, adipocyte | HGNC: 3559 |
| IL20 | interleukin 20 | HGNC: 6002 |
| GZMB | granzyme B (granzyme 2, cytotoxic T-lymphocyte-associated | HGNC: 4709 |
| | serine esterase 1) | |
| CD69 | CD69 molecule | HGNC: 1694 |
| SAA2 | serum amyloid A2 | HGNC: 10514 |
| ATF3 | activating transcription factor 3 | HGNC: 785 |
| PFKFB1 | 6-phosphofructo-2-kinase/fructose-2,6-biphosphatase 1 | HGNC: 8872 |
| CYR61 | cysteine-rich, angiogenic inducer, 61 | HGNC: 2654 |
| JAKMIP1 | janus kinase and microtubule interacting protein 1 | HGNC: 26460 |
| PKLR1 | pyruvate kinase, liver and RBC | HGNC: 9020 |
| ALDOB | aldolase B, fructose-biphosphate | HGNC: 417 |
| LDHC | lactate dehydrogenase C | HGNC: 6544 |
| HSD3B2 | hydroxy-delta-5-steroid dehydrogenase, 3 beta- and steroid | HGNC: 5218 |
| | delta-isomerase 2 | |
| HSD3B1 | hydroxy-delta-5-steroid dehydrogenase, 3 beta- and steroid | HGNC: 3283 |
| | delta-isomerase 1 | |
| PDZK1 | PDZ domain containing 1 | HGNC: 5174 |
| SLC26A3 | solute carrier family 26, member 3 | HGNC: 1811 |
| FABP6 | atty acid binding protein 6, ileal | HGNC: 2172 |
| FABP7 | fatty acid binding protein 7, brain | HGNC: 2173 |
| FADS1 | fatty acid desaturase 1 | HGNC: 3992 |
| FADS2 | fatty acid desaturase 2 | HGNC: 9415 |
| CNDP1 | carnosine dipeptidase 1 (metallopeptidase M20 family) | HGNC: 84735 |
| UBP1 | Ureidopropionase, beta (CN hydrolase family) | HGNC: 51733 |
| ELOVL3 | Elongation of very long chain fatty acids | HGNC: 83401 |
| TREH | trehalase (brush-border membrane glycoprotein) | HGNC: 11181 |
| AMY1A | Amylase, alpha 1A (salivary) | HGNC: 276 |
| MUC7 | mucin 7, secreted | HGNC: 4589 |
| APOC1 | apolipoprotein C-1 | HGNC: 341 |
| ACSBG1 | acyl-CoA synthatase bubblegum family member 1 | HGNC: 23205 |
| MUC1 | mucin 1, cell surface associated | HGNC: 4582 |
| GSK3B | glycogen synthase kinase 3 beta | HGNC: 2932 |
| EGFR | epidermal growth factor receptor | HGNC: 1956 |
| ESR1 | estrogen receptor 1 | HGNC: 2099 |
| ABL1 | c-abl ocongene 1, non-receptor tyrosine kinase | HGNC: 25 |
| CTNNB1 | catenin (cadherin-associated protein), beta 1, 88 kDa | HGNC: 1499 |
| GAGE2C G | antigen 2C | HGNC: 2574 |
| OR52E8 | alfactory receptor, family 52, subfamily E, member 8 | HGNC: 390079 |
| MAPK1 | mitogen-activated protein kinase 1 | HGNC: 5594 |
| STAT3 | signal transducer and activator of transcription 3 (acute-phase | HGNC: 6774 |
| | response factor) | |
| NFKB1 | nuclear factor of kappa light polypeptide gene enhancer in B- | HGNC: 4790 |
| | cells 1 | |
| JUN | an proto-oncogene | HGNC: 3725 |

HGNC: HUGO Gene Nomenclature Committee url: http://www.genenames.org/
Note
that the gene described in italics at the end of the table are not part of Table 1 (it only includes the top 2000 up and down regulated genes); these italicized genes are part of the gene network regulators.

TABLE 4
up- and down- regulation of genes

| Gene ID | log2FoldChange |
|---|---|
| A2ML1 | 5.232038252 |
| AACS | 4.080550359 |
| AADACL3 | 3.725566743 |
| AADACL4 | 3.705855774 |
| ABCA13 | 3.33791816 |
| ABCA4 | 3.202673123 |
| ABCA6 | 2.961209119 |
| ABCA8 | 2.812579577 |
| ABCA9 | 2.760008088 |
| ABCB5 | 2.568469326 |
| ABCC11 | 2.501770187 |
| ABCC4 | 2.413636014 |
| ABHD12B | 2.383847689 |
| ABHD3 | 2.288932795 |
| ABHD5 | 2.253846967 |
| ABLIM2 | 2.214099057 |
| ABLIM3 | 2.167263213 |
| ACAA1 | 2.097514835 |
| ACAA2 | 2.089701582 |
| ACAD8 | 2.084597545 |
| ACADM | 2.019198644 |
| ACADVL | 1.980871868 |
| ACAP1 | 1.902109447 |
| ACAT2 | 1.852622907 |
| ACE | 1.852383074 |
| ACE2 | 1.848437824 |
| ACLY | 1.843490132 |
| ACO1 | 1.83798896 |
| ACOT1 | 1.828371596 |
| ACOT2 | 1.806970784 |
| ACOT4 | 1.799871164 |
| ACOT7 | 1.78933249 |
| ACOX2 | 1.776943169 |
| ACP6 | 1.765634945 |
| ACPP | 1.761788468 |
| ACSBGI | 1.761560397 |
| ACSL1 | 1.760241809 |
| ACSL3 | 1.757608676 |
| ACSLS | 1.741815779 |
| ACSM3 | 1.741500471 |
| ACSM5 | 1.737979248 |
| ACSS2 | 1.733440435 |
| ACTA1 | 1.731120615 |
| ACTA2 | 1.691612489 |
| ACTBL2 | 1.686446343 |
| ACTC1 | 1.673273334 |
| ACTG2 | 1.672073089 |
| ACTN1 | 1.659898906 |
| ACTR6 | 1.655515583 |
| ACVR1C | 1.634133301 |
| ACVRL1 | 1.620276681 |
| ACY1 | 1.610307087 |
| ADAM33 | 1.58406277 |
| ADAM8 | 1.575793381 |
| ADAMTS15 | 1.574263825 |
| ADAMTS5 | 1.574078143 |
| ADAMTS7 | 1.550685556 |
| ADAMTSL1 | 1.549161629 |
| ADAMTSL3 | 1.547103771 |
| ADAMTSLS | 1.529584451 |
| ADCK1 | 1.508070416 |
| ADCY5 | 1.500080295 |
| ADH1A | 1.488934892 |
| ADH1B | 1.479869122 |
| ADH1C | 1.476996607 |
| ADH6 | 1.467829493 |
| ADHFL1 | 1.460316269 |
| ADIPOQ | 1.441750612 |
| ADRA2A | 1.434237726 |
| AEBP1 | 1.433240506 |
| AGPAT1 | 1.427009839 |
| AGPAT3 | 1.426814356 |
| AGPHD1 | 1.423607062 |
| AGR2 | 1.422523795 |
| AGRN | 1.421273275 |
| AGTRAP | 1.418689497 |
| AHSP | 1.416074982 |
| AKAP12 | 1.412103606 |
| AKAP2 | 1.408745781 |
| AKNA | 1.405734349 |
| AKR1A1 | 1.405058571 |
| ALAS1 | 1.391295285 |
| ALAS2 | 1.387922866 |
| ALCAM | 1.375534132 |
| ALDH1B1 | 1.365090643 |
| ALDH1L1 | 1.355041255 |
| ALDH3A1 | 1.354717154 |
| ALDH3B2 | 1.347098017 |
| ALDH6A1 | 1.334078453 |
| ALDOB | 1.325953664 |
| ALDOC | 1.314987024 |
| ALG1L | 1.303849161 |
| ALOX15B | 1.300565313 |
| ALPK2 | 1.290316723 |
| AMACR | 1.285915698 |
| AMICAI | 1.283991875 |
| AMOT | 1.283895871 |
| AMOTL2 | 1.282276837 |
| AMPD1 | 1.263993007 |
| AMTN | 1.259721834 |
| AMY1A | 1.254402487 |
| AMY1B | 1.253325064 |
| AMY1C | 1.248710176 |
| ANGPTI | 1.239085271 |
| ANGPTL2 | 1.236238143 |
| ANGPTL4 | 1.234793741 |
| ANGPTL5 | 1.219705692 |
| ANGPTL7 | 1.219381934 |
| ANKRD1 | 1.211951612 |
| ANKRD22 | 1.204764559 |
| ANKS6 | 1.203042217 |
| ANPEP | 1.201433379 |
| ANTXR2 | 1.200115748 |
| ANXA3 | 1.197427942 |
| ANXA6 | 1.197184453 |
| AOC3 | 1.189948198 |
| AOX1 | 1.188424407 |
| APBB1IP | 1.184874211 |
| APOBEC3G | 1.18093333 |
| APOC1 | 1.175109861 |
| APOD | 1.167265269 |
| APOE | 1.165869476 |
| APOL1 | 1.163700968 |
| APOL3 | 1.163133343 |
| APOL6 | 1.159161307 |
| AQP4 | 1.154896716 |
| AQP5 | 1.151012768 |
| AQP7 | 1.144851374 |
| AREG | 1.144230495 |
| AREGB | 1.142744141 |
| ARHGAP20 | 1.135201358 |
| ARHGAP29 | 1.126299843 |
| ARHGAP30 | 1.125005561 |
| ARHGAP6 | 1.118435563 |
| ARHGDIB | 1.116034827 |
| ARHGEF6 | 1.10244655 |
| ARL6IP1 | 1.098174751 |
| ARR3 | 1.097858046 |
| ARSH | 1.094469072 |
| ASAM | 1.091336285 |
| ASIP | 1.076112291 |
| ASTN1 | 1.072358931 |
| ATOH1 | 1.071959393 |
| ATOH8 | 1.06171746 |
| ATP12A | 1.055952873 |
| ATP1A2 | 1.054836184 |
| ATP5C1 | 1.052303724 |
| ATP5G3 | 1.048297241 |
| ATP5I | 1.048075539 |
| ATP6VOA4 | 1.044224499 |
| ATP6VOB | 1.042247045 |
| ATP6VOC | 1.040727114 |
| ATP6V1C2 | 1.039340118 |
| AVIL | 1.038198856 |
| AWAT1 | 1.035847539 |
| AWAT2 | 1.027746543 |
| B3GNT3 | 1.026300251 |
| B4GALNT2 | 1.021337432 |
| BAH | 1.017206262 |
| BARX2 | 1.016925237 |
| BCAN | 1.012246448 |
| BCAP29 | 1.011200565 |
| BCAP31 | 1.0103578 |
| BCAT1 | 1.004877622 |
| BCAT2 | 1.002735372 |
| BCKDHB | 0.998635214 |
| BDH1 | 0.996523706 |
| BEAN1 | 0.99308757 |
| BHMT2 | 0.990062406 |
| BICC1 | 0.984397677 |
| BIRC3 | 0.976297686 |
| BLNK | 0.975649143 |
| BRI3BP | 0.969430675 |
| BRP44 | 0.96917848 |
| BRP44L | 0.961102823 |
| BSG | 0.960242752 |
| BST1 | 0.959113336 |
| BST2 | 0.958044351 |
| BTN3A1 | 0.950424103 |
| BTN3A2 | 0.947981912 |
| BTN3A3 | 0.943516128 |
| BZW1 | 0.942613394 |
| Cl0orf129 | 0.940311238 |
| Cl0orf57 | 0.931744078 |
| C10orf58 | 0.931389357 |
| C11orf96 | 0.930836216 |
| C12orf39 | 0.929331896 |
| C12orf64 | 0.927058692 |
| C13orf36 | 0.920042594 |
| Cl4orf180 | 0.914186227 |
| C14orf2 | 0.91385926 |
| C14orf49 | 0.904358065 |
| C15orf23 | 0.90410343 |
| C15orf43 | 0.902489928 |
| C15orf48 | 0.899742683 |
| Cl6orf73 | 0.897654572 |
| C16orf74 | 0.897319749 |
| C16orf89 | 0.891418909 |
| C17orf97 | 0.88725701 |
| C19orf28 | 0.884549425 |
| C19orf46 | 0.883918413 |
| C19orf71 | 0.88287833 |
| Clorf104 | 0.877772162 |
| Clorf113 | 0.876792387 |
| Clorf122 | 0.869490471 |
| Clorf125 | 0.868635503 |
| C1orf158 | 0.866593789 |
| Clorf162 | 0.856422456 |
| Clorf5l | 0.85361664 |
| Clorf53 | 0.851849469 |
| Clorf68 | 0.850683059 |
| Clorf9 | 0.848017236 |
| C1QA | 0.842038447 |
| C1QB | 0.841688189 |
| C1QC | 0.840617385 |
| C1QTNF5 | 0.83930445 |
| C1QTNF6 | 0.835742329 |
| C1R | 0.835366478 |
| C1S | 0.82925104 |
| C2 | 0.828081804 |
| C20orf103 | 0.827752937 |
| C20orf24 | 0.82673847 |
| C20orf3 | 0.825665684 |
| C21orf33 | 0.825016444 |
| C3 | 0.82319498 |
| C3AR1 | 0.821201653 |
| C4orf23 | 0.813087813 |
| C4orf31 | 0.812533891 |
| C5orf4 | 0.812098722 |
| C6orf105 | 0.811577891 |
| C6orf106 | 0.808618456 |
| C6orf126 | 0.807958993 |
| C6orf26 | 0.807917972 |
| C6orf94 | 0.806442564 |
| C7 | 0.801725925 |
| C7orf58 | 0.800896147 |
| C7orf68 | 0.798953825 |
| CCR4 | 0.748940725 |
| C9orf16 | 0.795551973 |
| C9orf7 | 0.793758218 |
| CA14 | 0.793468014 |
| CA6 | 0.792626914 |
| CALCB | 0.791137389 |
| CALD1 | 0.786509848 |
| CALML3 | 0.78380644 |
| CAMK1G | 0.782937966 |
| CAMP | 0.780273373 |
| CAPN13 | 0.77814649 |
| CAPN3 | 0.77638503 |
| CAPN8 | 0.775634239 |
| CARD11 | 0.772918944 |
| CARD18 | 0.770869921 |
| CASQ2 | 0.770769276 |
| CASS4 | 0.770620159 |
| CBLN1 | 0.765830306 |
| CBLN2 | 0.765746887 |
| CCBP2 | 0.764914593 |
| CCDC69 | 0.764870382 |
| CCDC85A | 0.763278452 |
| CCDC86 | 0.762945653 |
| CCK | 0.762709079 |
| CCKBR | 0.76267341 |
| CCL13 | 0.762218024 |
| CCL14 | 0.76179936 |
| CCL15 | 0.761409502 |
| CCL17 | 0.76094414 |
| CCL18 | 0.758899798 |
| CCL19 | 0.757671358 |
| CCL27 | 0.755513617 |
| CCL4 | 0.753719824 |
| CCL4L2 | 0.753256985 |
| CCL5 | 0.75171897 |
| CCNC | 0.751032062 |
| CCND1 | 0.75100013 |
| CCNF | 0.750948895 |
| CCR1 | 0.749275262 |
| CCR2 | 0.74901499 |
| C9orf140 | 0.797269786 |
| CCR5 | 0.748840622 |
| CCR7 | 0.747443487 |
| CD163 | 0.746895014 |
| CD1C | 0.745484678 |
| CD1E | 0.744287962 |
| CD2 | 0.744059612 |
| CD207 | 0.743132085 |
| CD209 | 0.739038868 |
| CD226 | 0.736591855 |
| CD24 | 0.736216427 |
| CD247 | 0.735954286 |
| CD248 | 0.734122479 |
| CD27 | 0.733476618 |
| CD28 | 0.733425584 |
| CD34 | 0.733371965 |
| CD36 | 0.733005039 |
| CD37 | 0.731592379 |
| CD38 | 0.730346031 |
| CD3D | 0.730323379 |
| CD3E | 0.728577927 |
| CD4OLG | 0.725829232 |
| CD48 | 0.723778399 |
| CD5 | 0.723184088 |
| CD52 | 0.72168319 |
| CD53 | 0.718476028 |
| CD6 | 0.718229421 |
| CD68 | 0.717801714 |
| CD74 | 0.717400398 |
| CD84 | 0.717372863 |
| CD8A | 0.717216953 |
| CD96 | 0.71571822 |
| CD97 | 0.712842251 |
| CDA | 0.710635861 |
| CDH5 | 0.709367024 |
| CDH7 | 0.708961348 |
| CDK5 | 0.708420828 |
| CDKL2 | 0.708341515 |
| CDKN1A | 0.708063289 |
| CDO1 | 0.707175314 |
| CEACAM1 | 0.705976589 |
| CEACAM6 | 0.704684521 |
| CECR2 | 0.70067378 |
| CELF2 | 0.700594894 |
| CES1 | 0.699640956 |
| CFB | 0.699438037 |
| CFD | 0.697056958 |
| CFH | 0.692238866 |
| CFHR1 | 0.691912767 |
| CFHR3 | 0.691771062 |
| CGA | 0.688193587 |
| CHCHD10 | 0.686823982 |
| CHGA | 0.686256791 |
| CHGB | 0.6840037 |
| CHI3L1 | 0.683256591 |
| CHI3L2 | 0.681823307 |
| CHIT1 | 0.681679494 |
| CHL1 | 0.681431466 |
| CHP | 0.67660651 |
| CHRDLI | 0.675579307 |
| CHRDL2 | 0.67553655 |
| CHRNA9 | 0.675076329 |
| CHST2 | 0.674405775 |
| CIDEA | 0.674304617 |
| CIDEC | 0.673569412 |
| CILP | 0.673553398 |
| CILP2 | 0.673102173 |
| CISH | 0.67068239 |
| CKB | 0.66864368 |
| CKMT1A | 0.668581748 |
| CKMT1B | 0.667304308 |
| CLC | 0.667280993 |
| CLCA2 | 0.666510809 |
| CLDN10 | 0.663957279 |
| CLDN11 | 0.662363671 |
| CLDN5 | 0.661150212 |
| CLEC10A | 0.660670207 |
| CLEC12A | 0.66019268 |
| CLEC2A | 0.659533948 |
| CLEC3B | 0.659181375 |
| CLEC4G | 0.657807469 |
| CLIC2 | 0.656902749 |
| CLIC3 | 0.654887307 |
| CLIC6 | 0.65439818 |
| CLIP2 | 0.653848693 |
| CLIP3 | 0.653639962 |
| CLSTN3 | 0.652488207 |
| CLU | 0.651219801 |
| CMA1 | 0.648360441 |
| CMTM8 | 0.647054101 |
| CNDP1 | 0.646608556 |
| CNDP2 | 0.646165968 |
| CNIH4 | 0.64556632 |
| CNKSR3 | 0.642491771 |
| CNN1 | 0.640289415 |
| CNPY1 | 0.637826241 |
| COCH | 0.637181915 |
| COL11A1 | 0.636839925 |
| COL12A1 | 0.635940089 |
| COL16A1 | 0.635281955 |
| COL1Al | 0.633662888 |
| COL1A2 | 0.632689827 |
| COL3A1 | 0.631890797 |
| COL4A1 | 0.631579102 |
| COL4A2 | 0.629278808 |
| COL4A6 | 0.628472358 |
| COL5A1 | 0.626403674 |
| COL7A1 | 0.622171021 |
| COL5A1 | 0.621627606 |
| COMP | 0.6213615 |
| COQ4 | 0.620432773 |
| COQ9 | 0.620139929 |
| CORO1A | 0.618806539 |
| COTL1 | 0.617518298 |
| COX5A | 0.617426889 |
| COX6C | 0.616145976 |
| COX7A2 | 0.615282691 |
| COX7B | 0.613751991 |
| COX7C | 0.612974235 |
| COX8A | 0.611463404 |
| CP | 0.610400505 |
| CPA3 | 0.608812298 |
| CPA4 | 0.608183636 |
| CPB2 | 0.607393505 |
| CPD | 0.607164577 |
| CPE | 0.6071146 |
| CPEB4 | 0.606576095 |
| CPLX3 | 0.604875519 |
| CPT2 | 0.6046887 |
| CPVL | 0.604269631 |
| CPXM1 | 0.602189623 |
| CPZ | 0.600586734 |
| CRABP1 | 0.597909379 |
| CRAT | 0.596720556 |
| CREG1 | 0.595727948 |
| CRISP3 | 0.59528462 |
| CRYAB | 0.594419199 |
| CRYBB2 | 0.593911532 |
| CS | 0.592384522 |
| CSAG3 | 0.591530447 |
| CSF1 | 0.591527932 |
| CSF1R | 0.591023422 |
| CSN1S1 | 0.590746098 |
| CSPG4 | 0.590717207 |
| CSRP2 | 0.58959508 |
| CST1 | 0.588973383 |
| CST6 | 0.588296267 |
| CST7 | 0.588167494 |
| CSTB | 0.587950682 |
| CTAG2 | 0.587238503 |
| CTH | 0.586221894 |
| CTHRCI | 0.586151017 |
| CTNND2 | 0.585631706 |
| CTSG | 0.58554417 |
| CTSK | 0.585534422 |
| CTSL2 | 0.584352618 |
| CTSS | 0.583898886 |
| CUBN | 0.583407172 |
| CUX2 | 0.582747052 |
| CXCL10 | 0.582537857 |
| CXCL11 | 0.581158017 |
| CXCL9 | 0.580570674 |
| CXCR3 | 0.580561381 |
| CXCR7 | 0.580068749 |
| CYB5A | 0.579131054 |
| CYBB | 0.577935074 |
| CYBRD1 | 0.576732674 |
| CYCS | 0.575502733 |
| CYP19A1 | 0.57529074 |
| CYP1A1 | 0.57340909 |
| CYP1A2 | 0.572169566 |
| CYP1B1 | 0.571600726 |
| CYP26B1 | 0.567724157 |
| CYP27A1 | 0.567494882 |
| CYP27B1 | 0.567192991 |
| CYP2R1 | 0.564830904 |
| CYP3A4 | 0.563584501 |
| CYP4B1 | 0.560296992 |
| CYP4F2 | 0.558822441 |
| CYP4F22 | 0.558064138 |
| CYP4F3 | 0.557536701 |
| CYP4F8 | 0.557400907 |
| CYP51A1 | 0.556847797 |
| CYTH4 | 0.556541709 |
| DACH1 | 0.556495194 |
| DAPK2 | 0.55643176 |
| DBT | 0.556294248 |
| DCD | 0.555938734 |
| DCI | 0.555202823 |
| DCT | 0.554212398 |
| DCXR | 0.553250615 |
| DDC | 0.553206096 |
| DDHD1 | 0.552844184 |
| DDIT4 | 0.551724084 |
| DDO | 0.551158736 |
| DDR2 | 0.550486387 |
| DDX43 | 0.549939754 |
| DDX60 | 0.549534157 |
| DEFA6 | 0.548742699 |
| DEFB132 | 0.547219123 |
| DENND3 | 0.547156719 |
| DES | 0.546995716 |
| DGAT2 | 0.546570087 |
| DGAT2L6 | 0.545608075 |
| DHCR24 | 0.544674381 |
| DHCR7 | 0.544444263 |
| DHRS11 | 0.54360883 |
| DHRS2 | 0.540802501 |
| DHRS7B | 0.540744778 |
| DHRS9 | 0.540429524 |
| D102 | 0.539530158 |
| DIRAS 1 | 0.539329212 |
| DIRAS3 | 0.539258665 |
| DKK1 | 0.538978494 |
| DLD | 0.538656747 |
| DLK1 | 0.53703431 |
| DMBX1 | 0.535852296 |
| DMRTA1 | 0.534608355 |
| DNAH17 | 0.532919317 |
| DNAH8 | 0.532281963 |
| DNASEIL2 | 0.531698368 |
| DOC2B | 0.529777062 |
| DOCK11 | 0.527318535 |
| DOCK2 | 0.52575 |
| DOK2 | 0.5251 |
| DOPEY2 | 0.524154558 |
| DPEP1 | 0.524067295 |
| DPP4 | 0.523670923 |
| DPT | 0.523544437 |
| DPYD | 0.523378009 |
| DRAMI | 0.522613629 |
| DSC2 | 0.522349227 |
| DSE | 0.522319047 |
| DSG4 | 0.521926513 |
| DTNA | 0.521581818 |
| DTX4 | 0.521179582 |
| DUSP1 | 0.520864957 |
| DUSP16 | 0.519933255 |
| DUSP4 | 0.517909682 |
| DUSP5 | 0.517848658 |
| DUSP6 | 0.517792586 |
| EBF1 | 0.517730154 |
| EBI3 | 0.517509195 |
| EBP | 0.516349706 |
| ECH1 | 0.51518304 |
| ECHDC1 | 0.514841211 |
| ECHDC2 | 0.514547832 |
| ECM1 | 0.514458624 |
| ECM2 | 0.514343092 |
| EDA2R | 0.513861922 |
| EDDM3B | 0.513723775 |
| EFCAB4A | 0.513375304 |
| EFEMP1 | 0.513258467 |
| EFNB2 | 0.512917839 |
| EGFL6 | 0.512838555 |
| EGLN3 | 0.51225525 |
| EGR1 | 0.51206884 |
| EGR2 | 0.511003459 |
| EGR3 | 0.510778993 |
| EHD2 | 0.510692469 |
| EHF | 0.510492657 |
| EI24 | 0.510489591 |
| ELANE | 0.510006127 |
| ELAVL2 | 0.5093179 |
| ELL2 | 0.50817644 |
| ELOVL1 | 0.507689446 |
| ELOVL3 | 0.507599462 |
| ELOVL4 | 0.507260864 |
| ELOVL5 | 0.506248238 |
| EMID2 | 0.505320796 |
| EMILIN1 | 0.504268622 |
| EMILIN2 | 0.503644696 |
| EMILIN3 | 0.50263856 |
| EML6 | 0.501499895 |
| EN2 | 0.500916685 |
| ENC1 | 0.500012065 |
| ENDOU | 0.499665741 |
| ENG | 0.497713985 |
| ENO2 | 0.496499653 |
| EOMES | 0.495395643 |
| EPB41L2 | 0.494925743 |
| EPPK1 | 0.49484724 |
| ERAP1 | 0.494808452 |
| ERV3 | 0.493329551 |
| ESRRA | 0.492028503 |
| ETFDH | 0.490659237 |
| ETHE1 | 0.490296755 |
| ETV4 | 0.490270215 |
| EVI2B | 0.489841762 |
| EYA4 | 0.489352923 |
| F10 | 0.488597264 |
| F13A1 | 0.487259535 |
| F3 | 0.48720793 |
| F9 | 0.48673417 |
| FA2H | 0.486577314 |
| FABP12 | 0.486303594 |
| FABP4 | 0.485898265 |
| FABP6 | 0.485222561 |
| FABP7 | 0.48519999 |
| FABP9 | 0.485179665 |
| FADS1 | 0.484857177 |
| FADS2 | 0.481183487 |
| FADS6 | 0.480742796 |
| FAM108C | 10.479530823 |
| FAM126A | 0.479034379 |
| FAM129A | 0.478551739 |
| FAM132A | 0.477436416 |
| FAM150B | 0.477095654 |
| FAM176A | 0.476338419 |
| FAM181A | 0.474180788 |
| FAM196B | 0.473964863 |
| FAM19A2 | 0.472973712 |
| FAM26D | 0.47129739 |
| FAM26E | 0.470000202 |
| FAM3C | 0.469482303 |
| FAM3D | 0.46928969 |
| FAM46C | 0.468636377 |
| FAM69C | 0.467246808 |
| FAM70A | 0.466978034 |
| FAM73B | 0.466642696 |
| FAM75D1 | 0.463984346 |
| FAM78A | 0.463488642 |
| FAM82A2 | 0.463327022 |
| FAM83A | 0.463322813 |
| FAM84A | 0.463074926 |
| FAM84B | 0.463025792 |
| FAR2 | 0.462953519 |
| FASN | 0.461857874 |
| FAS TKD1 | 0.461642503 |
| FBLN2 | 0.461224657 |
| FBLNS | 0.461090095 |
| FBN1 | 0.460820724 |
| FBP 1 | 0.46071627 |
| FBP2 | 0.46043014 |
| FBXO9 | 0.460072274 |
| FCER2 | 0.458826422 |
| FCGBP | 0.4578527 |
| FCGBP | 0.4578527 |
| FCGR2A | 0.45729655 |
| FCGR2B | 0.456578608 |
| FCGR3A | 0.45471022 |
| FCGRT | 0.454024904 |
| FCN1 | 0.452955612 |
| FDFT1 | 0.452776108 |
| FDPS | 0.452041731 |
| FLN1 | 0.451447394 |
| FERMT2 | 0.45079544 |
| EERMT3 | 0.450095285 |
| FETUB | 0.447961211 |
| FGF16 | 0.447692602 |
| FGF18 | 0.446672685 |
| FGF9 | 0.446560585 |
| FGFR1 | 0.446465742 |
| FGFR2 | 0.446452184 |
| FHL1 | 0.446384972 |
| FIBIN | 0.44597033 |
| FIGF | 0.444818416 |
| FTLIP1 | 0.444473374 |
| FITM2 | 0.443436285 |
| FLNA | 0.443148116 |
| FLNC | 0.442931256 |
| FLRT2 | 0.442875917 |
| FLT4 | 0.442724546 |
| FMNL1 | 0.441949503 |
| FMO2 | 0.439383581 |
| FMO3 | 0.438446633 |
| FMOD | 0.437783118 |
| FOS | 0.437656262 |
| FOSB | 0.436782135 |
| FOSL1 | 0.435456258 |
| FOXCl | 0.435229184 |
| FOXQ1 | 0.435068041 |
| FPR2 | 0.434717067 |
| FPR3 | 0.433481626 |
| FREM1 | 0.432798735 |
| FRZB | 0.432584717 |
| FSTL3 | 0.432445979 |
| FTL | 0.430814667 |
| FUT3 | 0.428448779 |
| FXYD5 | 0.427778053 |
| FYN | 0.426143223 |
| FZD4 | 0.42596125 |
| FZD5 | 0.42562054 |
| FZD7 | 0.423642522 |
| GOS2 | 0.422234972 |
| G6PD | 0.421332339 |
| GABBR2 | 0.420294908 |
| GABRA4 | 0.419568551 |
| GABRP | 0.41913361 |
| GADD45G | 0.419100444 |
| GAGE12D | 0.418585219 |
| GAGE12G | 0.418092899 |
| GAGE12I | 0.418026024 |
| GAGE2B | 0.417476314 |
| GAGE2C | 0.416885549 |
| GAGE2D | 0.416232315 |
| GAL | 0.41616708 |
| GALNTL1 | 0.415241439 |
| GALNTL2 | 0.414588797 |
| GALNTL5 | 0.413987457 |
| GALNTL6 | 0.413809399 |
| GAP43 | 0.413082067 |
| GAS1 | 0.412393081 |
| GATA2 | 0.411699719 |
| GATA6 | 0.409777155 |
| GBP3 | 0.409161485 |
| GBP4 | 0.40823574 |
| GBP6 | 0.406675827 |
| GCLM | 0.406199678 |
| GCNT4 | 0.405426509 |
| GCSH | 0.405328259 |
| GDA | 0.402905319 |
| GDF10 | 0.401440696 |
| GDPD2 | 0.400792296 |
| GFM2 | 0.399650739 |
| GFRA1 | 0.399277856 |
| GGPS 1 | 0.398900983 |
| GGT6 | 0.397232289 |
| GIMAP4 | 0.397046987 |
| GJA3 | 0.396442895 |
| GJB2 | 0.396112709 |
| GJB6 | 0.395989602 |
| GJC2 | 0.394744395 |
| GK | 0.393439469 |
| GK5 | 0.393287609 |
| GLDC | 0.393176377 |
| GLIPR1 | 0.392739575 |
| GLRX | 0.392298676 |
| GLRX5 | 0.391956567 |
| GLYAT | 0.391790273 |
| GLYATL1 | 0.390655024 |
| GLYATL1P4 | 0.389167164 |
| GNLY | 0.389113939 |
| GNPAT | 0.388519224 |
| GOT1 | 0.387970871 |
| GPAM | 0.387509463 |
| GPBAR1 | 0.387015245 |
| GPC3 | 0.386701956 |
| GPER | 0.384885424 |
| GPIHBP1 | 0.384362718 |
| GPR124 | 0.383796053 |
| GPR133 | 0.383202367 |
| GPR143 | 0.381926676 |
| GPR15 | 0.381908315 |
| GPR174 | 0.381810225 |
| GPR18 | 0.381390002 |
| GPR182 | 0.380192444 |
| GPR64 | 0.380024147 |
| GPR98 | 0.379606532 |
| GPRC5D | 0.378717053 |
| GPRIN2 | 0.378016339 |
| GPSM3 | 0.376224592 |
| GPT | 0.375757779 |
| GPT2 | 0.375349942 |
| GRAP2 | 0.375167333 |
| GREB1 | 0.374347763 |
| GREM1 | 0.374001977 |
| GRIN2A | 0.373897381 |
| GRIN3A | 0.372221995 |
| GRK5 | 0.371629768 |
| GRM3 | 0.371205788 |
| GRM7 | 0.370099769 |
| GRP | 0.368889749 |
| GSDMA | 0.368563892 |
| GTSF1 | 0.368482596 |
| GUCY2C | 0.367768843 |
| GYG2 | 0.367257476 |
| GYPC | 0.367155053 |
| GYS2 | 0.366803828 |
| GZMA | 0.366594994 |
| GZMB | 0.365585394 |
| GZMH | 0.365326339 |
| GZMK | 0.364726135 |
| GZMM | 0.364148823 |
| HACL1 | 0.363141825 |
| HADH | 0.362621929 |
| HAO2 | 0.362609081 |
| HAPLNI | 0.362562741 |
| HBA1 | 0.362532603 |
| HBA2 | 0.361731395 |
| HBB | 0.360550859 |
| HBD | 0.360135475 |
| HBEGF | 0.360107409 |
| HBG2 | 0.360077239 |
| HCLS1 | 0.360000847 |
| HEATR4 | 0.359508493 |
| HEPACAM | 0.358167932 |
| HEPACAM2 | 0.357383769 |
| HEPHL1 | 0.35679072 |
| HEPN1 | 0.355941294 |
| HGD | 0.35563389 |
| HHATL | 0.355423622 |
| HIBCH | 0.35411249 |
| HIC1 | 0.353743897 |
| HINT1 | 0.353718189 |
| HIP1 | 0.353451166 |
| HIST1H1C | 0.353159511 |
| HIST1H1D | 0.352543122 |
| HIST1H2AE | 0.352088356 |
| HIST1H2BC | 0.351533058 |
| HIST1H2BK | 0.351383587 |
| HIST3H2A | 0.351274488 |
| HK2 | 0.349778086 |
| HLA-A | 0.349061207 |
| HLA-B | 0.347048285 |
| HLA-C | 0.346220033 |
| HLA-DMB | 0.346138368 |
| HLA-DOA | 0.346099359 |
| HLA-DPA1 | 0.345283862 |
| HLA-DQA1 | 0.344850808 |
| HLA-DQA2 | 0.343963545 |
| HLA-DRA | 0.343793274 |
| HLA-DRB1 | 0.341012381 |
| HLA-DRB5 | 0.3410012 |
| HLA-E | 0.340269137 |
| HLA-F | 0.339653035 |
| HLCS | 0.339327294 |
| HMCN2 | 0.338340285 |
| HMGB3 | 0.338126355 |
| HMGCL | 0.338035254 |
| HMGCR | 0.336684476 |
| HMGCS1 | 0.334634902 |
| HOMER1 | 0.333365411 |
| HPGDS | 0.331206161 |
| HPSE2 | 0.330593941 |
| HRASLS5 | 0.330425547 |
| HRSP12 | 0.330210898 |
| HSD11B1 | 0.330077469 |
| HSD11B2 | 0.328295015 |
| HSD17B11 | 0.328286358 |
| HSD17B13 | 0.328201223 |
| HSD17B2 | 0.327136911 |
| HSD3B1 | 0.32553856 |
| HSD3B2 | 0.324191227 |
| HSPA12A | 0.324023642 |
| HSPB6 | 0.323967547 |
| HSPB7 | 0.323844248 |
| HSPE1 | 0.323333539 |
| HSPG2 | 0.322286825 |
| HTRA3 | 0.322013216 |
| HYAL1 | 0.321623895 |
| ID2 | 0.321248741 |
| ID3 | 0.32121773 |
| IDH1 | 0.319935235 |
| IDH2 | 0.318706745 |
| IDI1 | 0.315251532 |
| IER3 | 0.314342282 |
| 1F127 | 0.310739952 |
| 1F144L | 0.308630452 |
| IFIT2 | 0.306903229 |
| IFIT3 | 0.306597217 |
| IFITM1 | 0.288684024 |
| IFITM3 | −0.300679949 |
| IFNG | −0.306736602 |
| IFRD1 | −0.309907523 |
| IGF1 | −0.31122826 |
| IGF2 | −0.311737198 |
| IGFBP3 | −0.313895641 |
| IGFL2 | −0.316021513 |
| IGFL4 | −0.316638145 |
| IGJ | −0.319191271 |
| IGLL5 | −0.319294922 |
| IGLONS | −0.321538601 |
| IGSF10 | −0.32374151 |
| IGSF6 | −0.32412571 |
| IKZF1 | −0.325523477 |
| IKZF3 | −0.327145557 |
| ILI ORA | −0.32780564 |
| IL15RA | −0.329459037 |
| IL17D | −0.32959268 |
| ILI B | −0.330397817 |
| IL1F5 | −0.331656538 |
| IL1F7 | −0.332343867 |
| IL2RG | −0.333512614 |
| IL32 | −0.337044685 |
| IL33 | −0.337543924 |
| IL7R | −0.3402628 |
| IL8 | −0.340327434 |
| ILDR1 | −0.344833085 |
| INHBB | −0.345793364 |
| INMT | −0.346780353 |
| INPPL1 | −0.347810097 |
| INSIG1 | −0.348758707 |
| INSM1 | −0.349648393 |
| IQCJ | −0.350708871 |
| IQSEC3 | −0.351774633 |
| IRF1 | −0.352437557 |
| IRF8 | −0.354239737 |
| IRGM | −0.354786194 |
| IRX6 | −0.354863011 |
| ISLR | −0.356012151 |
| ISM1 | −0.356364283 |
| ISOC1 | −0.356612572 |
| ITGA1 | −0.357737902 |
| ITGA2 | −0.358686744 |
| ITGA7 | −0.35885742 |
| ITGA8 | −0.359303654 |
| ITGAL | −0.359561236 |
| ITGAM | −0.360274694 |
| ITGB1BP1 | −0.361124559 |
| ITGB2 | −0.362379255 |
| ITIH5 | −0.362861158 |
| ITK | −0.364220441 |
| ITSN1 | −0.364603478 |
| IVD | −0.364874994 |
| IYD | −0.367247619 |
| JAM3 | −0.369137152 |
| JPH2 | −0.36915775 |
| KANK2 | −0.36939919 |
| KCNA3 | −0.369814629 |
| KCNA6 | −0.370700895 |
| KCNB1 | −0.372800404 |
| KCNH5 | −0.373558632 |
| KCNIP2 | −0.374188703 |
| KCNIP3 | −0.37554618 |
| KCNJ13 | −0.375904647 |
| KCNJ15 | −0.376715272 |
| KCNK2 | −0.377005302 |
| KCNMA1 | −0.377366219 |
| KCNMB1 | −0.378737683 |
| KDELC2 | −0.380063597 |
| KIAA0513 | −0.380251395 |
| KIAA0754 | −0.38043491 |
| KIAA1191 | −0.380825884 |
| KIF1A | −0.381197718 |
| KIF3C | −0.383830723 |
| KIRREL | −0.383840228 |
| KLHDC8B | −0.384186674 |
| KLHL31 | −0.384530502 |
| KLK10 | −0.386465555 |
| KLK12 | −0.386788535 |
| KLK13 | −0.38708881 |
| KLK6 | −0.389841088 |
| KLRF1 | −0.391717697 |
| KPRP | −0.392960148 |
| KREMEN2 | −0.393129417 |
| KRT16 | −0.394071465 |
| KRT17 | −0.395471411 |
| KRT20 | −0.396530359 |
| KRT25 | −0.397452929 |
| KRT26 | −0.399757999 |
| KRT27 | −0.400126125 |
| KRT28 | −0.401030923 |
| KRT31 | −0.402427006 |
| KRT32 | −0.403200561 |
| KRT33A | −0.403652835 |
| KRT33B | −0.404109387 |
| KRT34 | −0.405025997 |
| KRT35 | −0.406442519 |
| KRT36 | −0.407149843 |
| KRT38 | −0.40718969 |
| KRT39 | −0.409800723 |
| KRT4 | −0.410615619 |
| KRT40 | −0.411781704 |
| KRT6A | −0.413538794 |
| KRT6B | −0.415093538 |
| KRT6C | −0.415339343 |
| KRT71 | −0.415595423 |
| KRT72 | −0.415633737 |
| KRT73 | −0.417054693 |
| KRT74 | −0.417810039 |
| KRT75 | −0.418723877 |
| KRT79 | −0.418809139 |
| KRT81 | −0.418869716 |
| KRT82 | −0.41909923 |
| KRT83 | −0.420227024 |
| KRT84 | −0.420443314 |
| KRT85 | −0.420571265 |
| KRT86 | −0.421084266 |
| KRT9 | −0.421204889 |
| KRTAP1-1 | −0.422153235 |
| KRTAP1-3 | −0.422315859 |
| KRTAP1-5 | −0.422353098 |
| KRTAP10-1 | −0.423023706 |
| KRTAP10-10 | −0.424099277 |
| KRTAP10-11 | −0.424301257 |
| KRTAP10-12 | −0.424712177 |
| KRTAP10-2 | −0.427539756 |
| KRTAP10-3 | −0.427689941 |
| KRTAP10-4 | −0.427750966 |
| KRTAP10-5 | −0.430626916 |
| KRTAP10-6 | −0.430914189 |
| KRTAP10-7 | −0.433469035 |
| KRTAP10-8 | −0.434354078 |
| KRTAP10-9 | −0.434929572 |
| KRTAP11-1 | −0.435501701 |
| KRTAP12-1 | −0.435712912 |
| KRTAP12-2 | −0.43660115 |
| KRTAP12-3 | −0.439409414 |
| KRTAP13-1 | −0.440550728 |
| KRTAP13-2 | −0.440552465 |
| KRTAP17-1 | −0.442502314 |
| KRTAP19-1 | −0.443567733 |
| KRTAP19-3 | −0.444118895 |
| KRTAP19-5 | −0.444688563 |
| KRTAP19-7 | −0.445067557 |
| KRTAP2-1 | −0.44562324 |
| KRTAP2-2 | −0.445882534 |
| KRTAP2-4 | −0.446217959 |
| KRTAP20-1 | −0.447316938 |
| KRTAP20-2 | −0.448114149 |
| KRTAP21-2 | −0.449069292 |
| KRTAP24-1 | −0.449626039 |
| KRTAP26-1 | −0.451163592 |
| KRTAP3-1 | −0.45159612 |
| KRTAP3-2 | −0.451669157 |
| KRTAP3-3 | −0.452247925 |
| KRTAP4-1 | −0.453979991 |
| KRTAP4-11 | −0.454790804 |
| KRTAP4-12 | −0.456253068 |
| KRTAP4-2 | −0.458620363 |
| KRTAP4-3 | −0.459015958 |
| KRTAP4-4 | −0.459349895 |
| KRTAP4-5 | −0.462866769 |
| KRTAP4-6 | −0.464251992 |
| KRTAP4-7 | −0.466986043 |
| KRTAP4-8 | −0.468424448 |
| KRTAP4-9 | −0.468658451 |
| KRTAP5-1 | −0.471737002 |
| KRTAP5-10 | −0.471879909 |
| KRTAP5-11 | −0.473314254 |
| KRTAP5-2 | −0.474031573 |
| KRTAP5-3 | −0.474907372 |
| KRTAP5-4 | −0.475958446 |
| KRTAP5-5 | −0.47632523 |
| KRTAP5-6 | −0.477968056 |
| KRTAP5-7 | −0.47862366 |
| KRTAP5-8 | −0.478890592 |
| KRTAP5-9 | −0.47901515 |
| KRTAP7-1 | −0.479719477 |
| KRTAP8-1 | −0.481831618 |
| KRTAP9-2 | −0.484057735 |
| KRTAP9-3 | −0.484188477 |
| KRTAP9-4 | −0.484820257 |
| KRTAP9-8 | −0.485086719 |
| KRTAP9-9 | −0.486003575 |
| LAIR1 | −0.486475554 |
| LAMA2 | −0.486595848 |
| LAMA4 | −0.48707544 |
| LAMB4 | −0.487651046 |
| LAMC1 | −0.488390249 |
| LAPTM5 | −0.488636639 |
| LASS4 | −0.48991999 |
| LAT | −0.490092018 |
| LAX1 | −0.490639187 |
| LBP | −0.493496313 |
| LCE1A | −0.494128776 |
| LCE2A | −0.495219438 |
| LCE2B | −0.49527651 |
| LCE2C | −0.496493358 |
| LCE5A | −0.496747849 |
| LCK | −0.496927272 |
| LCN2 | −0.497751507 |
| LCP1 | −0.497755673 |
| LCP2 | −0.50379876 |
| LCT | −0.504815734 |
| LDHB | −0.50717027 |
| LDHD | −0.50742416 |
| LDLR | −0.509358508 |
| LEP | −0.509546106 |
| LEPR | −0.512238623 |
| LETM1 | −0.512551133 |
| LGALS9 | −0.513575251 |
| LGI3 | −0.514039127 |
| LGR5 | −0.514482089 |
| LHFP | −0.515549919 |
| LHFPL4 | −0.515645851 |
| LHX2 | −0.517205628 |
| LHX3 | −0.51886187 |
| LILRB5 | −0.519507453 |
| LIMCH1 | −0.520351478 |
| LIPE | −0.523394371 |
| LIPG | −0.523716319 |
| LIPH | −0.524508068 |
| LIPK | −0.524701057 |
| LMO3 | −0.525923847 |
| LMOD1 | −0.527767234 |
| LOX | −0.529750106 |
| LPCAT3 | −0.530850216 |
| LPHN3 | −0.532483446 |
| LPIN1 | −0.535930759 |
| LPL | −0.535974399 |
| LPO | −0.539960832 |
| LRG1 | −0.540227863 |
| LRIT2 | −0.540659866 |
| LRP1 | −0.541122408 |
| LRRC1OB | −0.541382981 |
| LRRC31 | −0.541908609 |
| LRRC32 | −0.542449599 |
| LRRN4CL | −0.544401271 |
| LTB | −0.545811456 |
| LTBP2 | −0.546255227 |
| LTBP3 | −0.546520759 |
| LTF | −0.546576967 |
| LUM | −0.547037791 |
| LY6E | −0.547871644 |
| LY6G6D | −0.548587941 |
| LY9 | −0.548592091 |
| LYG2 | −0.550622286 |
| LYRM5 | −0.553750184 |
| LYZ | −0.553908798 |
| LZTS1 | −0.55470368 |
| MAF | −0.555326479 |
| MAGEAll | −0.560324601 |
| MAGEB2 | −0.561237931 |
| MAN1A1 | −0.564424533 |
| MAN2B2 | −0.564616419 |
| MANEAL | −0.568102524 |
| MAOB | −0.569761253 |
| MAP1B | −0.57111945 |
| MAPK8IP2 | −0.573871012 |
| MARCKSL1 | −0.574963966 |
| MARCO | −0.575413522 |
| MAT1A | −0.576230552 |
| MBOAT2 | −0.578483541 |
| MC5R | −0.578631006 |
| MCAM | −0.578789415 |
| MCCC1 | −0.581881171 |
| MCCC2 | −0.581966797 |
| ME1 | −0.583729688 |
| MECR | −0.584619372 |
| MEF2A | −0.584700412 |
| MEGF11 | −0.584921065 |
| MERTK | −0.586178861 |
| METTL7B | −0.587425188 |
| MFAP3L | −0.588407811 |
| MFAP4 | −0.588595975 |
| MFAP5 | −0.58896597 |
| MFGE8 | −0.589276627 |
| MGLL | −0.590090765 |
| MGP | −0.593019469 |
| MGST1 | −0.5931126 |
| MKX | −0.594489105 |
| MLANA | −0.598565645 |
| MMD | −0.600014427 |
| MME | −0.600332202 |
| MMP28 | −0.600345509 |
| MMP7 | −0.600647859 |
| MMP9 | −0.602399345 |
| MMRN1 | −0.603841554 |
| MNDA | −0.603900561 |
| MOCOS | −0.604251074 |
| MOGAT1 | −0.604699311 |
| MOGAT2 | −0.605613414 |
| MORN2 | −0.605797707 |
| MPEG1 | −0.606068102 |
| MRC1 | −0.606250609 |
| MRC1L1 | −0.60934354 |
| MRPL13 | −0.611878842 |
| MRPL14 | −0.613933735 |
| MRPS25 | −0.613995035 |
| MRVI1 | −0.614302775 |
| MS4A1 | −0.615988158 |
| MS4A18 | −0.617064793 |
| MS4A4A | −0.623456154 |
| MS4A6A | −0.623584643 |
| MS4A7 | −0.624896318 |
| MSMB | −0.626182891 |
| MSR1 | −0.626658615 |
| MTCH2 | −0.62705467 |
| MTOR | −0.628236911 |
| MUC1 | −0.629292281 |
| MUC15 | −0.629668792 |
| MUC21 | −0.630097959 |
| MUC7 | −0.630707817 |
| MUCL1 | −0.632281702 |
| MUM1L1 | −0.632890347 |
| MVD | −0.63377999 |
| MVK | −0.634350771 |
| MX1 | −0.637154959 |
| MX2 | −0.640063673 |
| MXRA7 | −0.641897514 |
| MXRA8 | −0.642027129 |
| MYBPC1 | −0.642289244 |
| MYEOV | −0.643709762 |
| MYH11 | −0.6445285 |
| MYH3 | −0.645671444 |
| MYL9 | −0.64852348 |
| MY01F | −0.653656081 |
| MY01G | −0.655947427 |
| MYOCD | −0.656003739 |
| MYOM2 | −0.656204738 |
| MYOZ2 | −0.657402926 |
| NAALAD2 | −0.658531502 |
| NADSYN1 | −0.65914615 |
| NAMPT | −0.663321221 |
| NCKAP1L | −0.66395007 |
| NDRG1 | −0.665366494 |
| NDUFA4 | −0.667554244 |
| NDUFA4L2 | −0.667734019 |
| NDUFC2 | −0.66933108 |
| NDUFSS | −0.670809021 |
| NEFH | −0.671330604 |
| NEFL | −0.67286048 |
| NEGR1 | −0.673876365 |
| NET01 | −0.681627045 |
| NETO2 | −0.681765683 |
| NEXN | −0.689608187 |
| NFKBIZ | −0.690630636 |
| NID1 | −0.691279966 |
| NIT2 | −0.691320912 |
| NKG7 | −0.691603556 |
| NLRP10 | −0.694640544 |
| NOP16 | −0.696609698 |
| NOTCH1 | −0.700702492 |
| NOVA1 | −0.703190588 |
| NOX4 | −0.705313267 |
| NPC1 | −0.707841851 |
| NPL | −0.709858759 |
| NPR1 | −0.710007842 |
| NPR2 | −0.711393968 |
| NQO2 | −0.714002827 |
| NR1H3 | −0.716413515 |
| NRN1 | −0.718708902 |
| NRP2 | −0.719576908 |
| NSDHL | −0.72289418 |
| MYL4 | −0.64848487 |
| NTM | −0.724714231 |
| NTN1 | −0.726146775 |
| NTRK2 | −0.72724064 |
| NUDT4 | −0.730602904 |
| NUS1 | −0.732682929 |
| NXF3 | −0.737566122 |
| NXPH4 | −0.741144139 |
| OAS1 | −0.74252223 |
| OAS2 | −0.744908106 |
| OASL | −0.748125007 |
| 0C90 | −0.748786894 |
| OCA2 | −0.749872404 |
| ODZ3 | −0.753954128 |
| OLAH | −0.754127692 |
| OLFM4 | −0.756232771 |
| OLFML3 | −0.758876338 |
| OLIG3 | −0.761588941 |
| OR52E8 | −0.761733745 |
| OR7E24 | −0.763566491 |
| OSGIN2 | −0.768662816 |
| OTUB2 | −0.769525642 |
| OXCT2 | −0.770343029 |
| P2RY12 | −0.772886519 |
| P2RY4 | −0.774171979 |
| PADI3 | −0.775142684 |
| PADI4 | −0.776070731 |
| PAFAH2 | −0.776122957 |
| PALM2-AKAP2 | −0.777965777 |
| PAM | −0.778145916 |
| PAMR1 | −0.778764169 |
| PANK1 | −0.783102787 |
| PAPLN | −0.789506373 |
| PAQR3 | −0.789741862 |
| PARP14 | −0.789834158 |
| PARVB | −0.792557647 |
| PC | −0.792618446 |
| PCCB | −0.793676467 |
| PCDH12 | −0.794838328 |
| PCDH18 | −0.795317977 |
| PCDH9 | −0.797143303 |
| NTAN1 | −0.724030746 |
| PCK1 | −0.798762701 |
| PCK2 | −0.80093061 |
| PCOLCE2 | −0.805749221 |
| PCP4 | −0.80660577 |
| PCP4L1 | −0.810182604 |
| PCSK2 | −0.812833431 |
| PCSK6 | −0.814519517 |
| PCTP | −0.815880263 |
| PCYT2 | −0.815967921 |
| PDCD1 | −0.817628588 |
| PDCD1LG2 | −0.819107555 |
| PDE2A | −0.819209688 |
| PDE3B | −0.819462683 |
| PDE4DIP | −0.823261269 |
| PDE6A | −0.823388941 |
| PDGFC | −0.824566198 |
| PDGFD | −0.825600768 |
| PDGFRA | −0.82681899 |
| PDGFRB | −0.827548745 |
| PDHA1 | −0.827581041 |
| PDHX | −0.830580639 |
| PDLIM3 | −0.831618564 |
| PDZD4 | −0.832261007 |
| PDZK1 | −0.833857225 |
| PDZRN4 | −0.835885008 |
| PEB P4 | −0.836770014 |
| PECR | −0.841763001 |
| PENK | −0.843364546 |
| PEX11A | −0.844148412 |
| PEX16 | −0.845240619 |
| PEX3 | −0.845557198 |
| PFKFB 1 | −0.84580667 |
| PFKFB4 | −0.845949888 |
| PFN2 | −0.85097388 |
| PGBD5 | −0.852325721 |
| PGD | −0.854690728 |
| PGM1 | −0.855162254 |
| PGM5 | −0.857334174 |
| PGRMC1 | −0.858260065 |
| PHLDA2 | −0.861203361 |
| PHLDB1 | −0.861641022 |
| PHLDB2 | −0.862718347 |
| PHYHIP | −0.86487347 |
| PI15 | −0.865029734 |
| PI16 | −0.865665115 |
| PID1 | −0.866498848 |
| PIP | −0.866519334 |
| PIRT | −0.867397765 |
| PKDCC | −0.867670074 |
| PKIB | −0.869154885 |
| PKLR | −0.869881586 |
| PLA2G2A | −0.876605146 |
| PLA2G2F | −0.879575732 |
| PLA2G7 | −0.881039596 |
| PLA2R1 | −0.883520743 |
| PLAU | −0.886112091 |
| PLCL2 | −0.887857416 |
| PLD5 | −0.88833026 |
| PLEK | −0.890588014 |
| PLEKHH1 | −0.892013042 |
| PLEKHO2 | −0.892759062 |
| PLIN1 | −0.897514794 |
| PLIN2 | −0.898322981 |
| PLIN4 | −0.900751318 |
| PLIN5 | −0.908571912 |
| PLN | −0.913817925 |
| PLP1 | −0.91474431 |
| PLXDC1 | −0.915580511 |
| PLXNA4 | −0.917886802 |
| PLXND1 | −0.91906664 |
| PM20D1 | −0.921459907 |
| PMEPAI | −0.931379152 |
| PMFBP1 | −0.932648545 |
| PMM1 | −0.935248795 |
| PMVK | −0.935929055 |
| PNLDC1 | −0.937171756 |
| PNLIPRP2 | −0.937889074 |
| PNLIPRP3 | −0.940702839 |
| PNMAL1 | −0.946545491 |
| PNPLA3 | −0.949471247 |
| PNPLA5 | −0.949827267 |
| PODN | −0.950509276 |
| PODXL2 | −0.950993074 |
| PON3 | −0.953720157 |
| POPDC2 | −0.958332824 |
| POR | −0.962594839 |
| POSTN | −0.962601556 |
| POU3F1 | −0.963636244 |
| PPAP2B | −0.964939925 |
| PPARG | −0.965940879 |
| PPARGC1A | −0.966194123 |
| PPBP | −0.966890036 |
| PPIF | −0.969772394 |
| PPM1K | −0.970342274 |
| PPP1R12B | −0.97072505 |
| PPP1R1A | −0.971418181 |
| PPP1R3G | −0.972776135 |
| PPP2R2B | −0.976401971 |
| PPP2R2C | −0.977204221 |
| PPP2R4 | −0.977337731 |
| PPP4R4 | −0.979654264 |
| PPT2 | −0.981991149 |
| PRAME | −0.982312375 |
| PRB1 | −0.994692811 |
| PRB2 | −0.994837623 |
| PRB3 | −0.996215922 |
| PRDX2 | −0.999361653 |
| PRELP | −1.003532216 |
| PRF1 | −1.005068627 |
| PRG4 | −1.005650622 |
| PRKAR2B | −1.008960821 |
| PRKCB | −1.009564242 |
| PRKCQ | −1.022108105 |
| PRR4 | −1.022122141 |
| PRR5L | −1.027649291 |
| PRRT4 | −1.031008711 |
| PRS S22 | −1.032236383 |
| PRS S27 | −1.033034894 |
| PRSS8 | −1.033578955 |
| PRUNE2 | −1.033678655 |
| PSAPL1 | −1.036430499 |
| PSG4 | −1.042194601 |
| PSMA4 | −1.042727531 |
| PSMB9 | −1.044534141 |
| PSME4 | −1.046889284 |
| PSPH | −1.048171908 |
| PTGDS | −1.054223374 |
| PTGER3 | −1.054992441 |
| PTGFR | −1.056299121 |
| PTGIS | −1.05754987 |
| PTHLH | −1.059510627 |
| PTN | −1.06250376 |
| PTPN7 | −1.063104801 |
| PTPRC | −1.063584427 |
| PTPRCAP | −1.071850449 |
| PTPRG | −1.073370158 |
| PTX3 | −1.074808622 |
| PXDN | −1.074890432 |
| PXMP2 | −1.075601876 |
| PXMP4 | −1.077986692 |
| PXN | −1.08034317 |
| PYHIN1 | −1.083848362 |
| RAB 15 | −1.084824209 |
| RAC2 | −1.086828971 |
| RAC3 | −1.090243631 |
| RAG1AP1 | −1.091339717 |
| RAMP2 | −1.096355468 |
| RARRES 1 | −1.098789796 |
| RARRES 2 | −1.100435776 |
| RASA3 | −1.102829903 |
| RASAL1 | −1.105642611 |
| RASEF | −1.106833599 |
| RASSF3 | −1.107042294 |
| RBM47 | −1.111771937 |
| RBMS3 | −1.116827197 |
| RBP1 | −1.117753239 |
| RBP4 | −1.118375901 |
| RBP5 | −1.12276955 |
| RCL1 | −1.124403406 |
| RDH11 | −1.126509335 |
| RDH5 | −1.127828066 |
| RECK | −1.128681054 |
| REEP6 | −1.135067016 |
| REG3G | −1.13607941 |
| RETS AT | −1.136196498 |
| RFTN1 | −1.141486756 |
| RGL1 | −1.144105792 |
| RGS9BP | −1.145528397 |
| RHCG | −1.15017106 |
| RHOXF2 | −1.151218004 |
| RIMBP2 | −1.15144067 |
| RIMS4 | −1.156038088 |
| RNASE1 | −1.161880853 |
| RND3 | −1.168535655 |
| RNF133 | −1.171186627 |
| RNF157 | −1.173390837 |
| RNF17 | −1.174078767 |
| RNFT1 | −1.176140661 |
| ROBO3 | −1.180703761 |
| ROBO4 | −1.185319691 |
| ROS1 | −1.185769523 |
| RRAS | −1.187642266 |
| RSAD2 | −1.202381334 |
| RSPO3 | −1.202461536 |
| RTN1 | −1.202839214 |
| S100A2 | −1.204881512 |
| S100A3 | −1.210729144 |
| S100A7 | −1.211199562 |
| S100A7A | −1.217930597 |
| S100A8 | −1.219924354 |
| S100A9 | −1.22153891 |
| SlOOP | −1.228258867 |
| S1PR4 | −1.239866405 |
| SAA1 | −1.246475942 |
| SAMD3 | −1.248544948 |
| S AMD5 | −1.252026697 |
| SAMD9L | −1.252631907 |
| SAMHD1 | −1.258847859 |
| SAMM50 | −1.264424403 |
| SAMSN1 | −1.27101594 |
| SASH3 | −1.273634382 |
| SATB2 | −1.275282037 |
| SC4MOL | −1.28096868 |
| SC5DL | −1.286463995 |
| SCAP | −1.287456105 |
| SCARA5 | −1.291877483 |
| SCGB1D2 | −1.293284043 |
| SCGB2A1 | −1.294232926 |
| SCGB2A2 | −1.294899427 |
| SCGB3A1 | −1.296468343 |
| SCGBL | −1.301208943 |
| SCN2A | −1.301250876 |
| SCN7A | −1.302020896 |
| SCUBE2 | −1.309935328 |
| SDPR | −1.31280231 |
| SEC11C | −1.320019492 |
| SEC14L2 | −1.322084576 |
| SEC14L4 | −1.324121885 |
| SELPLG | −1.326107878 |
| SEMA3A | −1.327980096 |
| SEMA5B | −1.328788292 |
| SEMA6B | −1.329815059 |
| SEMA7A | −1.329854619 |
| SEPX1 | −1.330747335 |
| SERINC2 | −1.331710304 |
| SERPINA12 | −1.332317249 |
| SERPINA3 | −1.334147653 |
| SERPINA4 | −1.340571535 |
| SERPINA9 | −1.340758174 |
| SERPINB13 | −1.351490104 |
| SERPINB3 | −1.352269517 |
| SERPINB7 | −1.369123951 |
| SERPINE2 | −1.37556256 |
| SERPINF1 | −1.378089143 |
| SERPING1 | −1.378730149 |
| SETBP1 | −1.383530305 |
| SFRP5 | −1.383881515 |
| SGCD | −1.389073384 |
| SGCG | −1.389145348 |
| SGK2 | −1.396956168 |
| SGPP2 | −1.399191576 |
| SH2B3 | −1.400617189 |
| SH2D1A | −1.400978601 |
| SH3KBP1 | −1.402700511 |
| SHF | −1.40345822 |
| SHISA2 | −1.403579535 |
| SHISA6 | −1.404419786 |
| SIGLECI | −1.413143194 |
| SILV | −1.415567601 |
| SIPA1L2 | −1.416946804 |
| SIT1 | −1.420571252 |
| SLA | −1.422758907 |
| SLAMF1 | −1.434051444 |
| SLAMF6 | −1.439359495 |
| SLAMF8 | −1.441764556 |
| SLC11A2 | −1.449195961 |
| SLC12A8 | −1.453723191 |
| SLC13A4 | −1.455480788 |
| SLC15A1 | −1.456321902 |
| SLC15A2 | −1.456330876 |
| SLC16A11 | −1.471379407 |
| SLC16A6 | −1.472478376 |
| SLC16A9 | −1.477438555 |
| SLC24A5 | −1.478304999 |
| SLC25A1 | −1.483078735 |
| SLC25A11 | −1.490907721 |
| SLC25A17 | −1.491233549 |
| SLC25A18 | −1.500692617 |
| SLC25A33 | −1.508439265 |
| SLC25A34 | −1.510180835 |
| SLC25A35 | −1.521499497 |
| SLC25A41 | −1.528579196 |
| SLC25A5 | −1.530879501 |
| SLC26A2 | −1.542114303 |
| SLC26A3 | −1.547375232 |
| SLC26A6 | −1.548584857 |
| SLC26A9 | −1.549961186 |
| SLC27A2 | −1.557902316 |
| SLC27A4 | −1.559401915 |
| SLC27A6 | −1.5654479 |
| SLC2A1 | −1.573091222 |
| SLC2A14 | −1.589765601 |
| SLC2A9 | −1.59010157 |
| SLC30A1 | −1.608266432 |
| SLC30A10 | −1.610688839 |
| SLC31A1 | −1.618876818 |
| SLC31A2 | −1.627863869 |
| SLC35A2 | −1.646355483 |
| SLC35B1 | −1.658205688 |
| SLC39A8 | −1.664362866 |
| SLC3A1 | −1.666990786 |
| SLC40A1 | −1.66745607 |
| SLC41A1 | −1.671905963 |
| SLC43A2 | −1.673561679 |
| SLC43A3 | −1.675409775 |
| SLC44A3 | −1.681463091 |
| SLC45A4 | −1.688290291 |
| SLC46A3 | −1.693262945 |
| SLC5A6 | −1.694868645 |
| SLC6A14 | −1.694980582 |
| SLC6A17 | −1.696211048 |
| SLC7A4 | −1.700004122 |
| SLC7A5 | −1.704302363 |
| SLC9A2 | −1.712239497 |
| SLCO4C1 | −1.721723356 |
| SLFN5 | −1.723054992 |
| SLIT2 | −1.727876643 |
| SLIT3 | −1.733374301 |
| SMAD3 | −1.734235574 |
| SMAD6 | −1.748938393 |
| SMOC1 | −1.754124909 |
| SMOC2 | −1.754436958 |
| SMOX | −1.756787067 |
| SMTN | −1.761983438 |
| SNCG | −1.764621533 |
| SNED1 | −1.772625098 |
| SNX10 | −1.774826682 |
| SNX4 | −1.776375659 |
| SOAT1 | −1.792226627 |
| SOD3 | −1.792817995 |
| SORBS1 | −1.832013019 |
| SORD | −1.835918336 |
| SOST | −1.841448702 |
| SOSTDC1 | −1.841936647 |
| SOX7 | −1.863023833 |
| SOX9 | −1.870326656 |
| SP5 | −1.878132382 |
| SP8 | −1.895852716 |
| SPARC | −1.922410453 |
| SPEG | −1.929695412 |
| SPI1 | −1.930789372 |
| SPIRE2 | −1.932670971 |
| SPNS2 | −1.940225497 |
| SPOCK1 | −1.952454013 |
| SPOCK2 | −1.96017109 |
| SPP1 | −1.977911033 |
| SPRR1A | −1.979883678 |
| SPRR1B | −1.980951653 |
| SPRR2A | −1.984424997 |
| SPRR2B | −1.986219304 |
| SPRR2D | −2.000266228 |
| SPRR4 | −2.001006227 |
| SPRY1 | −2.003800549 |
| SPTBN5 | −2.023808705 |
| SRD5A1 | −2.042190884 |
| SRD5A3 | −2.0486371 |
| SREBF1 | −2.049456335 |
| SRGN | −2.051287523 |
| SRMS | −2.058739041 |
| SRPRB | −2.070288076 |
| SRPX | −2.071574089 |
| SRXN1 | −2.081292197 |
| SSPN | −2.088982204 |
| ST6GALNAC4 | −2.093052078 |
| STAB2 | −2.09531773 |
| STAP2 | −2.09877818 |
| STARD4 | −2.10081125 |
| STAT1 | −2.107321065 |
| STAT5A | −2.111738883 |
| STEAP4 | −2.140314132 |
| STK17B | −2.146015883 |
| STRBP | −2.149553645 |
| STS | −2.169730852 |
| STXBP5L | −2.178055522 |
| SULT1C3 | −2.182767281 |
| SULT4A1 | −2.185137619 |
| SUOX | −2.191531684 |
| SUSD4 | −2.196094944 |
| SVEP1 | −2.19690447 |
| SYN2 | −2.19910339 |
| SYNE1 | −2.203113551 |
| SYNM | −2.206267688 |
| SYNPO | −2.209830988 |
| SYNPO2 | −2.217753514 |
| SYPL2 | −2.233018178 |
| SYT12 | −2.237249742 |
| SYT4 | −2.240487205 |
| SYT7 | −2.246464548 |
| SYTL5 | −2.247408935 |
| TAC1 | −2.248448175 |
| TACC1 | −2.262090835 |
| TACR2 | −2.269897864 |
| TAGLN | −2.282062492 |
| TAGLN3 | −2.287657256 |
| TAP1 | −2.289726386 |
| TAS1R3 | −2.294200626 |
| TBX5 | −2.296211382 |
| TCHH | −2.297991423 |
| TCHHLI | −2.300359116 |
| TDRD12 | −2.302104844 |
| TDRD6 | −2.303133347 |
| TECR | −2.304168293 |
| TECRL | −2.32737199 |
| TENC1 | −2.328788419 |
| TEX264 | −2.32931955 |
| TF | −2.330864878 |
| TFF3 | −2.338234361 |
| TFPI | −2.345658305 |
| TFRC | −2.352048995 |
| TG | −2.388666049 |
| TGFB1 | −2.39481561 |
| TGFBR3 | −2.432778299 |
| TGM6 | −2.447909311 |
| TGM7 | −2.455059154 |
| THBS2 | −2.462338256 |
| THEMIS | −2.466821465 |
| THRSP | −2.46803843 |
| TIGIT | −2.469110549 |
| TIMM8B | −2.471357542 |
| TIMP3 | −2.472940088 |
| TIMP4 | −2.48477281 |
| TKT | −2.502497665 |
| TLCD1 | −2.507532929 |
| TLCD2 | −2.518026735 |
| TLE2 | −2.521998446 |
| TLN1 | −2.538296154 |
| TLN2 | −2.550059361 |
| TM4SF1 | −2.554078171 |
| TM7SF2 | −2.558357723 |
| TMC2 | −2.577931898 |
| TMC5 | −2.597892642 |
| TMC8 | −2.601271973 |
| TMCC3 | −2.612083339 |
| TMED3 | −2.613928335 |
| TMED5 | −2.62446575 |
| TMEM132C | −2.631317468 |
| TMEM135 | −2.658803942 |
| TMEM164 | −2.66469741 |
| TMEM171 | −2.673694271 |
| TMEM176A | −2.697627918 |
| TMEM176B | −2.705925879 |
| TMEM189 | −2.709598019 |
| TMEM33 | −2.711755193 |
| TMEM37 | −2.743251825 |
| TMEM41A | −2.749305206 |
| TMEM56 | −2.758633885 |
| TMEM63C | −2.778244179 |
| TMEM91 | −2.794404326 |
| TMEM97 | −2.801713989 |
| TMPRSS11E | −2.80608481 |
| TNFAIP2 | −2.826247307 |
| TNFRSF1B | −2.860023569 |
| TNMD | −2.8645123 |
| TNNT1 | −2.871825177 |
| TNNT2 | −2.896324562 |
| TNR | −2.941774511 |
| TNS1 | −2.945317659 |
| TNS3 | −2.971814537 |
| TNXB | −2.994004996 |
| TP53INP2 | −3.009811411 |
| TPD52L1 | −3.04022385 |
| TPM2 | −3.062897334 |
| TPSAB1 | −3.066487578 |
| TPSB2 | −3.07097506 |
| TRANK1 | −3.072990775 |
| TRAT1 | −3.094221481 |
| TRIL | −3.112996059 |
| TRIM17 | −3.123494767 |
| TRIM2 | −3.124628905 |
| TRIM22 | −3.133403557 |
| TRIM48 | −3.138698017 |
| TRIM55 | −3.165011459 |
| TRIM58 | −3.181998773 |
| TRIM67 | −3.207252697 |
| TRIML2 | −3.271088667 |
| TRIO | −3.29291105 |
| TRPS1 | −3.316145919 |
| TSHZ2 | −3.31819321 |
| TSPAN2 | −3.349420414 |
| TSPAN6 | −3.380391852 |
| TST | −3.393068288 |
| TTC38 | −3.405295975 |
| TTC39B | −3.40938693 |
| TTLL4 | −3.451027458 |
| TTYH3 | −3.453294497 |
| TUSC5 | −3.455257937 |
| TXN | −3.479541946 |
| TXNDC9 | −3.480231662 |
| TYR | −3.480735135 |
| TYRP1 | −3.500146676 |
| UBA7 | −3.507548799 |
| UBASH3A | −3.521909062 |
| UBD | −3.533653454 |
| UBE2L6 | −3.535852084 |
| UBE2QL1 | −3.558825001 |
| UBE2S | −3.560413169 |
| UBIAD1 | −3.568701272 |
| UBR4 | −3.577711909 |
| UGT1A4 | −3.610729727 |
| UGT1A6 | −3.615822399 |
| UGT2A1 | −3.62388291 |
| UGT2A2 | −3.624037214 |
| UGT3A2 | −3.684967762 |
| ULK4 | −3.69391787 |
| UNCSB | −3.706039835 |
| UPB1 | −3.733419079 |
| UQCRC1 | −3.756595137 |
| UQCRH | −3.759996943 |
| UQCRQ | −3.761985111 |
| USMG5 | −3.774235021 |
| VCAM1 | −3.77691507 |
| VGLL3 | −3.788116794 |
| VIP | −3.79667408 |
| VIT | −3.811544878 |
| VLDLR | −3.815181583 |
| VNN1 | −3.834888027 |
| VNN2 | −3.859797541 |
| VSIG4 | −3.881799111 |
| VSNL1 | −3.883629969 |
| VTCN1 | −3.900191403 |
| VWF | −3.917152093 |
| WASF3 | −3.92041248 |
| WDFY4 | −3.923083042 |
| WDR72 | −3.923108486 |
| WFDC5 | −3.935058036 |
| WFDC6 | −3.961114641 |
| WIPF1 | −3.992939617 |
| WNT3 | −4.014303311 |
| WNT4 | −4.020110524 |
| WNT7B | −4.022985537 |
| WNT9B | −4.022999702 |
| WSCD2 | −4.025740758 |
| XAGE5 | −4.025945984 |
| XBP1 | −4.048938908 |
| XPNPEP2 | −4.107938592 |
| XYLB | −4.177052766 |
| ZBED2 | −4.205448209 |
| ZC3H12A | −4.239150084 |
| ZCCHC24 | −4.392112936 |
| ZEB1 | −4.427362372 |
| ZFHX3 | −4.485302641 |
| ZFHX4 | −4.492516599 |
| ZFPM2 | −4.502949665 |
| ZG16B | −4.554145212 |
| ZIC1 | −4.592714118 |
| ZIC4 | −4.833821498 |
| ZNF117 | −4.856367277 |
| ZNF254 | −4.971443263 |
| ZNF385B | −5.057326146 |
| ZNF43 | −5.186763686 |
| ZNF493 | −5.587177539 |
| ZNF703 | −5.657126133 |
| ZNF80 | −5.722284476 |
| ZNF831 | −6.216622384 |
| ZP1 | −6.537478789 |

TABLE 5
List of TOP 35 gene signatures/regulatory factors controlling the expression of
skin-aging-related genes in various genetic networks

| Symbol | Name | HGNC ID |
|---|---|---|
| IL6 | interleukin 6 (interferon, beta 2) | HGNC:6018 |
| CSF3 | colony stimulating factor 3 (granulocyte) | HGNC:2438 |
| IFNG | interferon, gamma | HGNC:5438 |
| IL8 | interleukin 8 | HGNC:6025 |
| CXCL2 | chemokine (C-X-C motif) ligand 2 | HGNC:4603 |
| CXCL9 | chemokine (C-X-C motif) ligand 9 | HGNC:7098 |
| CXCL10 | chemokine (C-X-C motif) ligand 10 | HGNC:10637 |
| CXCL1 | chemokine (C-X-C motif) ligand 1 (melanoma growth | HGNC:4602 |
| | stimulating activity, alpha) | |
| NR4A1 | nuclear receptor subfamily 4, group A, member 1 | HGNC:7980 |
| SOCS3 | suppressor of cytokine signaling 3 | HGNC:19391 |
| SAA4 | serum amyloid A4, constitutive | HGNC:10516 |
| PCK1 | phosphoenolpyruvate carboxykinase 1 (soluble) | HGNC:8724 |
| LIPE | lipase, hormone-sensitive | HGNC:6621 |
| LEP | leptin | HGNC:6553 |
| CXCL11 | chemokine (C-X-C motif) ligand 11 | HGNC:10638 |
| CCL4L2 | chemokine (C-C motif) ligand 4-like 2 | HGNC:24066 |
| OSM | oncostatin M | HGNC:8506 |
| SAA1 | serum amyloid A1 | HGNC:10513 |
| FABP4 | fatty acid binding protein 4, adipocyte | HGNC:3559 |
| IL20 | interleukin 20 | HGNC:6002 |
| GZMB | granzyme B (granzyme 2, cytotoxic T-lymphocyte- | HGNC:4709 |
| | associated serine esterase 1) | |
| CD69 | CD69 molecule | HGNC:1694 |
| SAA2 | serum amyloid A2 | HGNC:10514 |
| LDHC | lactate dehydrogenase C | HGNC:6544 |
| FABP6 | atty acid binding protein 6, ileal | HGNC: 2172 |
| FABP7 | fatty acid binding protein 7, brain | HGNC: 2173 |
| MUC7 | mucin 7, secreted | HGNC:4589 |
| MUC1 | mucin 1, cell surface associated | HGNC: 4582 |
| SOAT1 | sterol O-acyltransferase 1 | HGNC: 6646 |
| CTNNB1 | catenin (cadherin-associated protein), beta 1, 88 kDa | HGNC: 1499 |
| GAGE2C G | antigen 2C | HGNC: 2574 |
| OR52E8 | olfactory receptor, family 52, subfamily E, mmber 8 | HGNC: 390079 |
| MAPK1 | mitogen-activated protein kinase 1 | HGNC:5594 |
| STAT3 | signal transducer and activator of transcription 3 (acute- | HGNC:6774 |
| | phase response factor) | |
| | nuclear factor of kappa light polypeptide gene enhancer in | HGNC: 4790 |
| NFKB1 | B-cells 1 | |

TABLE 6
A reduced List of Top 23 gene signatures controlling the expression of skin-
aging-related genes in genetic networks

| Symbol | Name |
|---|---|
| IL-family | interleukin |
| CSF3 | colony stimulating factor 3 (granulocyte) |
| IFNG | interferon, gamma |
| CXCL-family | chemokine (C-X-C motif) ligand |
| NR4A1 | nuclear receptor subfamily 4, group A, member 1 |
| SOCS3 | suppressor of cytokine signaling 3 |
| SAA4 | serum amyloid A4, constitutive |
| PCK1 | phosphoenolpyruvate carboxykinase 1 (soluble) |
| LIPE | lipase, hormone-sensitive |
| LEP | leptin |
| CCL4L2 | chemokine (C-C motif) ligand 4-like 2 |
| OSM | oncostatin M |
| SAA1 | serum amyloid A1 |
| FABP4 | fatty acid binding protein 4, adipocyte |
| GZMB | granzyme B (granzyme 2, cytotoxic T-lymphocyte- |
| | associated serine esterase 1) |
| CD69 | CD69 molecule |
| SAA2 | serum amyloid A2 |
| LDHC | lactate dehydrogenase C |
| FABP6 | atty acid binding protein 6, ileal |
| FABP7 | fatty acid binding protein 7, brain |
| MUC7 | mucin 7, secreted |
| MUC1 | mucin 1, cell surface associated |
| SOAT1 | sterol O-acyltransferase 1 |

1. A method for reducing or reversing the visible signs of skin ageing, the method comprising up-regulating or down-regulating expression of two or more of the following regulators: STAT1, CEBPD, MAF, RXRA, NR2B1, NR1H3, TFE3, NFATC1, RBL, SMAD3, and NR2F1.

2. (canceled)

3. (canceled)

4. The method of claim 1 further comprising recalibrating the expression of two or more pathways expressed in human skin tissue the expression of which has changed as a result of aging wherein the pathways are selected form the IL-6 pathway, cholesterol biosynthesis, and proximal lipid metabolism pathway.

5. (canceled)

6. (canceled)

7. A method for identifying a compound that will up-regulate or down-regulate the activity of one or more metabolic pathways associated with ageing, the method comprising: (i) providing a skin tissue sample, (ii) measuring the expression of a selected plurality of aging-related genes selected from the group consisting of the genes of Table 4, (iii) exposing skin tissue to a test compound, (iv) re-measuring the expression of the genes, (v) identifying a compound that changes the expression of the selected plurality of genes wherein the change in expression is at least two-fold.

8. The method of claim 7 wherein the plurality of aging-related genes comprise at least the genes encoding interleukin, colony stimulating factor 3, and interferon gamma.

9. The method of claim 7 wherein the plurality of aging-related genes comprise at least the genes encoding IL6, BARX1, CFS3, IFNG, CSAG3, PRHOXNB, IP8, LPA, CXCL2, FOXD4L6, OR1L8, IGLL5, and CXCL9.

10. The method of claim 7 wherein the plurality of aging-related genes comprise at least the genes encoding interleukin 6, colony stimulating factor 3, interferon gamma, interleukin 8, chemokine (C-X-C motif) ligand 2, chemokine (C-X-C motif) ligand 9, chemokine (C-X-C motif) ligand 10, and chemokine (C-X-C motif) ligand 1.

11. The method of claim 7 wherein the plurality of aging-related genes are shown in table 4 to be differentially expressed by a factor of at least fc (log 2) of at least 4.

12. The method of claim 8 wherein the plurality of aging-related genes are shown in table 4 to be differentially expressed by a factor of at least fc (log 2) of at least 4.

13. The method of claim 9 wherein the plurality of aging-related genes are shown in table 4 to be differentially expressed by a factor of at least fc (log 2) of at least 4.

14. The method of claim 10 wherein the plurality of aging-related genes are shown in table 4 to be differentially expressed by a factor of at least fc (log 2) of at least 4.

15. A method for identifying new compounds that alter aging-related gene expression profiles and signatures, the method comprising: (i) providing a skin tissue sample, (ii) measuring the expression of a selected plurality of aging-related genes, networks or regulators, (iii) exposing skin tissue to a test compound, (iv) re-measuring the expression of the genes, (v) identifying a compound that changes the expression of the selected plurality of genes wherein the change in expression is at least two-fold.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method of claim 15 wherein said measuring the expression of a selected plurality of aging-related genes comprises measuring the expression of a genetic regulatory module selected from the group consisting of IL-6, IL-8, IFNG, and CSF3.

35. The method of claim 43 wherein said measuring the expression of a selected plurality of aging-related genes comprises measuring the expression of a specie selected from the group consisting of: STAT1, CEBPD, MAF, RXRA (NR2B1), NR1H3 (LXR-alpha), TFE3, NFATC1, RBL2, SMAD3, and NR2F1.
